SEQUENCE LISTING (93 sequences)

SEQ ID NO: 1 - protein, 152 aa, artificial
  IL7
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 152 aa, artificial
  IL7-SS1
DSDIEGKDGKQYESVLMVSIDQLLDSMKEIGSNSLNNEFNFFKRHICDANKEGMFLFRAARKLRQFLKMNSTGDFDLHLLKVSEGTTILLNCTGQVKGRKPAALGEAQPTKSLEENKSLKEQKKLNDLSFLKRLLQEIKTSWNKILMGTKEH

SEQ ID NO: 3 - protein, 152 aa, artificial
  IL7-SS2
DSDIEGKDGKQYESVLMVSIDQLLDSMKEIGSNCLNNEFNFFKRHISDANKEGMFLFRAARKLRQFLKMNSTGDFDLHLLKVSEGTTILLNSTGQVKGRKPAALGEAQPTKSLEENKSLKEQKKLNDLCFLKRLLQEIKTSWNKILMGTKEH

SEQ ID NO: 4 - protein, 152 aa, artificial
  IL7-SS3
DCDIEGKDGKQYESVLMVSIDQLLDSMKEIGSNSLNNEFNFFKRHISDANKEGMFLFRAARKLRQFLKMNSTGDFDLHLLKVSEGTTILLNSTGQVKGRKPAALGEAQPTKSLEENKSLKEQKKLNDLSFLKRLLQEIKTCWNKILMGTKEH

SEQ ID NO: 5 - protein, 152 aa, artificial
  IL7-W142H
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 6 - protein, 152 aa, artificial
  IL7-W142Y
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCYNKILMGTKEH

SEQ ID NO: 7 - protein, 152 aa, artificial
  IL7-W142F
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCFNKILMGTKEH

SEQ ID NO: 8 - protein, 152 aa, artificial
  IL7-Q11E
DCDIEGKDGKEYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 9 - protein, 152 aa, artificial
  IL7-Y12F
DCDIEGKDGKQFESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 10 - protein, 152 aa, artificial
  IL7-M17L
DCDIEGKDGKQYESVLLVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 11 - protein, 152 aa, artificial
  IL7-Q22E
DCDIEGKDGKQYESVLMVSIDELLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 12 - protein, 152 aa, artificial
  IL7-D74E
DCDIEGKDGKQYESVLMVSIDQLLDSMKEIGSNCLNNEFNFFKRHICDANKEGMFLFRAARKLRQFLKMNSTGEFDLHLLKVSEGTTILLNCTGQVKGRKPAALGEAQPTKSLEENKSLKEQKKLNDLCFLKRLLQEIKTCWNKILMGTKEH

SEQ ID NO: 13 - protein, 152 aa, Artificial Sequence
  IL7-D74Q
DCDIEGKDGKQYESVLMVSIDQLLDSMKEIGSNCLNNEFNFFKRHICDANKEGMFLFRAARKLRQFLKMNSTGQFDLHLLKVSEGTTILLNCTGQVKGRKPAALGEAQPTKSLEENKSLKEQKKLNDLCFLKRLLQEIKTCWNKILMGTKEH

SEQ ID NO: 14 - protein, 152 aa, artificial
  IL7-D74N
DCDIEGKDGKQYESVLMVSIDQLLDSMKEIGSNCLNNEFNFFKRHICDANKEGMFLFRAARKLRQFLKMNSTGNFDLHLLKVSEGTTILLNCTGQVKGRKPAALGEAQPTKSLEENKSLKEQKKLNDLCFLKRLLQEIKTCWNKILMGTKEH

SEQ ID NO: 15 - protein, 152 aa, artificial
  IL7-K81R
DCDIEGKDGKQYESVLMVSIDQLLDSMKEIGSNCLNNEFNFFKRHICDANKEGMFLFRAARKLRQFLKMNSTGDFDLHLLRVSEGTTILLNCTGQVKGRKPAALGEAQPTKSLEENKSLKEQKKLNDLCFLKRLLQEIKTCWNKILMGTKEH

SEQ ID NO: 16 - protein, 9 aa, artificial
  LC CDR3
FQGTHVPNT

SEQ ID NO: 17 - protein, 117 aa, artificial
  HC variable domain
  MISC_FEATURE (105)..(105): X is X1, D or E
  MISC_FEATURE (106)..(106): X is X2, and is T, H, A, Y, N, E or S
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMXXWGQGTLVTVSS

SEQ ID NO: 18 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDSWGQGTLVTVSS

SEQ ID NO: 19 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDTWGQGTLVTVSS

SEQ ID NO: 20 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMESWGQGTLVTVSS

SEQ ID NO: 21 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDHWGQGTLVTVSS

SEQ ID NO: 22 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDAWGQGTLVTVSS

SEQ ID NO: 23 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDYWGQGTLVTVSS

SEQ ID NO: 24 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSS

SEQ ID NO: 25 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDEWGQGTLVTVSS

SEQ ID NO: 26 - protein, 112 aa, artificial
  LC variable domain
  MISC_FEATURE (34)..(34): X is G or T
DVVMTQSPLSLPVTLGQPASISCRSSQSLVHANXNTYLEWYQQRPGQSPRLLIYKVSNRFSGVPDRFSGSGSGTDFTLKISRVEAEDVGVYYCFQGTHVPNTFGQGTKLEIK

SEQ ID NO: 27 - protein, 112 aa, artificial
  LC variable domain
DVVMTQSPLSLPVTLGQPASISCRSSQSLVHANGNTYLEWYQQRPGQSPRLLIYKVSNRFSGVPDRFSGSGSGTDFTLKISRVEAEDVGVYYCFQGTHVPNTFGQGTKLEIK

SEQ ID NO: 28 - protein, 112 aa, artificial
  LC variable domain
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIK

SEQ ID NO: 29 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 30 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 31 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMESWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 32 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 33 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 34 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 35 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 36 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 37 - protein, 219 aa, artificial
  LC
DVVMTQSPLSLPVTLGQPASISCRSSQSLVHANGNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 219 aa, artificial
  LC
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 327 aa, artificial
  HC constant domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 40 - protein, 107 aa, artificial [sequence omitted]

SEQ ID NO: 41 - protein, 30 aa, artificial
  HFR1
QIQLVQSGSELKKPGASVKVSCKASGYTFT

SEQ ID NO: 42 - protein, 14 aa, artificial
  HFR2
WVRQAPGQGLEWMG

SEQ ID NO: 43 - protein, 32 aa, artificial
  HFR3
RFVFSLDTSVSTAYLQISSLKAEDTAVYYCAR

SEQ ID NO: 44 - protein, 11 aa, artificial
  HFR4
WGQGTLVTVSS

SEQ ID NO: 45 - protein, 23 aa, artificial
  LFR1
DVVMTQSPLSLPVTLGQPASISC

SEQ ID NO: 46 - protein, 15 aa, artificial
  LFR2
WYQQRPGQSPRLLIY

SEQ ID NO: 47 - protein, 32 aa, artificial
  LFR3
GVPDRFSGSGSGTDFTLKISRVEAEDVGVYYC

SEQ ID NO: 48 - protein, 10 aa, artificial
  LFR4
FGQGTKLEIK

SEQ ID NO: 49 - protein, 19 aa, artificial
  signal peptide
MDWLWNLLFLMAAAQSIQA

SEQ ID NO: 50 - protein, 19 aa, artificial
  signal peptide
MKLPVRLLVLMFWIPASSS

SEQ ID NO: 51 - protein, 5 aa, artificial
  HC CDR1
HYAMN

SEQ ID NO: 52 - protein, 330 aa, artificial
  Heavy chain constant domain (IgG1m-N298A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 17 aa, artificial
  HC CDR2
WINTNTGEPTYAQGFTG

SEQ ID NO: 54 - protein, 8 aa, artificial
  HC CDR3
  MISC_FEATURE (7)..(7): X is X1 (D or E)
  MISC_FEATURE (8)..(8): X is X2 and is T, H, A, Y, N, E or S
EREPGMXX

SEQ ID NO: 55 - protein, 8 aa, artificial
  HC CDR3
EREPGMDS

SEQ ID NO: 56 - protein, 8 aa, artificial
  HC CDR3
EREPGMDT

SEQ ID NO: 57 - protein, 8 aa, artificial
  HC CDR3
EREPGMES

SEQ ID NO: 58 - protein, 8 aa, artificial
  HC CDR3
EREPGMDH

SEQ ID NO: 59 - protein, 8 aa, artificial
  HC CDR3
EREPGMDA

SEQ ID NO: 60 - protein, 8 aa, artificial
  HC CDR3
EREPGMDY

SEQ ID NO: 61 - protein, 8 aa, artificial
  HC CDR3
EREPGMDN

SEQ ID NO: 62 - protein, 8 aa, artificial
  HC CDR3
EREPGMDE

SEQ ID NO: 63 - protein, 16 aa, artificial
  LC CDR1
  MISC_FEATURE (11)..(11): X is G or T
RSSQSLVHANXNTYLE

SEQ ID NO: 64 - protein, 16 aa, artificial
  LC CDR1
RSSQSLVHANGNTYLE

SEQ ID NO: 65 - protein, 16 aa, artificial
  LC CDR1
RSSQSLVHANTNTYLE

SEQ ID NO: 66 - protein, 7 aa, artificial
  LC CDR2
KVSNRFS

SEQ ID NO: 67 - protein, 9 aa, artificial
  linker C
GGGGSGGGS

SEQ ID NO: 68 - protein, 5 aa, artificial
  linker G4S
GGGGS

SEQ ID NO: 69 - protein, 10 aa, artificial
  linker (G4S)2
GGGGSGGGGS

SEQ ID NO: 70 - protein, 15 aa, artificial
  linker (G4S)3
GGGGSGGGGSGGGGS

SEQ ID NO: 71 - protein, 444 aa, artificial
  HC anti-PD1 chimeric
QIQLVQSGPELKKPGETVKISCKASGYTFTHYGMNWVKQAPGKGLKWMGWINTNTGEPTYAEEFKGRFAFSLETSASAAFLQINNLKNEDTATYFCAKEREPGMDSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 72 - protein, 219 aa, artificial
  LC anti-PD1 chimeric
DVLLTQTPLSLPVSLGDQASISCRSSQNIVHANGNTYLEWYLQKPGQSPKLLIYKVSNRFSGVPDRFSGSGSGTDFTLKITRVEAEDLGVYYCFQGSHVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 2670 bp, artificial
  VH-VK
cagatccagctggtgcagagcggctctgagctgaagaagccaggcgcttctgtgaaggtgtcctgcaaggccagcggctacaccttcacacactatgctatgaattgggtgagacaggctccaggacagggactggagtggatgggctggatcaacaccaatacaggcgagcctacctacgctcagggctttacaggccgcttcgtgttttctctggatacctccgtgagcacagcctatctgcagatctccagcctgaaggctgaggacaccgccgtgtactattgtgctagggagagggagccaggaatggataactggggacagggcaccctggtgacagtgtcttccgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatgacagatccagctggtgcagagcggctctgagctgaagaagccaggcgcttctgtgaaggtgtcctgcaaggccagcggctacaccttcacacactatgctatgaattgggtgagacaggctccaggacagggactggagtggatgggctggatcaacaccaatacaggcgagcctacctacgctcagggctttacaggccgcttcgtgttttctctggatacctccgtgagcacagcctatctgcagatctccagcctgaaggctgaggacaccgccgtgtactattgtgctagggagagggagccaggaatggataactggggacagggcaccctggtgacagtgtcttccgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 74 - DNA, 660 bp, artificial
  VL-VD
gacgtggtcatgacacagagcccactgtctctgcctgtgaccctgggacagccagcctctatctcctgcagatccagccagtctctggtgcacgctaacaccaatacatacctggagtggtatcagcagaggccaggacagtccccaaggctgctgatctacaaggtgtccaacagattcagcggagtgccagaccgctttagcggatctggatccggaaccgacttcaccctgaagatctccagggtggaggctgaggatgtgggcgtgtactattgtttccagggcacccatgtgcctaatacatttggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 75 - protein, 227 aa, artificial
  KIH-FcG1m (Knob)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 394 aa, artificial
  KIH-FcG1m-(G4S)3-W142H (Knob)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 77 - protein, 227 aa, artificial
  KIH-FcG1m (Hole)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 78 - protein, 394 aa, artificial
  KIH-FcG1m-(G4S)3-W142H (Hole)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 79 - protein, 220 aa, artificial
  Anti-PD-1-CH1
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 80 - protein, 219 aa, artificial; identical to SEQ ID NO: 38
  Anti-PD-1-CL

SEQ ID NO: 81 - protein, 447 aa, artificial
  Anti-PD-1-CH1-KIH-FcG1m (Knob)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 614 aa, artificial
  Anti-PD-1-CH1-KIH-FcG1m-(G4S)3-W142H (Knob)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 83 - protein, 614 aa, artificial
  Anti-PD-1-CH1-KIH-FcG1m-(G4S)3-W142H (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 84 - protein, 595 aa, artificial
  Anti-PD-1-CH1-KIH-FcG1m-(G4S)3-IL2 (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 85 - protein, 576 aa, artificial
  anti-PD-1-CH1-KIH-FcG1m-(G4S)3-IL15 (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 86 - protein, 600 aa, artificial
  anti-PD-1-CH1-KIH-FcG1m-(G4S)3-IL21 (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 87 - protein, 133 aa, Homo sapiens [sequence omitted]

SEQ ID NO: 88 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 89 - protein, 138 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 90 - protein, 447 aa, artificial
  anti-PD-1-CH1-KIH-FcG1m (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 91 - protein, 614 aa, artificial
  anti-PD-1+CH1  IL7wt
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 92 - protein, 614 aa, artificial
  anti-PD-1+CH1  IL7 W142H
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 93 - protein, 614 aa, artificial
  anti-PD-1-CH1-KIH-FcG1mIL7WT (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

(19) United States
(12) Patent Application Publication
(10) Pub. No.: US 2024/0182571 A1
(43) Pub. Date: Jun. 6, 2024

(54) NEW SCAFFOLD FOR BIFUNCTIONAL MOLECULES WITH IMPROVED PROPERTIES

(71) Applicant: OSE IMMUNOTHERAPEUTICS, NANTES (FR)

(72) Inventors: NICOLAS POIRIER, NANTES (FR); AURORE MORELLO, NANTES (FR); CAROLINE MARY, NANTES (FR); MARGAUX SEITÉ, NANTES (FR)

(21) Appl. No.: 18/285,659

(22) PCT Filed: Apr. 8, 2022
(86) PCT No.: PCT/EP2022/059414

§ 371 Date: Oct. 5, 2023

(30) Foreign Application Priority Data
Apr. 9, 2021 (EP) 21305462.0
Sep. 30, 2021 (EP) 21200350.3

(51) Int. Cl. C07K 16/28; A61K 38/00; A61K 39/00; A61P 35/00; C07K 14/54; C07K 14/55

(52) U.S. Cl. CPC C07K 16/2818; A61P 35/00; C07K 14/54; C07K 14/5418; C07K 14/5443; C07K 14/55; C07K 16/28; C07K 16/2803; A61K 38/00; A61K 39/00; C07K 2317/569; C07K 2317/622; C07K 2319/30

(57) ABSTRACT

The present invention relates to bifunctional molecules having a particular scaffold and their uses.

### FIELD OF THE INVENTION

The invention pertains to the field of immunotherapy. The present invention provides a new scaffold for bifunctional molecules and their uses in medicine.

### BACKGROUND OF THE INVENTION

Bifunctional molecules are currently the object of developments in immunology, especially in the field of oncology. Indeed, they bring novel pharmacological properties through the co-engagement of two targets, may increase the safety profile as compared to a combination of two distinct molecule thanks to a targeted relocation to the tumor and may potentially reduce development and manufacturing costs associated with single drug product. However, these molecules are advantageous but may also present several inconveniences. The design of bifunctional molecules need to imply several key attributes such as binding affinity and specificity, folding stability, solubility, pharmacokinetics, effector functions, compatibility with the attachment of additional domains and production yield and cost compatible with a clinical developments.

Bifunctional molecules based on an antibody antagonizing PD-1 and linked to IL-7, or other immunotherapeutic agents have been disclosed, in particular in WO 2020/127377 and WO 2020/127366. However, there is still a strong need of improved scaffold for bifunctional molecules.

### SUMMARY OF THE INVENTION

The present invention relates to a bifunctional molecule having a particular scaffold and comprising a single monovalent antigen binding domain that binds a target specifically expressed on immune cells surface and a single immuno-stimulating cytokine. This scaffold is essentially made of a dimeric Fc domain, a single monovalent antigen binding domain that binds a target specifically expressed on immune cells surface linked at the N terminal end of one monomer of the Fc domain and either i) a single immuno-stimulating cytokine linked at the C terminal end of the same monomer of the Fc domain or ii) the single monovalent antigen binding domain comprises a heavy variable chain and a light variable chain and the single immuno-stimulating cytokine is linked at the C terminal end of the light chain of antigen binding domain.

This particular scaffold is associated with an improved pharmacokinetic profile. This improvement has been observed with bifunctional molecules comprising different immune-stimulating moieties, such as IL-2; IL-7, IL-15 and IL-21. The improved pharmacokinetic profile is surprising because, in absence of the immuno-stimulating cytokine, the improvement is not observed for this scaffold. The bifunctional molecule with this particular scaffold is favorable to cis-targeting of the two targets on the same cells, allowing a selective delivery of the immune-stimulating cytokine to the targeted cells. In addition, in the context of a bifunctional molecule having IL-7, these molecules are able to induce a synergistic activation and a better in vivo anti-tumor efficacy. Finally, surprisingly, the bifunctional molecules having a particular scaffold have a better productivity and avoid the side products due to chain mispairing which is a major advantage for production at an industrial scale and safety. Besides, in addition to the improved pharmacokinetic profile and the better productivity, a new and advantageous biological efficiency has been identified for some compounds (notably those having IL7) which improve the activity of effector memory stem like T cell, a subset of tumor-reactive intra-tumoral T cells of strong interest in view of their immune activity.

Accordingly, the present invention relates to a bifunctional molecule comprising a single antigen binding domain that binds to a target specifically expressed on immune cells surface and a single immuno-stimulating cytokine,
wherein the molecule comprises a first monomer comprising an antigen-binding domain covalently linked via C-terminal end to N-terminal end of a first Fc chain, optionally via a peptide linker, and a second monomer comprising a complementary second Fc chain devoid of antigen-binding domain and of the immuno-stimulating cytokine;
wherein either i) the immuno-stimulating cytokine is covalently linked to the C-terminal end of said first Fc chain, optionally via a peptide linker; or ii) the single antigen binding domain comprises a heavy variable chain and a light variable chain and the immuno-stimulating cytokine is covalently linked to the C-terminal end of the light chain;
wherein the target specifically expressed on immune cells surface is selected from the group consisting of PD-1, CD28, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CD3, PDL2, and PDL1; and
wherein the immuno-stimulating cytokine is selected from the group consisting of IL-2 (IL being interleukin), IL-4, IL-5, IL-6, IL-12A, IL-12B, IL-13; IL-15, IL-18, IL-21, IL-23, IL-24; IFNα, IFNβ, BAFF, LTα, and LTβ, or a variant thereof having at least 80% of identity with the wildtype protein or the extracellular fragment thereof and IL-7.

In a particular aspect, the immuno-stimulating cytokine is linked at the C-terminal end of first Fc chain, preferably by its N-terminal end.

In a particular aspect, the first Fc chain and the second Fc chain form a heterodimeric Fc domain, in particular a knob-into-hole heterodimeric Fc domain.

Optionally, the immuno-stimulating cytokine is selected from the group consisting of IL-2, IL-15, and IL-21, or a variant thereof having at least 80% of identity with the wildtype protein, and IL-7.

In a particular aspect, the immuno-stimulating cytokine is IL-7, for example such as described under the sequence set forth in SEQ ID NO: 1.

In another particular aspect, the immuno-stimulating cytokine is IL-2 or a variant thereof having at least 90% of identity with SEQ ID NO: 87, preferably selected from an IL-2 variant comprising one of the following substitutions combination relative to SEQ ID NO: 87: R38E and F42A; R38D and F42A; F42A and E62Q; R38A and F42K; R38E, F42A, and N88S; R38E, F42A, and N88A; R38E, F42A, and V91E; R38E, F42A, and D84H; H16D, R38E and F42A; H16E, R38E and F42A; R38E, F42A and Q126S; R38D, F42A and N88S; R38D, F42A and N88A; R38D, F42A and V91E; R38D, F42A, and D84H; H16D, R38D and F42A; H16E, R38D and F42A; R38D, F42A and Q126S; R38A, F42K, and N88S; R38A, F42K, and N88A; R38A, F42K, and V91E; R38A, F42K, and D84H; H16D, R38A, and F42K; H16E, R38A, and F42K; R38A, F42K, and Q126S; F42A, E62Q, and N88S; F42A, E62Q, and N88A; F42A, E62Q, and V91E; F42A, E62Q, and D84H; H16D, F42A, and E62Q; H16E, F42A, and E62Q; F42A, E62Q, and Q126S; R38E, F42A, and C125A; R38D, F42A , and C125A; F42A, E62Q, and C125A; R38A, F42K, and C125A; R38E, F42A, N88S, and C125A; R38E, F42A, N88A, and C125A; R38E, F42A, V91E, and C125A; R38E, F42A, D84H, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, V91E, and C125A; R38D, F42A, D84H, and C125A; H16D, R38D, F42A, and C125A; H16E, R38D, F42A, and C125A; R38D, F42A , C125A, and Q126S; R38A, F42K, N88S, and C125A; R38A, F42K, N88A, and C125A; R38A, F42K, V91E, and C125A; R38A, F42K, D84H, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, V91E, and C125A; F42A, E62Q, and D84H, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, V91E, and C125A; F42A, D84H, and C125A; H16D, F42A, and C125A; H16E, F42A, and C125A; F42A, C125A and Q126S; F42A, Y45A and L72G; T3A, F42A, Y45A, L72G and C125A; or at least one of the substitutions selected in the group comprising K35E, K35A, R38A, R38E, R38N, R38F, R38S, R38L, R38G, R38Y, R38W, F42L, F42A, F42G, F42S, F42T, F42Q, F42E, F42N, F42D, F42R, F42K, K43E, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K; or a combination thereof, preferably the three substitutions F42A, Y45A and L72G.

In another particular aspect, the immuno-stimulating cytokine is IL-15 or a variant thereof having at least 90% of identity with SEQ ID NO: 88, preferably selected from an IL-15 variant comprising one of the following substitutions relative to SEQ ID NO: 88: N1D, V3I, V3M, V3R, N4D, D8N, D8A, K11L, K11M, K11R, D30N, D61N, E64Q, N65D, N71D, N71S, N72D, N72A, N72R, N72Y, S73I, N77A, N79D, N79E, N79S, Q108E, N112D, N112H, N112M and N112Y, preferably N4D, D61N, N65D, Q108E, N4D/N65D, D30N/N65D, D30N/E64Q, D30N/E64Q/N65D, N1D, N4D, D8N, D30N, D61N, E64Q, N65D, Q108E, N1D/D61N, N1D/E64Q, N4D, D61N, N4D/E64Q, D8N/D61N, D8N/E64Q, D61N/E64Q, N1D/D30N, E64Q/Q108E, N1D/N4D/D8N, D61N/E64Q/N65Q, N1D/D61N/E64Q/Q108E, N4D/D61N, N4D/D61N/E64Q/Q108E, N1D/N65D, N1D/Q108E, N4D/D30N, D30N/Q108E, N65D/Q108E, D30N/Q180E, E64Q/N65D, D61N/E64Q/N65D, N1D/N4D/N65D, N71S/N72A/N77A, N4D/D61N/N65D, preferably D30N/E64Q/N65D.

In another particular aspect, the immuno-stimulating cytokine is IL-21 or a variant thereof having at least 90% of identity with SEQ ID NO: 89, preferably selected from an IL-21 variant comprising one of the following substitutions relative to SEQ ID NO: 89: R5A, R5D, R5E, R5G, R5H, R5I, R5K, R5L, R5M, R5N, R5Q, R5S, R5T, R5V, R5Y, 18A, 18D, 18E, 18G, 18N, 18S, R9A, R9D, R9E, R9G, R9H, R9I, R9K, R9L, R9M, R9N, R9Q, R9S, R9T, R9V, R9Y, R11D, R11S, Q12A, Q12D, Q12E, Q12N, Q12S, Q12T, Q12V, L13D, I14A, I14D, I14S, D15A, D15E, D15I, D15M, D15N, D15Q, D15S, D15T, D15V, I16D, I16E, Q19D, Y23D, R65D, R65G, R65P, I66D, I66G, I66P, N68Q, V69D, V69G, V69P, S70E, S70G, S70P, S70Y, S70T, K72D, K72G, K72P, K72A, K73A, K73D, K73E, K73G, K73H, K73I, K73N, K73P, K73Q, K73S, K73V, K75D, K75G, K75P, R76A, R76D, R76E, R76G, R76H, R76I, R76K, R76L, R76M, R76N, R76P, R76Q, R76S, R76T, R76V, R76Y, K77D, K77G, K77P, P78D, P79D, S80G, S80P, E109K, R110D, K112D, S113K, Q116A, Q116D, Q116E, Q116I, Q116K, Q116L, Q116M, Q116N, Q116S, Q116T, Q116V, K117D, I119A , I119D, I119E, I119M, I119N, I119Q, I119S, I119T, H120D and L123D, preferably R5E and R76E, R5E and R76A, R5A and R76A, R5Q and R76A, R5A and R76E, R5Q and R76E, R9E and R76E, R9A and R76E, R9E and R76A, R9A and R76A, D15N and S70T, D15N and I71L, D15N and K72A, D15N and K73A, S70T and K73Q, S70T and R76A, S70T and R76D, S70T and R76E, I71L and K73Q, I71L and R76A, I71L and R76D, I71L and R76E, K72A and K73Q, K72A and R76A, K72A and R76D, K72A and R76E, K73A and R76A, K73A and R76D, or K73A and R76E.

Optionally, the antigen-binding domain is a Fab domain, a Fab′, a single-chain variable fragment (scFV) or a single domain antibody (sdAb).

In a particular aspect, the target specifically expressed on immune cells surface is selected from the group consisting of PD-1, CTLA-4, BTLA, TIGIT, LAG3 and TIM3, preferably PD-1.

In a very particular aspect, the antigen binding domain comprises or consists essentially of: (i) a heavy chain comprising a CDR1 of SEQ ID NO: 51, a CDR2 of SEQ ID NO: 53 and a CDR3 of SEQ ID NO: 55, 56, 57, 58, 59, 60, 61 or 62; and (ii) a light chain comprising a CDR1 of SEQ ID NO: 64 or 65, a CDR2 of SEQ ID NO: 66 and a CDR3 of SEQ ID NO: 16.

The present invention also relates to an isolated nucleic acid sequence or a group of isolated nucleic acid molecules encoding the bifunctional molecule according to the present disclosure, and a host cell comprising the isolated nucleic acid(s).

The present invention further relates to a pharmaceutical composition comprising the bifunctional molecule, the nucleic acid(s) or the host cell according to the present disclosure, optionally with a pharmaceutically acceptable carrier.

Finally, the present invention relates to the bifunctional molecule, the nucleic acid(s), the host cell or the pharmaceutical composition according to the present disclosure for use as a medicament, especially for use in the treatment of a cancer or an infectious disease; the use of the bifunctional molecule, the nucleic acid(s), the host cell or the pharmaceutical composition according to the present disclosure for the manufacture of a medicament, especially for use in the treatment of a cancer or an infectious disease; and to a method of treating of a disease, especially a cancer or an infectious, in a subject comprising administering a therapeutically effective amount of the bifunctional molecule, the nucleic acid(s), the host cell or the pharmaceutical composition according to the present disclosure.

Optionally, the present invention relates to the bifunctional molecule, the nucleic acid(s), the host cell or the pharmaceutical composition according to the present disclosure for use in the treatment of a cancer or a viral infection by stimulating of effector memory stem like T cells; to the use of the bifunctional molecule, the nucleic acid(s), the host cell or the pharmaceutical composition according to the present disclosure for the manufacture of a medicament, especially for use in the treatment of a cancer or a viral infection by stimulating of effector memory stem like T cells; to a method of treating of a cancer or a viral infection, in a subject comprising administering a therapeutically effective amount of the bifunctional molecule, the nucleic acid(s), the host cell or the pharmaceutical composition according to the present disclosure, thereby stimulating effector memory stem like T cells.

### BRIEF DESCRIPTION OF THE DRAWINGS

FIG. 1: Schematic representation of the different molecules used in the examples 1 and 2. Below the schematic representation of construction 3 is another representation of such construction, in which each chain and components of the molecule is further described.

FIG. 2: Anti PD-1 IL7 W142H mutant demonstrates high binding efficiency to PD-1 and antagonizes PDL1 binding. A. PD-1 binding ELISA assay. Human recombinant PD-1 (rPD1) protein was immobilized, and antibodies were added at different concentrations. Revelation was performed with an anti-human Fc antibody coupled to peroxidase. Colorimetry was determined at 450 nm using TMB substrate. The anti PD-1 with 1 (anti PD-1*1 [symbol] grey) or 2 anti PD-1 arms (anti PD-1*2 ♦) were tested as controls. The bifunctional molecules comprising an IL7 variant (anti PD-1*2 IL7 W142H*2 ● black), (anti PD-1*2 IL7 W142H*1 ▪ black), (anti PD-1*1 IL7 W142H*2 [symbol] grey), (anti PD-1*1 IL7 W142H*1 [symbol] grey) were also tested. B. Antagonistic capacity to block PD-1/PD-L1 measured by ELISA. PD-L1 was immobilized, and the complex antibodies+biotinylated recombinant human PD-1 was added. This complex was generated with a fixed concentration of PD1 (0.6 μg/mL) and different concentrations of anti-PD1*2 IL7 W142H*1 (▪ plain line), anti-PD1*2 IL7 W142H*2(○ dashed line), anti PD-1*1 (grey [symbol] dashed grey line), anti-PD1*1 IL7 W142H*2 (grey [symbol] plain grey line) or anti-PD1*1 IL7 W142H*1 (grey [symbol] plain grey line). All constructions tested comprise a GGGGSGGGGSGGGGS linker between the Fc and IL-7 domain.

FIG. 3: Anti PD-1 IL7 molecules constructed with one valence of anti PD-1 and one IL-7 W142H cytokine activate pSTAT5 with high efficacy. pSTAT5 signaling assay after treatment with anti PD-1*1 IL7 W142H*1 (●) anti PD-1*2 IL7WT*2 (▪) or anti-PD1*2 IL7 W142H*1 (▴). All W142H constructions tested comprise an IgG1m and a GGGGSGGGGSGGGGS linker between the Fc and IL-7 domain.

FIG. 4: Anti PD-1*2 IL7*1, Anti PD-1*1 IL7*1, Anti PD-1*1 IL7*2 W142H mutants preferentially bind and activate pSTAT5 signaling into PD-1+ CD127+ cells over PD-1-CD127+ cells. U937 cells expressing CD127+ or co-expressing CD127+ and PD-1+ cells were stained with a cell proliferation dye (CPDe450 or CPDe670) and co-cultivated at ratio 1:1 prior incubation with different concentrations of anti PD-1 IL-7 bifunctional molecules. Staining with and anti-human IgG PE and pSTAT5 activation was quantified after incubation by flow cytometry. A. EC50 binding (nM) was calculated for each cell type and each construction. B. EC50 pSTAT5 (nM) was calculated for each cell type and each construction. After treatment with bifunctional molecules, cells were then fixed, permeabilized and stained with an AF647 labeled anti-pSTAT5 (clone 47/Stat5(pY694). pSTAT5 activation. EC50 (nM) was calculated for each construction and each cell type U937 PD-1+ CD127+ (white histogram) and U937 PD-1− CD127+ (black histogram). n=2 independent experiments. In this assay, anti PD-1*2 IL7 W142*1, anti PD-1*1 IL7 W142*1 and anti PD-1*1 IL7 W142*2 were tested and comprise an IgG1m isotype and a GGGGSGGGGGGGGS linker between the Fc and IL-7 domain.

FIG. 5: Pharmacokinetics of the Anti PD-1*2 IL7*1, Anti PD-1*1 IL7*1, Anti PD-1*1 IL7*2 W142H mutant molecules following intraperitoneal injection. humanized PD1 mice were intraperitoneally injected with one dose (34 nM/kg) of the anti PD-1*2 IL7 IL7*2 IgG4m (Δ), anti PD-1*2 IL7 W142H*1 IgG1m (▾), anti PD-1*1 IL7 W142H*1 IgG1m (● grey), or anti PD-1*1 IL7 W142H*2 IgG1m (○ grey). Concentration of the drugs in the sera was assessed by ELISA following injection until 72 h.

FIG. 6: Schematic structure of the anti PD-1/protX bifunctional antibodies used in the examples 3 to 9. Format A: anti PD-1*2/ProtX*2 comprises 2 anti PD-1 arms and 2 protein X fused to the C-terminal of the Fc domain of the anti PD-1 antibody. Format B: anti PD-1*2/ProtX*1 comprises 2 anti PD-1 arms and 1 protein X fused to the C-terminal of the Fc domain of the anti PD-1 antibody (Chain B). Format C: Anti PD-1*1/protX*1 comprises 1 anti PD-1 arm and 1 protein X fused to the C-terminal of the Fc domain of the anti PD-1 antibody (Chain B). Below the schematic representation of Format C is another representation of such construction, in which each chain and components of the molecule is further described. The Format B and C also comprises a Knob into Hole strategy with the Knob mutation preferably introduced into the Chain A and the Hole mutation preferably introduced into the Chain B.

FIG. 7: Productivity of anti PD-1/ProtX bifunctional antibodies in mammalian cells. CHO-S cells were transiently transfected with the DNA encoding for the anti PD-1*2/protX*1 or the anti PD-1*1/protX*1 molecules at a ratio (1:3:3; Chain A:Chain B:VL). Supernatant containing the antibodies were purified using Protein A chromatography. The quantity of bifunctional antibody obtained after purification was quantified by UV spectrometry (DO 280 nm) and normalized to the volume of production. FIG. 7A. Raw data of productivity, each dot represents the productivity of one anti PD-1*1/protX*1 antibody. FIG. 7B. Normalized productivity of all anti PD-1*1/protX*1 antibody versus anti PD-1*2/protX*1 molecules. FIG. 7C. Raw data productivity of the individual construction.

FIG. 8: Size exclusion chromatography of the anti PD-1*1/IL-7wt*1 (A) and the anti PD-1*1/IL-7v*1 (B). Purified antibodies were separated by their size using gel filtration chromatography using the SuperDex 200 (10/300GL). Peak corresponding to aggregates, heterodimer antibody and Fc homodimer are represented on the graphic with the % of compound calculated.

FIG. 9: Anti PD-1*1/protX*1 bifunctional antibodies demonstrate high binding efficiency to human PD-1. Human recombinant PD-1 (rPD1) protein was immobilized, and antibodies were added at different concentrations. Revelation was performed with an anti-human Fc antibody coupled to peroxidase. Colorimetry was determined at 450 nm using TMB substrate. FIG. 9A. Anti PD-1*1/IL-2*1, FIG. 9B. Anti PD-1*1 IL-21*1, FIG. 9C. Anti PD-1*1/IL-15*1.

FIG. 10: Anti PD-1*/cytokine*1 molecules activates pSTAT5 with high efficacy. FIG. 10A. pSTAT5 signaling assay of human primary T cells treated with the anti PD-1*1 /IL-21*1, the anti PD-1*1 /IL-15*1, the anti PD-1*1 /IL-7wt*1, the anti PD-1*1 /IL-7v*1. Human PBMCs isolated from peripheral blood of healthy volunteers were incubated 15 minutes with the molecules. Cells were then fixed, permeabilized and stained with an anti CD3-BV421 and an anti-pSTAT5 AF647 (clone 47/Stat5(pY694)). Data correspond to the % pSTAT5+ cells into CD3+ population. FIG. 10B. pSTAT5 signaling into human CD127+ CD132+ U937 cell lines after treatment with anti PD-1*2 IL7v*2 (▾) or the anti PD-1*1 IL7v1*1 (Δ) molecules. Left graph corresponds to the % of pSTAT5+ cells and the right graph corresponds to the EC50 (nM) calculated referring to the concentration required to reach 50% of the pSTAT5 activation. Data represent mean +/−SD of 3 independent experiments.

FIG. 11: Anti PD-1*1/protX*1 molecules preferentially bind with high efficiency to PD-1+ cells over PD-1− cells. FIG. 11A. U937 cells expressing PD-1+ cells and U937 PD-1− cells were stained with a cell proliferation dye (CPDe450 or CPDe670) and co-cultivated at ratio 1:1 prior incubation with different concentrations of anti PD-1*1/IL2*1, anti PD-1*1/IL15*1, Anti PD-1*1 IL-21*1 bifunctional molecules or the anti PD-1*1 et anti PD-1*2 as positive control staining. Staining with and anti-human IgG PE and pSTAT5 activation was quantified after incubation by flow cytometry in the PD-1+ (plain line) or PD-1− cells (dashed line). FIG. 11B. Binding of the Anti PD-1*1/IL-7wt*1 molecule on PD-1+ CD127+ U937 cells (plain line) versus PD-1− CD127+ U937 cell lines (dashed lines). FIG. 11C. Binding of the Anti PD-1*1/IL-7v*1 molecule on PD-1+ CD127+ U937 cells (plain line) versus PD-1− CD127+ U937 cell lines (dashed lines). FIG. 11D. Comparison of the pSTAT5 activation into PD-1+ CD127+ cells versus PD-1− cells CD127+ cells. EC50 nM ratio of pSTAT5 activation on PD-1+ CD127+(white histogram) versus PD-1− CD127+ cells (black histogram) was calculated and reported on the graphics. N=3 independent experiments.

FIG. 12: Anti PD-1*1 IL7*1 synergistically activates TCR signaling. Promega PD-1/PD-L1 bioassay: (1) Effector T cells (Jurkat stably expressing PD-1, NFAT-induced luciferase) and (2) activating target cells (CHO K1 cells stably expressing PD-L1 and surface protein designed to activate cognate TCRs in an antigen-independent manner) were co-cultured. After adding BioGlo™ luciferin, luminescence is quantified and reflects T cell activation. FIG. 12A. anti-PD1*1 (▪ black), anti PD-1*1+ Isotype*1 IL-7wt*1 as separate compound (∇), anti PD-1*1 IL7wt*1 (▴ black) were added at serial concentrations. Right graph EC50 (nM) calculated for each construction. FIG. 12B. anti-PD1*1 (▪ black), anti PD-1*1+ Isotype*1 IL-7v*1 as separate compound (∇), anti PD-1*1 IL7v*1 (▴ black) were added at serial concentrations. Right graph EC50 (nM) calculated for each construction. Data represent at least 3 independent experiments.

FIG. 13: Anti PD-1*1/IL7v*1 demonstrated higher pharmacokinetics in vivo than anti PD1*2/IL7v*1 or the anti PD-1*2/IL7v*2 molecules. C57BL/6 mice were intravenously (FIG. 13A) or intraperitoneally (FIG. 13B) injected with one dose (34 nmol/kg) of bifunctional anti PD-1/IL-7v molecules. Antibodies concentration in the sera was quantified at multiple time point using an anti-human Fc specific ELISA. Left graph, data are represented in nanomolar concentration. Right graph, area under the curve was calculated for each construction to define the in vivo drug exposure. Data are mean +/− SEM of 2-4 mice/group.

FIG. 14: Anti PD-1*1/protX*1 demonstrated higher pharmacokinetics in vivo than anti PD1*2/ProtX*1 molecules. C57BL/6 mice were intravenously injected with one dose (34 nmol/kg) of anti PD-1/IL-7, Anti PD-1/IL21, Anti PD-1/IL-2, Anti PD-1/IL-15 molecules constructed with an Anti PD-1*1 or anti PD-1*2 backbone. Antibodies concentration in the sera was quantified at multiple time points using an anti-human Fc specific ELISA. Area under the curve (AUC) was calculated for each construction to define the in vivo drug exposure. Each curve represents one type of molecule.

FIG. 15: Pharmacokinetics study of individual anti PD1*2/ProtX*1 molecules. Data represent pharmacokinetic and AUC of the individual constructions of anti PD-1/IL-7, Anti PD-1/IL21, Anti PD-1/IL-2, Anti PD-1/IL-15 molecules constructed with an Anti PD-1*1 or anti PD-1*2 backbone and one or 2 fused protx. FIG. 15. Anti PD-1/IL-7 wt FIG. 15B. Anti PD-1/IL-21 FIG. 15C. Anti PD-1/IL-2, FIG. 15D. Anti PD-1/IL-15. Data are mean +/− SEM of comprise 1-4 mice/group of independent experiment.

FIG. 16: Pharmacokinetics study in mice after a single injection of anti PD-1 alone (Anti PD-1*1 and Anti PD-1*2). C57 BL6 Mice were injected with one dose (34 nmol/kg) of anti PD-1 antibody constructed with one anti PD-1 valency (anti PD-1*1 , ▪ dashed line) or 2 anti PD-1 valency (anti PD-1*2 ●, plain line) (FIG. 16A) Intravenous injection and (FIG. 16B) Intraperitoneal injection. Antibodies concentration in the sera was quantified at multiple time points using an anti-human Fc specific ELISA. Data are represented in nM concentration.

FIG. 17: Anti PD-1*1 IL7v*1 molecules significantly promote T cell proliferation in vivo. Mice were intraperitoneally injected with one dose (34 nM/kg) of anti PD-1 IL-7v molecule (anti PD-1*2 IL7v*1, anti PD-1*1 IL7v*1, anti PD-1*1 IL7v*2, an anti PD-1*2 IL7wt*1, an anti PD-1*1 or, an anti PD -*2). On Day 4, tumor and blood were collected, and T cells were stained with ki67 proliferation marker in different T cell subpopulation. KI67 percentage was quantified in the CD3 CD4+ and CD3 CD8+ populations- in the blood (FIG. 17A and 17B) and in the intratumoral TCF1+ stem-like CD8 T cells (CD45/CD3/CD8/CD44/TCF1+/TOX−) (FIG. 17C). Statistical significance (*p<0.05) was calculated with one-way ANOVA test for multiple comparisons with control mice, n=3 to 8 mice per group.

FIG. 18: Anti PD-1*1 IL7*1 molecules demonstrated significant efficacy in the anti PD-1 resistant hepatocarcinoma orthotopic model. Humanized PD-1 KI immunocompetent mice were used for the experiment. Hepa1.6 hepatocarcinoma cells were orthotopically injected via the portal vein. On Day 4, mice were treated with 3 doses of PBS (negative control), anti PD-1*2, and anti PD-1*1 IL7*1. 2 independent experiments were performed. FIG. 18A. Survival of mice treated with the anti PD-1*1IL7v*1 versus Anti PD-1*2 and PBS treatments. FIG. 18B. Survival of mice treated with the anti PD-1*1IL7wt*1 versus Anti PD-1*2 and PBS treatments.

FIG. 19: Anti PD-1*1 IL7v*1 molecule demonstrated high efficacy in a mesothelioma orthotopic model. Humanized PD-1 KI immunocompetent mice were used for the experiment. AK7 mesothelioma cells were intraperitoneally injected. On Day 4, mice were treated with 3 doses of PBS (negative control), anti PD-1*2, anti PD-1*1 IL7v*1. FIG. 19A. Tumor burden measured by Bioluminescence. AK7 cells stably express luciferase allowing the in vivo quantification of bioluminescence. FIG. 19B. Survival of mice after treatments.

FIG. 20: Anti PD-1*1 IL7v*1 molecule abrogates suppressive function of Tregs to higher extent than IL-7 cytokine alone and anti PD-1*1 IL7wt*1. CD8+ effector T cells and autologous CD4+ CD25high CD127low Treg were isolated from peripheral blood of healthy donor, stained with cell proliferation dye (CPDe670 for CD8+ T cells). Treg/CD8+Teff were then co-cultured at ratio 1:1 on OKT3 coated plate (2 μg/mL) in presence or absence of rIL-7, anti PD-1*2, recombinant IL-7 cytokine, Anti PD-1*1 IL7WT*1 or anti PD-1*1 IL7v*1 (Anti PD-1*1 IL7W142H*) (0.12 nM) for 5 days. Proliferation of effector T cells was analyzed by cytofluorometry based on loss of CPD Marker. Data represent % suppressive activity of Treg analyzed using the formula 100−((% Teff cocultured with Tregs/% of Teff proliferation alone)*100). Data +/− SEM of n=4 donors of independent experiments. Statistical significance was One way ANOVA using Dunnett's test for multiple comparisons.

FIG. 21: Significant long-term monotherapy efficacy of anti PD-1*1 IL7v*1 molecule in anti PD-1 sensitive orthotopic model AK7 orthotopic model. hPD-1 KI mice were intraperitoneally injected with AK7 mesothelioma cells and treated with PBS (n=8 mice), anti PD-1*2 (n=8 mice), Anti PD-1*1 IL7v*1 (W142H*1) (n=14), anti PD-1*1 IL7WT *1 (A) Overall survival following treatment. Statistical significance was calculated with log-rank test (*p<0.05). (B) Anti PD-1*1 IL7v*1 induces a long-term memory response after tumor rechallenge. Mice cured by anti PD-1*1 IL7v*1 treatment (n=7) were rechallenged with AK7 mesothelioma cells via intraperitoneal injection (3e6 cells). As control, a group of naïve mice (n=3) was also injected to verify tumor load and growth. Graph represents luciferase transduced AK7 tumor cell growth as quantified by Bioluminescence following intraperitoneal injection of D-luciferin (150 mg/kg and analysis using bioimager) (mean +/− SEM).

FIG. 22: Preclinical efficacy of anti PD-1*1 IL7v*1 molecule in hepatocarcinoma orthotopic model. Following Hepa 1.6 tumor inoculation, Mice were treated on Day 4/6 and 8 with PBS, Anti PD-1, anti PD-1*1 IL7v*1 (Anti PD-1*1 IL7W142H*1) or anti PD-1*1 IL7wt*1. Overall survival obtained for 3 independent experiments and combined for illustration. PBS (n=23); Anti PD-1*2 (n=26), Isotype-IL-7 (n=14), anti PD-1*1 IL7v*1 (n=20) and anti PD-1*1IL7wt*1 (n=19). Statistical significance p<0.05 was calculated with Log Rank test.

FIG. 23: Gene signature after treatment with anti PD-1*1 IL7v*1 molecule in vivo showing increase in stem-like memory CD8 T cell subset into the tumor microenvironment. Following Hepa 1.6 tumor inoculation, Mice were treated on Day 4/6 and 8 with PBS, Anti PD-1 (34.3 nmol/kg) or anti PD-1*1 IL7v*1 or anti PD-1*1 IL7wt *1 (34.3 nmol/kg) (n=4 per group). On Day 10, tumor were collected, and gene expression was analyzed with Nanostring Pancancer immune panel (A) Heatmap representation of the gene differentially expressed (DEG) between PBS, Anti PD-1 and anti-PD-1*1IL7v*1 group with STRING protein-protein network analysis of common upregulated genes between anti PD-1 and BICKI IL7v treatments;. (B and C) Gene signature enrichment of early activated T cells versus exhausted T cells. Gene signatures of Exhausted T cells and Naïve like/Stem like memory T cells signature was adapted from Andreatta et al (Nature comm 2021, 12, 2965) Naïve-like/Stem-like memory Tcells (TCF7, CCR7, SELL, IL7R) and exhausted CD8 T cell score (LAG3, PRF1, CD8A, HAVRC2, GZMB, CD8B1, KLRD1, TNFRSF9, TIGIT, CTSW, CCL4, CD63, IFNG, CXCR6, FASL, CSF1).

FIG. 24: anti PD-1*1 IL7v*1 molecule induced proliferation of stem-like memory CD8 T cell (TCF1+) subset into the tumor microenvironment. (A,B and C) Following Hepa 1.6 tumor inoculation, mice were treated on Day 4/6 and 8 with PBS, Anti PD-1 (34.3 nmol/kg) or anti PD-1*1 IL7v*1 or anti PD-1*1 IL7wt *1 (34.3 nmol/kg) (n=4 per group). On Day 10, tumors were collected T cells and were stained for flow cytometry analysis for expression of CD3/CD8/CD44 marker and TCF1/TOX factors. (A) Percentage of the CD4, CD8, and Treg subpopulation into the tumor microenvironment (B) Percentage of CD44+ CD8+ activated T cells expressing TCF1+/−TOX markers (C) Proliferation of CD44+ CD8+ activated T cells expressing TCF1+/−TOX markers as measured by % of KI67 marker in Hepa1.6 model (D) in the MC38 subcutaneous model, Proliferation of CD44+ CD8+ activated T cells expressing TCF1+/−TOX markers was measured by % of KI67 marker after treatment (anti PD-1*2 anti PD-1*1 IL7v*1 (W142H*1))

FIG. 25: Anti PD-1*1 IL7v*1 maintains survival of chronically stimulated T cells (A) and induced proliferation of stem-like T cells in vitro (B). NXG immunodeficient mice were subcutaneously injected with MDA-MB231 Breast cancer cells carcinoma cells (3e6 cells), humanized with human PBMCs intraperitoneally on Day 8 (3e6 cells), then treated in i.p. with PBS, Anti PD-1*2 or anti PD-1*1 IL7v*1 on day 12, 15 and 18 post tumor inoculation. Data mean +/− SEM N=3 to 4 mice per group.

FIG. 26: Monotherapy efficacy of anti PD-1*1 IL7v*1 molecule in Breast cancer cells humanized mouse model. NXG immunodeficient mice were subcutaneously injected with MDA-MB231 Breast cancer cells carcinoma cells (3e6 cells), humanized with human PBMCs intraperitoneally on Day 8 (3e6 cells), then treated in i.p. with PBS, Anti PD-1*2 or anti PD-1*1 IL7v*1 on day 12, 15 and 18 post tumor inoculation. Data mean +/− SEM N=3 to 4 mice per group and per donor

FIG. 27: Monotherapy efficacy of anti PD-1*1 IL7v*1 molecule in Lung cancer A549 humanized mouse mode. NXG immunodeficient mice were subcutaneously injected with A549 lung carcinoma cells, humanized with human PBMCs intraperitoneally on Day 21 (10e6 cells), then treated in i.p. with PBS, Anti PD-1*2 or Anti PD-1*1 IL7v*1 on day 25, 28, 31 and 34 post tumor inoculation. (A) A549 Tumor growth n=5 mice per group, mean +/− SEM. Statistical significance **p<0.05 was calculated using Dunnett's multiple comparisons test by comparing anti PD-1*1 IL7v*1 versus anti PD-1*2 groups. (B) human IFNg secretion quantified by ELISA in the sera of mice collected on Day 35 (n=2-5 mice per group).

FIG. 28. Anti PD-1*1 IL7v*1 demonstrated a better pharmacokinetic profile compared to Anti PD-1*1 IL7wt*1 molecules and induced proliferation of CD8 T cells in vivo. Animals were intravenously injected with one dose of anti PD-1*1 IL-7v*1 (Anti PD-1*1 IL7 W142H*1) or anti PD-1*1 IL7wt*1 at 0.8 mg/kg (n=1 cyno), 4.01 mg/kg (n=1 cyno), 25 mg/kg (n=1 cyno) (A) Drug concentrations in the sera of animals were quantified by MSD immunoassay. (B) CD8 T cell proliferation measurement as assessed by flow cytometry in peripheral blood T cells following injection of the anti PD-1*1 IL7v*1 antibodies at different doses. (n=1 cyno per dose).

FIG. 29. Anti PD-1*1 IL7v*1 constructed with IgG1 N297A isotype or a IgG1 N297A LALA PG isotype demonstrated similar efficacy to activate pSTAT5. Stimulated human PBMCs were treated with anti PD-1*1 IL7v*1 constructed with an IgG1 N297A isotype or an IgG1 LALA P329G mutation at different doses. IL-7R signaling activation was measured with intracellular pSTAT5 staining into CD4 and CD8 T cells and analyzed by flow cytometry.

FIG. 30. Anti PD-1*1/protX*1 demonstrated absence of in vivo toxicity. C57BL/6 mice were intraperitoneally injected with either PBS, or a single (×1) or repeated (×3) injection every 2 days of Anti PD-1*1/IL7W142H*1 (A and B) or Anti PD-1*1/IL2*1 (C) antibodies. Mice were weighed every day and data were normalized to their initial weight prior molecule injection (Day 0=100%). (A) Anti PD-1/IL7 W14H*1 molecule injected one time at 20 mg/kg or three times at 5 mg/kg every 2 days. (B) Anti PD-1/IL7 W14H*1 molecule injected with escalating high dose one time at 50 or 100 mg/kg or three times at 20 mg/kg every 2 days. (C) Anti PD-1/IL-2*1 molecules injected one time at 20 mg/kg or three times at 5 mg/kg every 2 days.

### DETAILED DESCRIPTION OF THE INVENTION

### Introduction

The present invention relates to a bifunctional molecule having a particular scaffold and comprising a single monovalent antigen binding domain that binds a target specifically expressed on immune cells surface and a single immuno-stimulating cytokine. This scaffold is essentially made of a dimeric Fc domain, a single monovalent antigen binding domain that binds a target specifically expressed on immune cells surface linked at the N terminal end of one monomer of the Fc domain and a single immuno-stimulating cytokine linked at the C terminal end of the monomer of the Fc domain or the light chain when the antigen binding domain comprises a heavy variable chain and a light variable chain. These novel bifunctional molecules present, among other advantages, an improved pharmacokinetic profile, and a better productivity.

### Definitions

In order that the present invention may be more readily understood, certain terms are defined hereafter. Additional definitions are set forth throughout the detailed description.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art.

As used herein, the terms “wild type interleukin-7”, “wt-IL-7” and “wt-IL7” refers to a mammalian endogenous secretory glycoprotein, particularly IL-7 polypeptide. For example, wt-IL-7 refers to an amino acid sequence of a recombinant or non-recombinant polypeptide having an amino acid sequence of: i) a native or naturally-occurring IL-7 polypeptide, ii) a biologically active fragment of an IL-7 polypeptide, iii) a biologically active polypeptide analog of an IL-7 polypeptide, or iv) a biologically active IL-7 polypeptide. The IL-7 can comprise its peptide signal or be devoid of it. Alternative designations for this molecule are “pre-B cell growth factor” and “lymphopoietin-1”. Preferably, the term “wt-IL-7” refers to human IL-7 (wth-IL7). For example, the human wt-IL-7 amino acid sequence is about 152 amino acids (in absence of signal peptide) and has a Genbank accession number of NP_000871.1, the gene being located on chromosome 8q12-13. Human IL-7 is for example described in UniProtKB - P13232.

As used herein, the term “antibody” describes a type of immunoglobulin molecule and is used in its broadest sense. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass. The heavy-chain constant domains that correspond to the different classes of immunoglobulins are called alpha, delta, epsilon, gamma, and mu, respectively. Unless specifically noted otherwise, the term “antibody” includes intact immunoglobulins and “antibody fragment” or “antigen binding fragment” (such as Fab, Fab′, F(ab′)2, Fv), single chain (scFv), mutants thereof, molecules comprising an antibody portion, diabodies, linear antibodies, single chain antibodies, and any other modified configuration of the immunoglobulin molecule that comprises an antigen recognition site of the required specificity, including glycosylation variants of antibodies, amino acid sequence variants of antibodies. Preferably, the term antibody refers to a humanized antibody.

An “antibody heavy chain” as used herein, refers to the larger of the two types of polypeptide chains present in antibody conformations. The CDRs of the antibody heavy chain are typically referred to as “HCDR1”, “HCDR2” and “HCDR3”. The framework regions of the antibody heavy chain are typically referred to as “HFR1”, “HFR2”, “HFR3” and “HFR4”.

An “antibody light chain,” as used herein, refers to the smaller of the two types of polypeptide chains present in antibody conformations; κ and λ light chains refer to the two major antibody light chain isotypes. The CDRs of the antibody light chain are typically referred to as “LCDR1”, “LCDR2” and “LCDR3”. The framework regions of the antibody light chain are typically referred to as “LFR1”, “LFR2”, “LFR3” and “LFR4”.

As used herein, an “antigen-binding fragment” or “antigen-binding domain” of an antibody means a part of an antibody, i.e. a molecule corresponding to a portion of the structure of the antibody of the invention, that exhibits antigen-binding capacity for a particular antigen, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragment that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies. Antigen-binding fragments of antibodies are fragments which comprise their hypervariable domains designated CDRs (Complementary Determining Regions) or part(s) thereof.

As used herein, the term “humanized antibody” is intended to refer to antibodies in which CDR sequences derived from the germline of another mammalian species, such as a mouse, have been grafted onto human framework sequences (e.g. chimeric antibodies that contain minimal sequence derived from a non-human antibody). A “humanized form” of an antibody, e.g., a non-human antibody, also refers to an antibody that has undergone humanization. A humanized antibody is generally a human immunoglobulin (recipient antibody) in which residues from one or more CDRs are replaced by residues from at least one CDR of a non-human antibody (donor antibody) while maintaining the desired specificity, affinity, and capacity of the original antibody. Additional framework region modifications may be made within the human framework sequences. Preferably humanized antibody has a T20 humanness score greater than 80%, 85% or 90%. “Humanness” of an antibody can for example be measured using the T20 score analyzer to quantify the humanness of the variable region of antibodies as described in Gao S H, Huang K, Tu H, Adler A S. BMC Biotechnology. 2013: 13:55 or via a web-based tool to calculate the T20 score of antibody sequences using the T20 Cutoff Human Databases: http://abAnalyzer.lakepharma.com.

By “chimeric antibody” is meant an antibody made by combining genetic material from a nonhuman source, preferably such as a mouse, with genetic material from a human being. Such antibody derives from both human and non-human antibodies linked by a chimeric region. Chimeric antibodies generally comprise constant domains from human and variable domains from another mammalian species, reducing the risk of a reaction to foreign antibodies from a non-human animal when they are used in therapeutic treatments.

As used herein, the terms “fragment crystallizable region” “Fc region” or “Fc domain” are interchangeable and refers to the tail region of an antibody that interacts with cell surface receptors called Fc receptors. The Fc region or domain is typically composed of two domains, optionally identical, derived from the second and third constant domains of the antibody's two heavy chains (i.e. CH2 and CH3 domains). Portion of the Fc domain refers to the CH2 or the CH3 domain. Optionally, the Fc region or domain may optionally comprise all or a portion of the hinge region between CH1 and CH2. Accordingly, the Fc domain may comprise the hinge, the CH2 domain and the CH3 domain. Optionally, the Fc domain is that from IgG1, IgG2, IgG3 or IgG4, optionally with IgG1 hinge-CH2-CH3 and IgG4 hinge-CH2-CH3.

In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat.

By “amino acid change” or “amino acid modification” is meant herein a change in the amino acid sequence of a polypeptide. “Amino acid modifications” include substitution, insertion and/or deletion in a polypeptide sequence. By “amino acid substitution” or “substitution” herein is meant the replacement of an amino acid at a particular position in a parent polypeptide sequence with another amino acid. By “amino acid insertion” or “insertion” is meant the addition of an amino acid at a particular position in a parent polypeptide sequence. By “amino acid deletion” or “deletion” is meant the removal of an amino acid at a particular position in a parent polypeptide sequence. The amino acid substitutions may be conservative. A conservative substitution is the replacement of a given amino acid residue by another residue having a side chain (“R-group”) with similar chemical properties (e.g., charge, bulk and/or hydrophobicity). As used herein, “amino acid position” or “amino acid position number” are used interchangeably and refer to the position of a particular amino acid in an amino acids sequence, generally specified with the one letter codes for the amino acids. The first amino acid in the amino acids sequence (i.e. starting from the N terminus) should be considered as having position 1.

A conservative substitution is the replacement of a given amino acid residue by another residue having a side chain (“R-group”) with similar chemical properties (e.g., charge, bulk and/or hydrophobicity). In general, a conservative amino acid substitution will not substantially change the functional properties of a protein. Conservative substitutions and the corresponding rules are well-described in the state of the art. For instance, conservative substitutions can be defined by substitutions within the groups of amino acids reflected in the following tables:

TABLE A
Amino Acid Residue

| Amino acid groups | Amino acid residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

TABLE B
Alternative Conservative Amino Acid Residue Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE C
Further Alternative Physical and Functional
Classifications of Amino Acid Residues

| | |
|---|---|
| Alcohol group-containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Small residues | A, C, D, G, N, P, S, T, and V |
| Very small residues | A, G, and S |
| Residues involved in turn | A, C, D, E, G, H, K, N, Q, R, S, |
| formation | P, and T |
| Flexible residues | E, Q, T, K, S, G, P, D, E, and R |

As used herein, the “sequence identity” between two sequences is described by the parameter “sequence identity”, “sequence similarity” or “sequence homology”. For purposes of the present invention, the “percentage identity” between two sequences (A) and (B) is determined by comparing the two sequences aligned in an optimal manner, through a window of comparison. Said alignment of sequences can be carried out by well-known methods in the art, for example, using the algorithm for global alignment of Needleman-Wunsch. Protein analysis software matches similar sequences using measures of similarity assigned to various substitutions, deletions and other modifications, including conservative amino acid substitutions. Once the total alignment is obtained, the percentage of identity can be obtained by dividing the full number of identical amino acid residues aligned by the full number of residues contained in the longest sequence between the sequence (A) and (B). Sequence identity is typically determined using sequence analysis software. For comparing two amino acid sequences, one can use, for example, the tool “Emboss needle” for pairwise sequence alignment of proteins providing by EMBL-EBI and available on: www.ebi.ac.uk/Tools/services/web/toolform.ebi?tool=emboss_needle&context=protein, for example using default settings: (I) Matrix: BLOSUM62, (ii) Gap open: 10, (iii) gap extend: 0.5, (iv) output format: pair, (v) end gap penalty: false, (vi) end gap open: 10, (vii) end gap extend: 0.5.

Alternatively, Sequence identity can also be typically determined using sequence analysis software Clustal Omega using the HHalign algorithm and its default settings as its core alignment engine. The algorithm is described in Söding, J. (2005) ‘Protein homology detection by HMM-HMM comparison’. Bioinformatics 21, 951-960, with the default settings.

The terms “derive from” and “derived from” as used herein refers to a compound having a structure derived from the structure of a parent compound or protein and whose structure is sufficiently similar to those disclosed herein and based upon that similarity, would be expected by one skilled in the art to exhibit the same or similar properties, activities and utilities as the claimed compounds.

As used herein, a “pharmaceutical composition” refers to a preparation of one or more of the active agents, such as comprising a bifunctional molecule according to the invention, with optional other chemical components such as physiologically suitable carriers and excipients. The purpose of a pharmaceutical composition is to facilitate administration of the active agent to an organism. Compositions of the present invention can be in a form suitable for any conventional route of administration or use. In one aspect, a “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. An “acceptable vehicle” or “acceptable carrier” as referred to herein, is any known compound or combination of compounds that are known to those skilled in the art to be useful in formulating pharmaceutical compositions.

“An effective amount” or a “therapeutic effective amount” as used herein refers to the amount of active agent required to confer therapeutic effect on the subject, either alone or in combination with one or more other active agents, e.g. the amount of active agent that is needed to treat the targeted disease or disorder, or to produce the desired effect. The “effective amount” will vary depending on the agent(s), the disease and its severity, the characteristics of the subject to be treated including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

As used herein, the term “medicament” refers to any substance or composition with curative or preventive properties against disorders or diseases.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain aspects, such a term refers to the improvement or eradication of a disease, a disorder, an infection or symptoms associated with it. In other aspects, this term refers to minimizing the spread or the worsening of cancers. Treatments according to the present invention do not necessarily imply 100% or complete treatment. Rather, there are varying degrees of treatment of which one of ordinary skill in the art recognizes as having a potential benefit or therapeutic effect. Preferably, the term “treatment” refers to the application or administration of a composition including one or more active agents to a subject who has a disorder/disease.

As used herein, the terms “disorder” or “disease” refer to the incorrectly functioning organ, part, structure, or system of the body resulting from the effect of genetic or developmental errors, infection, poisons, nutritional deficiency or imbalance, toxicity, or unfavorable environmental factors. Preferably, these terms refer to a health disorder or disease e.g. an illness that disrupts normal physical or mental functions. More preferably, the term disorder refers to immune and/or inflammatory diseases that affect animals and/or humans, such as cancer.

“Immune cells” as used herein refers to cells involved in innate and adaptive immunity for example such as white blood cells (leukocytes) which are derived from hematopoietic stem cells (HSC) produced in the bone marrow, lymphocytes (T cells, B cells, natural killer (NK) cells and Natural Killer T cells (NKT) and myeloid-derived cells (neutrophil, eosinophil, basophil, monocyte, macrophage, dendritic cells). In particular, the immune cell can be selected in the non-exhaustive list comprising B cells, T cells, in particular CD4+ T cells and CD8+ T cells, NK cells, NKT cells, APC cells, dendritic cells and monocytes. “T cell” as used herein includes for example CD4+ T cells, CD8+ T cells, T helper 1 type T cells, T helper 2 type T cells, T helper 17 type T cells and inhibitory T cells.

As used herein, the term “T effector cell”, “T eff” or “effector cell” describes a group of immune cells that includes several T cells types that actively respond to a stimulus, such as co-stimulation. It particularly includes T cells which function to eliminate antigen (e.g., by producing cytokines which modulate the activation of other cells or by cytotoxic activity). It notably includes CD4+, CD8+, cytotoxic T cells and helper T cells (Th1 and Th2).

As used herein, the term “regulatory T cell”, Treg cells” or “T reg” refers to a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FOXP3, and CD25 and are thought to be derived from the same lineage as naïve CD4 cells.

The term “exhausted T cell” refers to a population of T cell in a state of dysfunction (i.e. “exhaustion”). T cell exhaustion is characterized by progressive loss of function, changes in transcriptional profiles and sustained expression of inhibitory receptors. Exhausted T cells lose their cytokines production capacity, their high proliferative capacity and their cytotoxic potential, which eventually leads to their deletion. Exhausted T cells typically indicate higher levels of CD43, CD69 and inhibitory receptors combined with lower expression of CD62L and CD127.

The term “effector memory stem like T cell” refers to a subset of tumor-reactive intra-tumoral T cells bearing hallmarks of exhausted cells and central memory cells, including expression of the checkpoint protein PD-1 and the transcription factor Tcf1. These cells can be called Tcf1⁺PD-1⁺CD8⁺ T cells. These cells reside in the tumor microenvironment and are critical for immune control of cancer promoted by immunotherapy. They are critical for maintaining the T cell response during chronic viral infection and cancer, and provide the proliferative burst seen after PD-1 immunotherapy. These cells undergo a slow self-renewal and also give rise to the more terminally differentiated exhausted CD8 T cells. These cells and their characteristics are further defined in the following articles, the disclosure thereof being incorporated herein by reference: Siddiqui et al, 2019, Immunity, 50, 195-211; and Jadhav et al, 2019, PNAS, 116, 14113-14118).

The term “immune response” refers to the action of, for example, lymphocytes, antigen presenting cells, phagocytic cells, granulocytes, and soluble macromolecules produced by the above cells or the liver (including antibodies, cytokines, and complements) that results in selective damage to, destruction of, or elimination from the human body of invading pathogens, cells or tissues infected with pathogens, cancerous cells, or, in cases of autoimmunity or pathological inflammation, normal human cells or tissues. The term “antagonist” as used herein, refers to a substance that blocks or reduces the activity or functionality of another substance. Particularly, this term refers to an antibody that binds to a cellular receptor (e.g. PD-1) as a reference substance (e.g. PD-L1 and/or PD-L2), preventing it from producing all or part of its usual biological effects (e.g. the creation of an immune suppressive microenvironment). The antagonist activity of a humanized antibody according to the invention may be assessed by competitive ELISA.

The term “agonist” as used herein, refers to a substance that activates the functionality of an activating receptor. Particularly, this term refers to an antibody that binds to a cellular activating receptor as a reference substance, and have at least partially the same effect of the biologically natural ligand (e.g. inducing the activatory effect of the receptor).

Pharmacokinetics (PK) refers to the movement of drugs through the body, whereas pharmacodynamics (PD) refers to the body's biological response to drugs. PK describes a drug's exposure by characterizing absorption, distribution, bioavailability, metabolism, and excretion as a function of time. PD describes drug response in terms of biochemical or molecular interactions. PK and PD Analyses are used to characterize drug exposure, predict and assess changes in dosage, estimate rate of elimination and rate of absorption, assess relative bioavailability/bioequivalence of a formulation, characterize intra- and inter-subject variability, understand concentration-effect relationships, and establish safety margins and efficacy characteristics. By “improving PK” it is meant that one of the above characteristics is improved, for example, such as an increased half-life of the molecule, in particular a longer serum half-life of the molecule when injected to a subject.

As used herein, the terms “pharmacokinetics” and “PK” are used interchangeably and refer to the fate of compounds, substances or drugs administered to a living organism. Pharmacokinetics particularly comprise the ADME or LADME scheme, which stands for Liberation (i.e. the release of a substance from a composition), Absorption (i.e. the entrance of the substance in blood circulation), Distribution (i.e. dispersion or dissemination of the substance through the body) Metabolism (i.e. transformation or degradation of the substance) and Excretion (i.e. the removal or clearance of the substance from the organism). The two phases of metabolism and excretion can also be grouped together under the title elimination. Different pharmacokinetics parameters can be monitored by the man skilled in the art, such as elimination half-life, elimination constant rate, clearance (i.e. the volume of plasma cleared of the drug per unit time), Cmax (Maximum serum concentration), and Drug exposure (determined by Area under the curve, see Scheff et al, Pharm Res. 2011 May;28(5): 1081-9) among others.

As used herein, the term “isolated” indicates that the recited material (e.g., antibody, polypeptide, nucleic acid, etc.) is substantially separated from, or enriched relative to, other materials with which it occurs in nature. Particularly, an “isolated” antibody is one which has been identified and separated and/or recovered from a component of its natural environment.

The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually.

The term “a” or “an” can refer to one of or a plurality of the elements it modifies (e.g., “a reagent” can mean one or more reagents) unless it is contextually clear either one of the elements or more than one of the elements is described.

The term “about” as used herein in connection with any and all values (including lower and upper ends of numerical ranges) means any value having an acceptable range of deviation of up to +/−10% (e.g., +/−0.5%, +/−1%, +/−1.5%, +/−2%, +/−2.5%, +/−3%, +/−3.5%, +/−4%, +/−4.5%, +/−5%, +/−5.5%, +/−6%, +/−6.5%, +/−7%, +/−7.5%, +/−8%, +/−8.5%, +/−9%, +/−9.5%). The use of the term “about” at the beginning of a string of values modifies each of the values (i.e. “about 1, 2 and 3” refers to about 1, about 2 and about 3). Further, when a listing of values is described herein (e.g. about 50%, 60%, 70%, 80%, 85% or 86%) the listing includes all intermediate and fractional values thereof (e.g., 54%, 85.4%).

### Bifunctional Molecules

The present invention relates to a bifunctional molecule having a scaffold with improved properties.

More particularly, the present invention relates to a bifunctional molecule having a particular scaffold and comprising a single monovalent antigen binding domain that binds a target specifically expressed on immune cells surface and a single immuno-stimulating cytokine. This scaffold is essentially made of a dimeric Fc domain, a single monovalent antigen binding domain that binds a target specifically expressed on immune cells surface linked at the N terminal end of one monomer of the Fc domain and either i) a single immuno-stimulating cytokine linked at the C terminal end of the same monomer of the Fc domain, and optionally peptide linkers, or ii) the single monovalent antigen binding domain comprises a heavy variable chain and a light variable chain and the single immuno-stimulating cytokine is linked at the C terminal end of the light chain of said antigen binding domain.

In a particular aspect, the bifunctional molecule comprises a first monomer comprising an antigen-binding domain covalently linked to a first Fc chain optionally via a peptide linker, said first Fc chain being covalently linked to the immuno-stimulating cytokine, optionally via a peptide linker, and a second monomer comprising a complementary second Fc chain, devoid of antigen-binding domain and of immuno-stimulating cytokine, said first and second Fc chains forming a dimeric Fc domain.

In an alternative aspect, the bifunctional molecule comprises a first monomer comprising an antigen-binding domain covalently linked to a first Fc chain optionally via a peptide linker, a second monomer comprising a complementary second Fc chain, devoid of antigen-binding domain and of immuno-stimulating cytokine, said first and second Fc chains forming a dimeric Fc domain, and the single monovalent antigen binding domain comprises a heavy variable chain and a light variable chain and the single immuno-stimulating cytokine is linked at the C terminal end of the light chain of said antigen binding domain.

Accordingly, two monomers comprise each one a Fc chain, the Fc chains being able to form a dimeric Fc domain. In one aspect, the dimeric Fc fusion protein is a homodimeric Fc domain. In another aspect, the dimeric Fc fusion protein is a heterodimeric Fc domain.

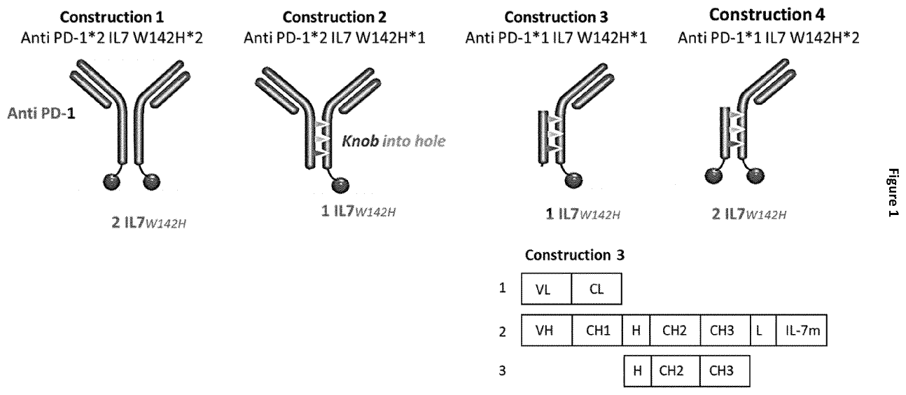

More particularly, when the dimeric Fc domain is a heterodimeric Fc domain, the bifunctional molecule comprises a first monomer comprising an antigen-binding domain covalently linked to the N-terminal end of the first heterodimeric Fc chain optionally via a peptide linker, said first heterodimeric Fc chain being covalently linked by its C-terminal end to an immuno-stimulating cytokine, optionally via a peptide linker, and a second monomer comprising a complementary second heterodimeric Fc chain devoid of antigen-binding domain and of immuno-stimulating cytokine. Optionally, said second monomer comprising a complementary second heterodimeric Fc chain is devoid of any other functional moiety. Still more particularly, the bifunctional molecule comprises a first monomer comprising an antigen-binding domain covalently linked via C-terminal end to N-terminal end of a first heterodimeric Fc chain optionally via a peptide linker, said first heterodimeric Fc chain being covalently linked by its C-terminal end to the N-terminal end of the immuno-stimulating cytokine, optionally via a peptide linker, and a second monomer comprising a complementary second heterodimeric Fc chain devoid of antigen-binding domain and of immuno-stimulating cytokine, preferably devoid of any other functional moiety. Such a bifunctional molecule is illustrated for example as “construct 3” in FIG. 1, with IL-7 as illustration of an immuno-stimulating cytokine or as format C in FIG. 6.

Optionally, the single antigen-binding domain selected from the group consisting of a Fab, a Fab′, a scFV and a sdAb.

Accordingly, in one aspect, the bifunctional molecule according to the invention comprises or consists of:
(a) an antigen-binding domain that binds a target specifically expressed on immune cells surface, which comprises (i) one heavy chain with a first Fc chain, and (ii) one light chain,
(b) an immuno-stimulating cytokine, and
(c) a complementary second Fc chain,
wherein the immuno-stimulating cytokine is covalently linked, optionally via a peptide linker, preferably by its N-terminal end, to the C-terminal end of the first Fc chain. The first Fc chain and the second Fc chain form together a dimeric Fc domain.

In a particular aspect, the bifunctional molecule comprises
one antibody heavy chain including VH-CH1-hinge-CH2-CH3 linked at its C-terminal end to an immuno-stimulating cytokine,
one antibody light chain including VL-CL (constant light chain), the VH-CH1 moiety and the VL-CL moiety forming together an antigen binding domain that binds a target specifically expressed on immune cells surface, and
one Fc chain comprising CH2-CH3, optionally hinge-CH2-CH3, forming with the CH2-CH3 of the antibody heavy chain a dimeric Fc domain.

According to an alternative aspect, when the dimeric Fc domain is a heterodimeric Fc domain, the bifunctional molecule comprises a first monomer comprising an antigen-binding domain covalently linked to the N-terminal end of the first heterodimeric Fc chain optionally via a peptide linker, a second monomer comprising a complementary second heterodimeric Fc chain devoid of antigen-binding domain and of immuno-stimulating cytokine, and said antigen-binding domain comprises a heavy variable chain and a light variable chain and the immuno-stimulating cytokine is linked, optionally via a peptide linker, at the C terminal end of the light chain of said antigen-binding domain. Optionally, said immuno-stimulating cytokine is linked, optionally via a peptide linker, at the C terminal end of the light chain of said antigen-binding domain by its N terminal end.

Accordingly, in this aspect, the bifunctional molecule according to the invention comprises or consists of:
(a) an antigen-binding domain that binds a target specifically expressed on immune cells surface, which comprises (i) one heavy chain with a first Fc chain, and (ii) one light chain,
(b) an immuno-stimulating cytokine, and
(c) a complementary second Fc chain,
wherein the immuno-stimulating cytokine is covalently linked, optionally via a peptide linker, preferably by its N-terminal end, to the C-terminal end of the light chain. The first Fc chain and the second Fc chain form together a dimeric Fc domain.

In a particular aspect, the bifunctional molecule comprises
one antibody heavy chain including VH-CH1-hinge-CH2-CH3,
one antibody light chain including VL-CL (constant light chain) linked at its C-terminal end to an immuno-stimulating cytokine, the VH-CH1 moiety and the VL-CL moiety forming together an antigen binding domain that binds a target specifically expressed on immune cells surface, and
one Fc chain comprising CH2-CH3, optionally hinge-CH2-CH3, forming with the CH2-CH3 of the antibody heavy chain a dimeric Fc domain.

The immuno-stimulating cytokine, the antigen binding domain that binds a target specifically expressed on immune cells surface, the Fc domain and the optional linkers are as further defined below in any of the aspects.

### Immuno-Stimulating Cytokine

The immuno-stimulating cytokine is capable of stimulating or activating an immune cell. The immune cell can be selected in the non-exhaustive list comprising B cells, T cells, in particular CD4+ T cells and CD8+ T cells, NK cells, NKT cells, APC cells, dendritic cells and monocytes. In a preferred aspect, the immune cells are T cells, more specifically CD8+ T cells, effector T cells or exhausted T cells. In a particular preferred aspect, the immune cells are effector memory stem like T cells.

Preferably, the immuno-stimulating cytokine is selected from the group consisting of cytokines and chemokines. Particularly, the immuno-stimulating cytokine has a size comprised between 10 kDa and 50 kDa. Preferably, the immuno-stimulating cytokine is a peptide, a polypeptide or a protein. In one aspect, the immuno-stimulating cytokine is a non-antibody entity or portion.

For instance, the immuno-stimulating cytokine can be selected from: T-cell growth factors, in particular growth factors to increase number and repertoire of naive T cells, growth factors to increase the number of dendritic cells (DCs), agonists to activate DCs and other antigen-presenting cells (APCs), adjuvants to allow and augment cancer vaccines, agonists to activate and stimulate T cells, inhibitors of T-cell checkpoint blockade, T-cell growth factors to increase the growth and survival of immune T cells, agents to inhibit, block, or neutralize cancer cell and immune cell-derived immunosuppressive cytokine. In a particular aspect, the cytokine is able to activate and stimulate effector memory stem like T cell. The immuno-stimulating cytokine may be mutated or altered so that the biological activity is altered, e.g. the biological activity is increased, decreased or totally inhibited.

In a particular aspect, the immuno-stimulating cytokine is selected from the group consisting of IL-2 (IL being interleukin), IL-4, IL-5, IL-6, IL-12A, IL-12B, IL-13; IL-15, IL-18, IL-21, IL-23, IL-24, IFNα (interferon alpha), IFNα (interferon beta), BAFF, LTα and LTβ or a variant thereof having at least 80, 85, 90, 91, 92, 93, 94, 95, 96, 97, 98 or 99% of identity with the wildtype cytokine or having 1 to 10 modifications selected from the group consisting of addition, deletion, substitution and combinations thereof. The immuno-stimulating cytokine can also be IL-7.

In particular, the immuno-stimulating cytokine can be selected in the list of Table D below.

TABLE D
List of immunostimulating cytokines

| Name | Official name | Uniprot reference |
|---|---|---|
| IL-2 | Interleukin-2, IL-2 (T-cell growth factor, TCGF) | P60568 |
| IL-4 | Interleukin-4 (B-cell stimulatory factor 1, BSF-1, Binetrakin, | P05112 |
| | Lymphocyte stimulatory factor 1, Pitrakinra) | |
| IL-5 | Interleukin-5, B-cell differentiation factor I, Eosinophil differentiation | P05113 |
| | factor, T-cell replacing factor, TRF | |
| IL-6 | Interleukin-6, IL-6 (B-cell stimulatory factor 2, BSF-2) (CTL | P05231 |
| | differentiation factor, CDF) (Hybridoma growth factor) (Interferon | |
| | beta-2, IFN-beta-2) | |
| IL-7 | Interleukin-7 | P13232 |
| IL-12A | Interleukin-12 subunit alpha, IL-12A (Cytotoxic lymphocyte maturation | P29459 |
| | factor 35 kDa subunit, CLMF p35) (IL-12 subunit p35) (NK cell | |
| | stimulatory factor chain 1, NKSF1) | |
| IL-12B | Interleukin-12 subunit beta, IL-12B (Cytotoxic lymphocyte maturation | P29460 |
| | factor 40 kDa subunit, CLMF p40) (IL-12 subunit p40) (NK cell | |
| | stimulatory factor chain 2, NKSF2) | |
| IL-13 | Interleukin-13, | P35225 |
| IL-15 | Interleukin-15, IL-15 | P40933 |
| IL-18 | Interleukin-18 (Iboctadekin, Interferon gamma-inducing factor, IFN- | Q14116 |
| | gamma-inducing factor, Interleukin-1 gamma, IL-1 gamma) | |
| IL-21 | Interleukin-21, IL-21, Za11 | Q9HBE4 |
| IL-23 | Interleukin-23 subunit alpha, IL-23 subunit alpha, IL-23-A, Interleukin- | Q9NPF7 |
| | 23 subunit p19, IL-23p19 | |
| IL-24 | Interleukin-24, Melanoma differentiation-associated gene 7 protein, | Q13007 |
| | MDA-7, Suppression of tumorigenicity 16 protein | |
| IFNα | Interferon alpha including Interferon alpha-1 (i.e., Interferon alpha-D), | P01562, P01563, |
| | Interferon alpha-2, (i.e., Interferon alpha-A), Interferon alpha-4 (i.e., | P05014, P01570, |
| | Interferon alpha-4B, Interferon alpha-76, Interferon alpha-M), | P32881, P01567, |
| | Interferon alpha-14 (i.e., Interferon alpha-H, Interferon lambda-2-H), | P01571, P01569, |
| | Interferon alpha-8 (i.e., Interferon alpha-B, Interferon alpha-B2), | P05013, P01566, |
| | Interferon alpha-7 (i.e., Interferon alpha-J, Interferon alpha-J1), | P05015 |
| | Interferon alpha-17 (i.e., Interferon alpha-88, Interferon alpha-I, | |
| | Interferon alpha-T), Interferon alpha-5 (i.e., Interferon alpha-61, | |
| | Interferon alpha-G), Interferon alpha-6 (i.e., Interferon alpha-54, | |
| | Interferon alpha-K), Interferon alpha-10 (i.e., Interferon alpha-6L, | |
| | Interferon alpha-C), Interferon alpha-16 (i.e., Interferon alpha-WA) | |
| IFNβ | Interferon beta, Fibroblast interferon, | P01574 |
| BAFF | Tumor necrosis factor ligand superfamily member 13B, TNFSF13B, B- | Q9WU72 |
| | cell-activating factor, CD257 | |
| LTα | TNFB_HUMAN Lymphotoxin-alpha, LT-alpha (TNF-beta) (Tumor | P01374 |
| | necrosis factor ligand superfamily member 1) | |
| LTβ | Lymphotoxin-beta (LT-beta) Tumor necrosis factor C | Q06643 |

In a particular aspect, the immuno-stimulating cytokine is not IL-2 or a variant thereof.

Accordingly, the immuno-stimulating cytokine is selected from the group consisting of IL-2 (IL being interleukin), IL-4, IL-5, IL-6, IL-12A, IL-12B, IL-13; IL-15, IL-18, IL-21, IL-23, IL-24; IFNα (interferon alpha), IFNβ (interferon beta), BAFF, LTα, and LTβ, or a variant thereof having at least 80, 85, 90, 91, 92, 93, 94, 95, 96, 97, 98 or 99% of identity with the wildtype protein or the extracellular fragment thereof or having 1 to 10 modifications selected from the group consisting of addition, deletion, substitution and combinations thereof. The immuno-stimulating cytokine can also be IL-7.

In a particularly, the immuno-stimulating cytokine is selected from the group consisting of IL-2, IL-15 and IL-21 or a variant thereof having at least 80, 85, 90, 91, 92, 93, 94, 95, 96, 97, 98 or 99% of identity with the wildtype cytokine or having 1 to 10 modifications selected from the group consisting of addition, deletion, substitution and combinations thereof.

In a very specific aspect, the immuno-stimulating cytokine is IL-2 or a variant thereof having at least 80, 85, 90, 91, 92, 93, 94, 95, 96, 97, 98 or 99% of identity with the wildtype cytokine or having 1 to 10 modifications selected from the group consisting of addition, deletion, substitution and combinations thereof.

In a very specific aspect, the immuno-stimulating cytokine is IL-7, in particular an IL-7 having a sequence as set forth in SEQ ID NO: 1.

In a very specific aspect, the immuno-stimulating cytokine is IL-15 or a variant thereof having at least 80, 85, 90, 91, 92, 93, 94, 95, 96, 97, 98 or 99% of identity with the wildtype cytokine or having 1 to 10 modifications selected from the group consisting of addition, deletion, substitution and combinations thereof.

In a very specific aspect, the immuno-stimulating cytokine is IL-21 or a variant thereof having at least 80, 85, 90, 91, 92, 93, 94, 95, 96, 97, 98 or 99% of identity with the wildtype cytokine or having 1 to 10 modifications selected from the group consisting of addition, deletion, substitution and combinations thereof.

### IL-2 and IL-2 Variants

In a very specific aspect, the immuno-stimulating cytokine is Interleukin-2 (IL-2), preferably a human IL-2, for example as disclosed under the UniProt accession number P60568 or a mutant or variant thereof.

The IL-2 variant preferably has at least 80, 85, 90, 91, 92, 93, 94, 95, 96, 97, 98 or 99% of identity with the wildtype cytokine of SEQ ID NO: 87 or having 1 to 10 modifications selected from the group consisting of addition, deletion, substitution and combinations thereof with respect to the sequence of SEQ ID NO: 87.

IL-2 can be mutated in various ways to reduce its toxicity and/or increase its efficacy. Hu et al. (Blood 101, 4853-4861 (2003), US Pat. Publ. No. 2003/0124678) have substituted the arginine residue in position 38 of IL-2 by tryptophan to eliminate IL-2's vasopermeability activity. Shanafelt et al. (Nature Biotechnol 18, 1 197-1202 (2000)) have mutated asparagine 88 to arginine to enhance selectivity for T cells over NK cells. Heaton et al. (Cancer Res 53, 2597-602 (1993); U.S. Pat. No. 5,229,109) have introduced two mutations, Arg38Ala and Phe42Lys, to reduce the secretion of proinflammatory cytokines from NK cells. Gillies et al. (US Pat. Publ. No. 2007/0036752) have substituted three residues of IL-2 (Asp20Thr, Asn88Arg, and GlnI26Asp) that contribute to affinity for the intermediate-affinity IL-2 receptor to reduce VLS. Gillies et al. (WO 2008/0034473) have also mutated the interface of IL-2 with CD25 by amino acid substitution Arg38Trp and Phe42Lys to reduce interaction with CD25 and activation of Treg cells for enhancing efficacy. In one aspect, the immunotherapeutic agent is an IL-2 mutant for example as described in WO 2012/107417 or WO 2018/184964.

Optionally, the IL-2 variant may comprise one or several substitutions at positions of human IL-2 (without the signal peptide; SEQ ID NO: 87) selected from the group consisting of Q11, H16, L18, L19, D20, Q22, R38, F42, K43, Y45, E62, P65, E68, V69, L72, D84, S87, N88, V91, 192, T123, Q126, SI 27, 1129, and S130. Optionally, the IL-2 variant may comprise the substitution relative to human IL-2 (without the signal peptide; SEQ ID NO: 87) F42A or F42K. Optionally, the IL-2 variant may further comprise one or several substitutions relative to human IL-2 (without the signal peptide; SEQ ID NO: 87) selected from the group consisting of
R38A, R38D, R38E, E62Q, E68A, E68Q, E68K and E68R, and/or
H16E, H16D, D20N, M23A, M23R, M23K, S87K, S87A, D84L, D84N, D84V, D84H, D84Y, D84R, D84K, N88A, N88S, N88T, N88R, N88I, V91A, V91T, V91E, I92A, E95S, E95A, E95R, T123A, T123E, T123K, T123Q, Q126A, Q126S, Q126T, Q126E, SI 27 A, S127E, S127K, and S127Q, and/or
C125A.

Optionally, the IL-2 variant may comprise one of the following substitutions combination relative to human IL-2 (without the signal peptide; SEQ ID NO: 87): R38E and F42A; R38D and F42A; F42A and E62Q; R38A and F42K; R38E, F42A, and N88S; R38E, F42A, and N88A; R38E, F42A, and V91E; R38E, F42A, and D84H; H16D, R38E and F42A; H16E, R38E and F42A; R38E, F42A and Q126S; R38D, F42A and N88S; R38D, F42A and N88A; R38D, F42A and V91E; R38D, F42A, and D84H; H16D, R38D and F42A; H16E, R38D and F42A; R38D, F42A and Q126S; R38A, F42K, and N88S; R38A, F42K, and N88A; R38A, F42K, and V91E; R38A, F42K, and D84H; H16D, R38A, and F42K; H16E, R38A, and F42K; R38A, F42K, and Q126S; F42A, E62Q, and N88S; F42A, E62Q, and N88A; F42A, E62Q, and V91E; F42A, E62Q, and D84H; H16D, F42A, and E62Q; H16E, F42A, and E62Q; F42A, E62Q, and Q126S; R38E, F42A, and C125A; R38D, F42A , and C125A; F42A, E62Q, and C125A; R38A, F42K, and C125A; R38E, F42A, N88S, and C125A; R38E, F42A, N88A, and C125A; R38E, F42A, V91E, and C125A; R38E, F42A, D84H, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, V91E, and C125A; R38D, F42A, D84H, and C125A; H16D, R38D, F42A, and C125A; H16E, R38D, F42A, and C125A; R38D, F42A, C125A, and Q126S; R38A, F42K, N88S, and C125A; R38A, F42K, N88A, and C125A; R38A, F42K, V91E, and C125A; R38A, F42K, D84H, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, V91E, and C125A; F42A, E62Q, and D84H, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, V91E, and C125A; F42A, D84H, and C125A; H16D, F42A, and C125A; H16E, F42A, and C125A; F42A, C125A and Q126S; F42A, Y45A and L72G; and T3A, F42A, Y45A, L72G and C125A.

Optionally, the IL-2 variant may comprise one of the following substitutions relative to human IL-2 (without the signal peptide; SEQ ID NO: 87), in particular at least one of the substitutions selected in the group comprising K35E, K35A, R38A, R38E, R38N, R38F, R38S, R38L, R38G, R38Y, R38W, F42L, F42A, F42G, F42S, F42T, F42Q, F42E, F42N, F42D, F42R, F42K, K43E, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K; or a combination thereof, preferably the three substitutions F42A, Y45A and L72G.

Mutants of human IL-2 (hIL-2) with decreased affinity to CD25 may for example be generated by amino acid substitution at amino acid position 3, 35, 38, 42, 43, 45 or 72 or combinations thereof, corresponding to residues position of human IL-2 (without the signal peptide; SEQ ID NO: 87). Preferably, the mutant IL-2 is a human IL-2 molecule comprising the amino acid substitutions relative to human IL-2 (without the signal peptide; SEQ ID NO: 87) T3A, F42A, Y45A, L72G and/or C125A, preferably F42A, Y45A and L72G, more preferably T3A, F42A, Y45A, L72G and C125A, for example as disclosed in WO 2018/184964. Even more preferably, the immuno-stimulating cytokine is an IL-2 mutant having the substitutions relative to human IL-2 (without the signal peptide; SEQ ID NO: 87) F42A, Y45A and L72G, preferably T3A, F42A, Y45A, L72G and C125A.

### IL-12A and IL-12B Variants

In a particular aspect, the immuno-stimulating cytokine is a variant of IL-12A or IL-12B.

For instance, it could be a variant of IL-12A having one or several substitutions with respect to the wildtype IL-12A selected from the group consisting of N21D, Q35D, E38Q, D55Q, D55K, N71D, N71Q, L75A, N76D, E79Q, N85D, N85Q, L89A, F96A, M97A, L124A, M125A, Q130E, Q135E, N136D, E143Q, Q146E, N151D, N151K, E153K, E153Q, K158E, E162Q, E163Q, D165N, I171A, N195D, and N195Q. Optionally, the variant of IL-12A has one of the following substitutions combinations: N71D/N85D/N195D, N151D/E153Q, N151D/D165N, Q130E/N151D, N151D/K158E, E79Q/N151D, D55Q/N151D, N136D/N151D, N21D/N151D, E143Q/N151D, N71Q/N85Q, N71Q/N195Q, N85Q/N195Q, N71Q/N85Q/N195Q, N71D/N85D, N71D/N195D, and N85D/N195D.

For instance, it could be a variant of IL-12B having one or several substitutions with respect to the wildtype IL-12B selected from the group consisting of E59K, E59Q, D18N, D18K, E32Q, E33Q, D34N, D34K, Q42E, S43E, S43K, E45Q, Q56E, D62N, E73Q, D87N, K99E, K99Y, E100Q, N103D, N103Q, N113D, N113Q, Q144E, D161N, R159E, K163E, E187Q, N200D, N200Q, N218Q, Q229E, E235Q, C252S, Q256N, K258E, K260E, E262Q, K264E, N281D, N281Q, and E299Q. Optionally, the variant of IL-12B has one of the following substitutions combinations: N103D/N113D/N200D/N281D, Q42E/E45Q, E45Q/Q56E, Q42E/E59Q, Q56E/E59Q, Q42E/E45Q/Q56E, E45Q/Q56E/E59Q, E32Q/E59Q, D34N/E59K, D34N/E59K/K99E, D34K/E59K/K99E, E32Q/D34N/E59K/K99E, E32K/D34N/E59K/K99E, D34N/E59Q, E59Q/E187Q, S43E/E59Q, S43K/E49Q, E59Q/K163E, E59Q/K99E, E59Q/K258E, E59Q/K260E, E59K/K99E, D18K/E59K/K99E, E59K/K99E/K264E, E59K/K99Y, E59Y/K99Y, E59Y/K99E, E45K/E59K/K99E, E59K/K99E/Q144E, E59K/K99E/Q144K, E59K/K99E/R159E, E59K/K99E/K264E, D18K/E59K/K99E/K264E, DI8K/E59K/K99E/C252S, D18K/E59K/K99E/C252S/K264E, E59K/K99Y/C252S, E59K/K99E/C252S/K264E, E59K/K99E/C252S, N103D/N113D, N103D/N200D, N103D/N281D, N113D/N200D, N113D/N281D, N200D/N281D, N103D/N113D/N200D, N103D/N113D/N281D, N103D/N200D/N281D, N113D/N200D/N281D, N103Q/N113Q, N103Q/N200Q, N103Q/N281Q, N113Q/N200Q, N113Q/N281Q, N200Q/N281Q, N103Q/N113Q/N200Q, N103Q/N113Q/N281Q, N103Q/N200Q/N281Q, N103Q/N113Q/N200Q/N281Q, E59K/K99E/N103Q/C252S/K264E, N113Q/N200Q/N281Q, E59K/K99E/N113Q/C252S/K264E, E59K/K99E/N200Q/C252S/K264E, E59K/K99E/N281Q/C252S/K264E, E59K/K99E/N103Q/N113Q/C252S/K264E, E59K/K99E/N103Q/N200Q/C252S/K264E, E59K/K99E/N113Q/N200Q/C252S/K264E, E59K/K99E/N103Q/N281Q/C252S/K264E, E59K/K99E/N200Q/N281Q/C252S/K264E, E59K/K99E/N113Q/N281Q/C252S/K264E, E59K/K99E/N103Q/N113Q/N200Q/C252S/K264E, E59K/K99E/N103Q/N200Q/N281Q/C252S/K264E, E59K/K99E/N113Q/N200Q/N281Q/C252S/K264E, and E59K/K99E/N103Q/N113Q/N200Q/N281Q/C252S/K264E.

As mentioned herein the “/” refer to substitutions that are cumulative. Thus, by the mutation Q42E/E45Q, it is meant the following substitutions: Q42E and E45Q.

### IL-15 Variants

In a particular aspect, the immuno-stimulating cytokine is a variant of IL-15.

The IL-15 variant preferably has at least 80, 85, 90, 91, 92, 93, 94, 95, 96, 97, 98 or 99% of identity with the wildtype cytokine of SEQ ID NO: 88 or having 1 to 10 modifications selected from the group consisting of addition, deletion, substitution and combinations thereof with respect to the sequence of SEQ ID NO: 88.

Optionally, the IL-15 variant may comprise one or several substitutions at positions of human IL-15 (without the signal peptide; SEQ ID NO: 88) selected from the group consisting of N1D, V3I, V3M, V3R, N4D, D8N, D8A, K11L, K11M, K11R, D30N, D61N, E64Q, N65D, N71D, N71S, N72D, N72A, N72R, N72Y, S73I, N77A, N79D, N79E, N79S, Q108E, N112D, N112H, N112M and N112Y, preferably N4D, D61N, N65D, and Q108E.

Optionally, the IL-15 variant may comprise one of the following substitutions combination relative to human IL-15 (without the signal peptide; SEQ ID NO: 88): N4D/N65D, D30N/N65D, D30N/E64Q, D30N/E64Q/N65D, N1D, N4D, D8N, D30N, D61N, E64Q, N65D, Q108E, N1D/D61N, N1D/E64Q, N4D, D61N, N4D/E64Q, D8N/D61N, D8N/E64Q, D61N/E64Q, N1D/D30N, E64Q/Q108E, N1D/N4D/D8N, D61N/E64Q/N65Q, N1D/D61N/E64Q/Q108E, N4D/D61N, N4D/D61N/E64Q/Q108E, N1D/N65D, N1D/Q108E, N4D/D30N, D30N/Q108E, N65D/Q108E, D30N/Q180E, E64Q/N65D, D61N/E64Q/N65D, N1D/N4D/N65D, N71S/N72A/N77A, and N4D/D61N/N65D, preferably D30N/E64Q/N65D.

As mentioned herein the “/” refer to substitutions that are cumulative. Thus, by the mutation N4D/N65D, it is meant the following substitutions: N4D and N65D.

### IL-21 Variants

In a particular aspect, the immuno-stimulating cytokine is a variant of IL-21.

The IL-21 variant preferably has at least 80, 85, 90, 91, 92, 93, 94, 95, 96, 97, 98 or 99% of identity with the wildtype cytokine of SEQ ID NO: 89 or having 1 to 10 modifications selected from the group consisting of addition, deletion, substitution and combinations thereof with respect to the sequence of SEQ ID NO: 89.

Optionally, the IL-21 variant may comprise one of the following substitutions relative to human IL-21: R5A, R5D, R5E, R5G, R5H, R5I, R5K, R5L, R5M, R5N, R5Q, R5S, R5T, R5V, R5Y, I8A, I8D, I8E, I8G, I8N, I8S, R9A, R9D, R9E, R9G, R9H, R9I, R9K, R9L, R9M, R9N, R9Q, R9S, R9T, R9V, R9Y, R11D, R11S, Q12A, Q12D, Q12E, Q12N, Q12S, Q12T, Q12V, L13D, I14A, I14D, I14S, D15A, D15E, D15I, D15M, D15N, D15Q, D15S, D15T, D15V, I16D, I16E, Q19D, Y23D, R65D, R65G, R65P, I66D, I66G, I66P, N68Q, V69D, V69G, V69P, S70E, S70G, S70P, S70Y, S70T, K72D, K72G, K72P, K72A, K73A, K73D, K73E, K73G, K73H, K73I, K73N, K73P, K73Q, K73S, K73V, K75D, K75G, K75P, R76A, R76D, R76E, R76G, R76H, R76I, R76K, R76L, R76M, R76N, R76P, R76Q, R76S, R76T, R76V, R76Y, K77D, K77G, K77P, P78D, P79D, S80G, S80P, E109K, R110D, K112D, S113K, Q116A, Q116D, Q116E, Q116I, Q116K, Q116L, Q116M, Q116N, Q116S, Q116T, Q116V, K117D, I119A, I119D, I119E, I119M, I119N, I119Q, I119S, I119T, H120D and L123D, preferably R5E and R76E, R5E and R76A, R5A and R76A, R5Q and R76A, R5A and R76E, R5Q and R76E, R9E and R76E, R9A and R76E, R9E and R76A, R9A and R76A, D15N and S70T, D15N and I71L, D15N and K72A, D15N and K73A, S70T and K73Q, S70T and R76A, S70T and R76D, S70T and R76E, I71L and K73Q, I71L and R76A, I71L and R76D, I71L and R76E, K72A and K73Q, K72A and R76A, K72A and R76D, K72A and R76E, K73A and R76A, K73A and R76D, or K73A and R76E.

### Antigen Binding Domain Specifically Targeting Immune Cells

According to the invention, the antigen binding domain specifically binds to a target expressed on immune cells surface, particularly targets that are only or specifically expressed on immune cells. In particular, the antigen binding domain is not directed towards a target expressed on tumoral cells.

With regard to the “binding” capacity of the antigen binding domain, the terms “bind” or “binding” refer to antibodies including antibody fragments and derivatives that recognize and contact another peptide, polypeptide, protein or molecule. The terms “specific binding”, “specifically binds to,” “specific for,” “selectively binds” and “selective for” a particular target mean that the antigen binding domain recognizes and binds a specific target, but does not substantially recognize or bind other molecules in a sample. For example, an antibody that specifically (or preferentially) binds to an antigen is an antibody that binds the antigen for example with greater affinity, avidity, more readily, and/or with greater duration than it binds to other molecules. Preferably, the term “specific binding” means the contact between an antibody and an antigen with a binding affinity equal or lower than 10⁻⁷ M. In certain aspects, antibodies bind with affinities equal or lower than 10⁻⁸ M, 10⁻⁹ M or 10⁻¹⁰ M.

Optionally, the antigen-binding domain can be a Fab domain, a Fab′, a single-chain variable fragment (scFV) or a single domain antibody (sdAb). The antigen-binding domain preferably comprises a heavy chain variable region (VH) and a light chain variable region (VL).

When the antigen-binding domain is a Fab or a Fab′, the bifunctional molecule comprises one heavy chain and one light chain constant domain (i.e. CH and CL), the heavy chain being linked at its C-terminal end to the immuno-stimulating cytokine.

As used herein, the term “target” refers to a carbohydrate, lipid, peptide, polypeptide, protein, antigen or epitope that is specifically recognized or targeted by the antigen binding domain according to the invention and expressed on the external surface of immune cells. With regards to the expression of a target on the surface of immune cells, the term “expressed” refers to a target, such as carbohydrates, lipids, peptides, polypeptides, proteins, antigens or epitopes that are present or presented at the outer surface of a cell. The term “specifically expressed” mean that the target is expressed on immune cells, but is not substantially expressed by other cell type, particularly such as tumoral cells.

In one aspect, the target is specifically expressed by immune cells in a healthy subject or in a subject suffering from a disease, in particular such as a cancer. This means that the target has a higher expression level in immune cells than in other cells or that the ratio of immune cells expressing the target by the total immune cells is higher than the ratio of other cells expressing the target by the total other cells. Preferably the expression level or ratio is higher by a factor 2, 5, 10, 20, 50 or 100. More specifically, it can be determined for a particular type of immune cells, for instance T cells, more specifically CD8+ T cells, effector T cells or exhausted T cells, or in a particular context, for instance a subject suffering of a disease such as a cancer or an infection.

“Immune cells” as used herein refers to cells involved in innate and adaptive immunity for example such as white blood cells (leukocytes) which are derived from hematopoietic stem cells (HSC) produced in the bone marrow, lymphocytes (T cells, B cells, natural killer (NK) cells and Natural Killer T cells (NKT)) and myeloid-derived cells (neutrophil, eosinophil, basophil, monocyte, macrophage, dendritic cells). In particular, the immune cell can be selected in the non-exhaustive list comprising B cells, T cells, in particular CD4⁺ T cells and CD8⁺ T cells, NK cells, NKT cells, APC cells, macrophages, dendritic cells and monocytes.

Preferably, the antigen binding domain specifically binds to a target expressed immune cells selected from the group consisting of B-cells, T-cells, Natural killer, dendritic cells, monocytes and innate lymphoid cells (ILCs).

Even more preferably, the immune cell is a T cell. “T cell” or “T lymphocytes” as used herein includes for example CD4+ T cells, CD8+ T cells, T helper 1 type T cells, T helper 2 type T cells, T regulator, T helper 17 type T cells and inhibitory T cells. In a very particular aspect, the immune cell is an exhausted T cell.

In a particular aspect, the immune cell is an effector memory stem like T cell.

The target can be a receptor expressed at the surface of the immune cells, especially T cells. The receptor can be an inhibitor receptor. Alternatively, the receptor can be an activating receptor.

In one aspect, the target is selected from the group consisting PD-1, CD28, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CD3, PDL1; PDL2, and PDL1. Such targets are more particularly described in the Table E below.

TABLE E
Example of target of interest.

| Name | Official name | Uniprot reference |
|---|---|---|
| 2B4 | Natural killer cell receptor 2B4 (NK cell type I receptor protein 2B4, | Q07763 |
| | NKR2B4) (Non-MHC restricted killing associated) (SLAM family | |
| | member 4, SLAMF4) (Signaling lymphocytic activation molecule 4) | |
| | (CD antigen CD244) | |
| 4-1BB | Tumor necrosis factor receptor superfamily member 9 (4-1BB ligand | Q07011 |
| | receptor, CD137) | |
| BTLA | B- and T-lymphocyte attenuator (B- and T-lymphocyte-associated | Q7Z6A9 |
| | protein) (CD antigen CD272) | |
| CD101 | Immunoglobulin superfamily member 2, IgSF2 (Cell surface | Q93033 |
| | glycoprotein V7) (Glu-Trp-Ile EWI motif-containing protein 101, EWI- | |
| | 101) (CD antigen CD101) | |
| CD160 | CD160 antigen (Natural killer cell receptor BY55) | O95971 |
| CD27 | CD27 antigen (CD27L receptor) (T-cell activation antigen CD27) (T14) | P26842 |
| | (Tumor necrosis factor receptor superfamily member 7) (CD antigen | |
| | CD27) | |
| CD28 | T-cell-specific surface glycoprotein CD28 (TP44) | P10747 |
| CD28H | Transmembrane and immunoglobulin domain-containing protein 2 | Q96BF3 |
| | (CD28 homolog) (Immunoglobulin and proline-rich receptor 1, IGPR-1) | |
| CD3 | T-cell surface glycoprotein CD3 | P07766 (CD3e) |
| | | P04234 (CD3d) |
| | | P09693 (CD3g) |
| CD30 | Tumor necrosis factor ligand superfamily member 8 (CD30 ligand, | P32971 |
| | CD30-L) (CD antigen CD153) | |
| CD38 | ADP-ribosyl cyclase/cyclic ADP-ribose hydrolase 1 (ADPRC 1, cADPr | P28907 |
| | hydrolase 1) | |
| CD39 | Ectonucleoside triphosphate diphosphohydrolase-1 (NTPDase 1, | P49961 |
| | Ecto-apyrase, ATPDase 1, or Lymphoid cell activation antigen) | |
| CD40L | CD40 ligand (T-cell antigen Gp39, TNF-related activation protein, | P29965 |
| | Tumor necrosis factor ligand superfamily member 5, CD154) | |
| CD44 | CD44 antigen (Epican, Extracellular matrix receptor III, GP90 | P16070 |
| | lymphocyte homing/adhesion receptor, HUTCH-I, Heparan sulfate | |
| | proteoglycan, Hermes antigen, Hyaluronate receptor, Phagocytic | |
| | glycoprotein 1, Phagocytic glycoprotein I) | |
| CTLA-4 | Cytotoxic T-lymphocyte protein 4 (Cytotoxic T-lymphocyte-associated | P16410 |
| | antigen 4, CTLA-4) (CD antigen CD152) | |
| DR3 | Death receptor 3 (Tumor necrosis factor receptor superfamily | Q93038 |
| | member 25, WSL, Apo-3, LARD) | |
| GITR | Tumor necrosis factor receptor superfamily member 18 (Activation- | Q9Y5U5 |
| | inducible TNFR family receptor, Glucocorticoid-induced TNFR-related | |
| | protein, CD357) | |
| HVEM | Tumor necrosis factor receptor superfamily member 14 (Herpes virus | Q92956 |
| | entry mediator A, Herpesvirus entry mediator A, HveA) (Tumor | |
| | necrosis factor receptor-like 2, TR2) (CD antigen CD270) | |
| ICOS | Inducible T-cell costimulator (Activation-inducible lymphocyte | Q9Y6W8 |
| | immunomediatory molecule, CD278) | |
| LAG3 | Lymphocyte activation gene 3 protein, LAG-3 (Protein FDC) (CD | P18627 |
| | antigen CD223) | |
| LFA-1 | Leukocyte adhesion glycoprotein LFA-1 alpha chain (Integrin alpha-L, | P20701 |
| | CD11 antigen-like family member A) | |
| NKG2D | NKG2-D type II integral membrane protein (Killer cell lectin-like | P26718 |
| | receptor subfamily K member 1, NK cell receptor D, NKG2-D- | |
| | activating NK receptor, CD314) | |
| OX40 | Tumor necrosis factor receptor superfamily member 4 (ACT35 | P43489 |
| | antigen, AX transcriptionally-activated glycoprotein 1 receptor) | |
| PD-1 | Programmed cell death protein 1 (CD279) | Q15116 |
| PDL1 | Programmed cell death 1 ligand 1, PDL1, PD-L1, PDCD1 ligand 1, B7- | Q9NZQ7 |
| | H1, CD274 | |
| PDL2 | Programmed cell death 1 ligand 2, PD-1 ligand 2, PD-L2, PDCD1 ligand | Q9BQ51 |
| | 2, Programmed death ligand 2 (Butyrophilin B7-DC, B7-DC) (CD | |
| | antigen CD273) | |
| SIRPG | Signal-regulatory protein gamma, SIRP-gamma (CD172 antigen-like | Q9P1W8 |
| | family member B) (Signal-regulatory protein beta-2, SIRP-b2, SIRP- | |
| | beta-2) (CD antigen CD172g) | |
| TIGIT | T-cell immunoreceptor with Ig and ITIM domains (V-set and | Q495A1 |
| | immunoglobulin domain-containing protein 9) (V-set and | |
| | transmembrane domain-containing protein 3) | |
| Tim-1 | Hepatitis A virus cellular receptor 1 (T-cell immunoglobulin and | Q96D42 |
| | mucin domain-containing protein 1, Kidney injury molecule 1, KIM-1, | |
| | T-cell immunoglobulin mucin receptor 1, T-cell membrane protein 1, | |
| | CD365) | |
| TIM3 | Hepatitis A virus cellular receptor 2, HAVcr-2 (T-cell immunoglobulin | Q8TDQ0 |
| | and mucin domain-containing protein 3, TIMD-3) (T-cell | |
| | immunoglobulin mucin receptor 3, TIM-3) (T-cell membrane protein | |
| | 3) | |

Then, in this aspect, the antigen binding domain specifically binds a target selected from the group consisting PD-1, CD28, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CD3, PDL2 and PDL1.

In a particular aspect, the immune cell is an exhausted T cell or an effector memory stem like T cell and the target of the antigen binding domain is a factor expressed on the surface of exhausted T cells or effector memory stem like T cells. T cell exhaustion is a state of T cell progressive loss of function, proliferation capacity and cytotoxic potential, eventually leading to their deletion. T cell exhaustion can be triggered by several factors such as persistent antigen exposure or inhibitory receptors including PD-1, TIM3, CD244, CTLA-4, LAG-3, BTLA, TIGIT and CD160. Preferably, such factor, in particular such exhaustion factor, is selected from the group consisting of PD-1, TIM3, CD244, CTLA-4, LAG3, BTLA, TIGIT and CD160.

In a preferred aspect, the antigen binding domain has an antagonist activity on the target.

Numerous antibodies directed against PD-1, TIM3, CD244, CTLA-4, LAG-3, BTLA, TIGIT and CD160 have already been described in the art.

Several anti-PD-1 are already clinically approved, and others are still in clinical developments. For instance, the anti-PD1 antibody can be selected from the group consisting of Pembrolizumab (also known as Keytruda lambrolizumab, MK-3475), Nivolumab (Opdivo, MDX-1106, BMS-936558, ONO-4538), Pidilizumab (CT-011), Cemiplimab (Libtayo), Camrelizumab, AUNP12, AMP-224, AGEN-2034, BGB-A317 (Tisleizumab), PDR001 (spartalizumab), MK-3477, SCH-900475, PF-06801591, JNJ-63723283, genolimzumab (CBT-501), LZM-009, BCD-100, SHR-1201, BAT-1306, AK-103 (HX-008), MEDI-0680 (also known as AMP-514) MEDI0608, JS001 (see Si-Yang Liu et al., J. Hematol. Oncol.10:136 (2017)), BI-754091, CBT-501, INCSHR1210 (also known as SHR-1210), TSR-042 (also known as ANB011), GLS-010 (also known as WBP3055), AM-0001 (Armo), STI-1110 (see WO 2014/194302), AGEN2034 (see WO 2017/040790), MGA012 (see WO 2017/19846), or IBI308 (see WO 2017/024465, WO 2017/025016, WO 2017/132825, and WO 2017/133540), monoclonal antibodies 5C4, 17D8, 2D3, 4H1, 4A11, 7D3, and 5F4, described in WO 2006/121168. Bifunctional or bispecific molecules targeting PD-1 are also known such as RG7769 (Roche), XmAb20717 (Xencor), MEDI5752 (AstraZeneca), FS118 (F-star), SL-279252 (Takeda) and XmAb23104 (Xencor).

In a particular aspect, the anti-PD1 antibody can be Pembrolizumab (also known as Keytruda lambrolizumab, MK-3475) or Nivolumab (Opdivo, MDX-1106, BMS-936558, ONO-4538).

Antibodies directed against TIM3 and bifunctional or bispecific molecules targeting TIM3 are also known such as Sym023, TSR-022, MBG453, LY3321367, INCAGN02390, BGTB-A425, LY3321367, RG7769 (Roche). In some aspects, a TFM-3 antibody is as disclosed in International Patent Application Publication Nos. WO2013006490, WO2016/161270, WO 2018/085469, or WO 2018/129553, WO 2011/155607, U.S. Pat. No. 8,552,156, EP 2581113 and U.S 2014/044728.

Antibodies directed against CTLA-4 and bifunctional or bispecific molecules targeting CTLA-4 are also known such as ipilimumab, tremelimumab, MK-1308, AGEN-1884, XmAb20717 (Xencor), MEDI5752 (AstraZeneca). Anti-CTLA-4 antibodies are also disclosed in WO18025178, WO19179388, WO19179391, WO19174603, WO19148444, WO19120232, WO19056281, WO19023482, WO18209701, WO18165895, WO18160536, WO18156250, WO18106862, WO18106864, WO18068182, WO18035710, WO18025178, WO17194265, WO17106372, WO17084078, WO17087588, WO16196237, WO16130898, WO16015675, WO12120125, WO09100140 and WO07008463.

Antibodies directed against LAG3 and bifunctional or bispecific molecules targeting LAG-3 are also known such as BMS-986016, IMP701, MGD012 or MGD013 (bispecific PD-1 and LAG-3 antibody). Anti-LAG-3 antibodies are also disclosed in WO2008132601, EP2320940, WO19152574.

Antibodies directed against BTLA are also known in the art such as hu Mab8D5, hu Mab8A3, hu Mab21H6, hu Mab19A7, or hu Mab4C7. The antibody TAB004 against BTLA are currently under clinical trial in subjects with advanced malignancies. Anti-BTLA antibodies are also disclosed in WO08076560, WO10106051 (e.g., BTLA8.2), WO11014438 (e.g., 4C7), WO17096017 and WO17144668 (e.g., 629.3).

Antibodies directed against TIGIT are also known in the art, such as BMS-986207 or AB154, BMS-986207 CPA.9.086, CHA.9.547.18, CPA.9.018, CPA.9.027, CPA.9.049, CPA.9.057, CPA.9.059, CPA.9.083, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8 as disclosed in WO19232484. Anti-TIGIT antibodies are also disclosed in WO16028656, WO16106302, WO16191643, WO17030823, WO17037707, WO17053748, WO17152088, WO18033798, WO18102536, WO18102746, WO18160704, WO18200430, WO18204363, WO19023504, WO19062832, WO19129221, WO19129261, WO19137548, WO19152574, WO19154415, WO19168382 and WO19215728.

Antibodies directed against CD160 are also known in the art, such as CL1-R2 CNCM I-3204 as disclosed in WO06015886, or others as disclosed in WO10006071, WO10084158, WO18077926.

Antibodies directed against PD-L1 are also known in the art. Examples of monoclonal antibodies that bind to human PD-L1, and useful for the present invention, are described in WO 2007/005874, WO 2010/036959, WO 2010/077634, WO 2010/089411, WO 2013/019906, WO 2013/079174, WO 2014/100079, WO 2015/061668, and U.S. Pat. Nos. 8,552,154, 8,779,108 and 8,383,796. Specific anti-human PD-L1 monoclonal antibodies include, for example without limitation, avelumab (MSB0010718C), durvalumab (MEDI4736, an engineered IgG1 kappa monoclonal antibody with triple mutations in the Fc domain to remove ADCC), atezolizumab (MPLDL3280A), MPDL3280A (an IgG1-engineered anti-PD-L1 antibody), and BMS-936559 (a fully human, anti-PD-L1, IgG4 monoclonal antibody).

In a preferred aspect, the antigen binding domain of the bifunctional molecule is an antibody, a fragment or a derivative thereof that is specific to PD-1, CTLA-4, BTLA, TIGIT, LAG3 and TIM3.

In another particular aspect, the target is PD-1 and the antigen binding domain of the bifunctional molecule is an antibody, a fragment or a derivative thereof or an antibody mimic that is specific to PD-1. Then, in a particular aspect, the antigen binding domain comprised in the bifunctional molecule according to the invention is an anti-PD1 antibody or antigen binding fragment thereof, preferably a human, humanized or chimeric anti-PD1 antibody or antigen binding fragment thereof. Preferably, the antigen binding domain is an antagonist of PD-1.

In another particular aspect, the target is CTLA-4 and the antigen binding domain of the bifunctional molecule is an antibody, a fragment or a derivative thereof or an antibody mimic that is specific to CTLA-4. Then, in a particular aspect, the antigen binding domain comprised in the bifunctional molecule according to the invention is an anti-CTLA-4 antibody or antigen binding fragment thereof, preferably a human, humanized or chimeric anti-CTLA-4 antibody or antigen binding fragment thereof. Preferably, the antigen binding domain is an antagonist of CTLA-4.

In another particular aspect, the target is BTLA and the antigen binding domain of the bifunctional molecule is an antibody, a fragment or a derivative thereof or an antibody mimic that is specific to BTLA. Then, in a particular aspect, the antigen binding domain comprised in the bifunctional molecule according to the invention is an anti-BTLA antibody or antigen binding fragment thereof, preferably a human, humanized or chimeric anti-BTLA antibody or antigen binding fragment thereof. Preferably, the antigen binding domain is an antagonist of BTLA.

In another particular aspect, the target is TIGIT and the antigen binding domain of the bifunctional molecule is an antibody, a fragment or a derivative thereof or an antibody mimic that is specific to TIGIT. Then, in a particular aspect, the antigen binding domain comprised in the bifunctional molecule according to the invention is an anti-TIGIT antibody or antigen binding fragment thereof, preferably a human, humanized or chimeric anti-TIGIT antibody or antigen binding fragment thereof. Preferably, the antigen binding domain is an antagonist of TIGIT.

In another particular aspect, the target is LAG-3 and the antigen binding domain of the bifunctional molecule is an antibody, a fragment or a derivative thereof or an antibody mimic that is specific to LAG-3. Then, in a particular aspect, the antigen binding domain comprised in the bifunctional molecule according to the invention is an anti-LAG-3 antibody or antigen binding fragment thereof, preferably a human, humanized or chimeric anti-LAG-3 antibody or antigen binding fragment thereof. Preferably, the antigen binding domain is an antagonist of LAG-3.

In another particular aspect, the target is TIM3 and the antigen binding domain of the bifunctional molecule is an antibody, a fragment or a derivative thereof or an antibody mimic that is specific to TIM3. Then, in a particular aspect, the antigen binding domain comprised in the bifunctional molecule according to the invention is an anti-TIM3 antibody or antigen binding fragment thereof, preferably a human, humanized or chimeric anti-TIM3 antibody or antigen binding fragment thereof. Preferably, the antigen binding domain is an antagonist of TIM3.

In a very specific aspect of the present disclosure, the antigen binding domain targets PD-1 and is derived from the antibody disclosed in WO2020/127366, the disclosure thereof being incorporated herein by reference.

Then, the antigen-binding domain comprises:
(i) a heavy chain variable domain comprising HCDR1, HCDR2 and HCDR3, and
(ii) a light chain variable domain comprising LCDR1, LCDR2 and LCDR3, wherein:
   the heavy chain CDR1 (HCDR1) comprises or consists of an amino acid sequence of SEQ ID NO: 51, optionally with one, two or three modification(s) selected from substitution(s), addition(s), deletion(s) and any combination thereof at any position but position 3 of SEQ ID NO: 51;
   the heavy chain CDR2 (HCDR2) comprises or consists of an amino acid sequence of SEQ ID NO: 53, optionally with one, two or three modification(s) selected from substitution(s), addition(s), deletion(s) and any combination thereof at any position but positions 13, 14 and 16 of SEQ ID NO: 53;
   the heavy chain CDR3 (HCDR3) comprises or consists of an amino acid sequence of SEQ ID NO: 54 wherein X1 is D or E and X2 is selected from the group consisting of T, H, A, Y, N, E and S, preferably in the group consisting of H, A, Y, N, E; optionally with one, two or three modification(s) selected from substitution(s), addition(s), deletion(s) and any combination thereof at any position but positions 2, 3, 7 and 8 of SEQ ID NO: 54;
   the light chain CDR1 (LCDR1) comprises or consists of an amino acid sequence of SEQ ID NO: 63 wherein X is G or T, optionally with one, two or three modification(s) selected from substitution(s), addition(s), deletion(s) and any combination thereof at any position but positions 5, 6, 10, 11 and 16 of SEQ ID NO: 63;
   the light chain CDR2 (LCDR2) comprises or consists of an amino acid sequence of SEQ ID NO: 66, optionally with one, two or three modification(s) selected from substitution(s), addition(s), deletion(s) and any combination thereof; and
   the light chain CDR3 (LCDR3) comprises or consists of an amino acid sequence of SEQ ID NO: 16, optionally with one, two or three modification(s) selected from substitution(s), addition(s), deletion(s) and any combination thereof at any position but positions 1, 4 and 6 of SEQ ID NO: 16.

In one aspect, the antigen-binding domain comprises:
(i) a heavy chain variable domain comprising HCDR1, HCDR2 and HCDR3, and
(ii) a light chain variable domain comprising LCDR1, LCDR2 and LCDR3, wherein:
   the heavy chain CDR1 (HCDR1) comprises or consists of an amino acid sequence of SEQ ID NO: 51, optionally with one, two or three modification(s) selected from substitution(s), addition(s), deletion(s) and any combination thereof at any position but position 3 of SEQ ID NO: 51;
   the heavy chain CDR2 (HCDR2) comprises or consists of an amino acid sequence of SEQ ID NO: 53, optionally with one, two or three modification(s) selected from substitution(s), addition(s), deletion(s) and any combination thereof at any position but positions 13, 14 and 16 of SEQ ID NO: 53;
   the heavy chain CDR3 (HCDR3) comprises or consists of an amino acid sequence of SEQ ID NO: 54 wherein either X1 is D and X2 is selected from the group consisting of T, H, A, Y, N, E, and S preferably in the group consisting of H, A, Y, N, E; or X1 is E and X2 is selected from the group consisting of T, H, A, Y, N, E and S, preferably in the group consisting of H, A, Y, N, E and S; optionally with one, two or three modification(s) selected from substitution(s), addition(s), deletion(s) and any combination thereof at any position but positions 2, 3, 7 and 8 of SEQ ID NO: 54;
   the light chain CDR1 (LCDR1) comprises or consists of an amino acid sequence of SEQ ID NO: 63 wherein X is G or T, optionally with one, two or three modification(s) selected from substitution(s), addition(s), deletion(s) and any combination thereof at any position but positions 5, 6, 10, 11 and 16 of SEQ ID NO: 63;
   the light chain CDR2 (LCDR2) comprises or consists of an amino acid sequence of SEQ ID NO: 66, optionally with one, two or three modification(s) selected from substitution(s), addition(s), deletion(s) and any combination thereof; and
   the light chain CDR3 (LCDR3) comprises or consists of an amino acid sequence of SEQ ID NO: 16, optionally with one, two or three modification(s) selected from substitution(s), addition(s), deletion(s) and any combination thereof at any position but positions 1, 4 and 6 of SEQ ID NO: 16.

In another embodiment, the antigen-binding domain comprises or consists essentially of: (i) a heavy chain comprising a CDR1 of SEQ ID NO: 51, a CDR2 of SEQ ID NO: 53 and a CDR3 of SEQ ID NO: 55, 56, 57, 58, 59, 60, 61 or 62; and (ii) a light chain comprising a CDR1 of SEQ ID NO: 64 or SEQ ID NO: 65, a CDR2 of SEQ ID NO: 66and a CDR3 of SEQ ID NO: 16.

In another aspect, the antigen-binding domain comprises or consists essentially of:
(i) a heavy chain comprising a CDR1 of SEQ ID NO: 51, a CDR2 of SEQ ID NO: 53 and a CDR3 of SEQ ID NO: 55; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 56; and (ii) a light chain comprising a CDR1 of SEQ ID NO: 64, a CDR2 of SEQ ID NO: 66 and a CDR3 of SEQ ID NO: 16, or
(i) a heavy chain comprising a CDR1 of SEQ ID NO: 51, a CDR2 of SEQ ID NO: 53 and a CDR3 of SEQ ID NO: 57; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 58; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 59; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 60; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 61; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 62; and (ii) a light chain comprising a CDR1 of SEQ ID NO: 64, a CDR2 of SEQ ID NO: 66 and a CDR3 of SEQ ID NO: 16, or
(i) a heavy chain comprising a CDR1 of SEQ ID NO: 51, a CDR2 of SEQ ID NO: 53 and a CDR3 of SEQ ID NO: 55; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 56; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 57; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 58; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 59; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 60; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 61; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 62; and (ii) a light chain comprising a CDR1 of SEQ ID NO: 65, a CDR2 of SEQ ID NO: 66 and a CDR3 of SEQ ID NO: 16.

In an aspect, the antigen-binding domain comprises or consists essentially of:
(a) a heavy chain variable region (VH) comprising or consisting of an amino acid sequence of SEQ ID NO: 17, wherein X1 is D or E and X2 is selected from the group consisting of T, H, A, Y, N, E and S preferably in the group consisting of H, A, Y, N, E; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;
(b) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26.

In another aspect, the antigen-binding domain comprises or consists essentially of:
(a) a heavy chain variable region (VH) comprising or consisting of an amino acid sequence of SEQ ID NO:18, 19, 20, 21, 22, 23, 24 or 25, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 18, 19, 20, 21, 22, 23, 24 or 25 respectively;
(b) a light chain variable region (VL) comprising or consisting of an amino acid sequence of SEQ ID NO: 27 or SEQ ID NO: 28, optionally with one, two or three modification(s) selected from substitution(s), addition(s), deletion(s) and any combination thereof at any position positions 3, 4, 7, 14, 17, 18, 28, 29, 33, 34, 39, 42, 44, 50, 81, 88, 94, 97, 99 and 105 of SEQ ID NO: 27 or SEQ ID NO: 28.

In another aspect, the antigen-binding domain comprises or consists essentially of:
(a) a heavy chain variable region (VH) comprising or consisting of an amino acid sequence of SEQ ID NO: 18, 19, 20, 21, 22, 23, 24 or 25;
(b) a light chain variable region (VL) comprising or consisting of an amino acid sequence of SEQ ID NO: 27 or SEQ ID NO: 28.

In one aspect, the bifunctional molecule comprises framework regions, in particular heavy chain variable region framework regions (HFR) HFR1, HFR2, HFR3 and HFR4 and light chain variable region framework regions (LFR) LFR1, LFR2, LFR3 and LFR4, especially HFR1, HFR2, HFR3 and HFR4 comprising an amino acid sequence of SEQ ID NOs: 41, 42, 43 and 44, respectively, optionally with one, two or three modification(s) selected from substitution(s), addition(s), deletion(s) and any combination thereof at any position but positions 27, 29 and 32 of HFR3, i.e., of SEQ ID NO: 43. Preferably, the bifunctional molecule comprises HFR1 of SEQ ID NO: 41, HFR2 of SEQ ID NO: 42, HFR3 of SEQ ID NO: 43 and HFR4 of SEQ ID NO: 44. In addition, the bifunctional molecule may comprise light chain variable region framework regions (LFR) LFR1, LFR2, LFR3 and LFR4 comprising an amino acid sequence of SEQ ID NOs: 45, 46, 47 and 48, respectively, optionally with one, two or three modification(s) selected from substitution(s), addition(s), deletion(s) and any combination thereof. Preferably, the bifunctional molecule comprises LFR1 of SEQ ID NO: 45, LFR2 of SEQ ID NO: 46, LFR3 of SEQ ID NO: 47 and LFR4 of SEQ ID NO: 48.

In another aspect, the antigen-binding domain comprises or consists essentially of any of the following combinations of a heavy chain variable region (VH) and a light chain variable region (VL):

| VH (SEQ ID NO:), optionally with one, two or | |
|---|---|
| three modification(s) selected from | VL (SEQ ID NO:), optionally with one, two or |
| substitution(s), addition(s), deletion(s) and any | three modification(s) selected from |
| combination thereof at any position but | substitution(s), addition(s), deletion(s) and any |
| positions 7, 16, 17, 20, 33, 38, 43, 46, 62, 63, 65, | combination thereof at any position positions 3, |
| 69, 73, 76, 78, 80, 84, 85, 88, 93, 95, 96, 97, 98, | 4, 7, 14, 17, 18, 28, 29, 33, 34, 39, 42, 44, 50, 81, |
| 100, 101, 105, 106 and 112 of SEQ ID NO: | 88, 94, 97, 99 and 105 of SEQ ID NO: |
| 18 | 27 |
| 18 | 28 |
| 19 | 27 |
| 19 | 28 |
| 20 | 27 |
| 20 | 28 |
| 21 | 27 |
| 21 | 28 |
| 22 | 27 |
| 22 | 28 |
| 23 | 27 |
| 23 | 28 |
| 24 | 27 |
| 24 | 28 |
| 25 | 27 |
| 25 | 28 |

In very particular aspect, the antigen-binding domain comprises or consists essentially of a heavy chain variable region (VH) of SEQ ID NO: 24 and a light chain variable region (VL) of SEQ ID NO: 28.

### Preferred Combinations

Particular combinations of targets specifically expressed on immune cells surface and of immuno-stimulating cytokines are contemplated herein.

In a first aspect, the bifunctional molecule comprises an antigen binding domain that binds (and preferably antagonizes) PD-1 and an immuno-stimulating cytokine selected from the group consisting of IL-2, IL-12, IL-15, IL-21 and variants thereof, and IL-7. Optionally, the bifunctional molecule comprises an antigen binding domain that binds (and preferably antagonizes) PD-1 and an immuno-stimulating cytokine selected from the group consisting of IL-12, IL-15, IL-21 and variants thereof, and IL-7. Optionally, the bifunctional molecule comprises one of the following combinations: a) an antigen binding domain that binds PD-1 and IL-2 or a variant thereof; b) an antigen binding domain that binds PD-1 and IL-7; c) an antigen binding domain that binds PD-1 and IL-12 or a variant thereof; d) an antigen binding domain that binds PD-1 and IL-15 or a variant thereof; e) an antigen binding domain that binds PD-1 and IL-15 or a variant thereof; or f) an antigen binding domain that binds PD-1 and IL-21 or a variant thereof.

In a second aspect, the bifunctional molecule comprises an antigen binding domain that binds (and preferably antagonizes) PD-L1 and an immuno-stimulating cytokine selected from the group consisting of IL-2, IL-12, IL-15, IL-21 and variants thereof, and IL-7. Optionally, the bifunctional molecule comprises one of the following combinations: a) an antigen binding domain that binds PD-L1 and IL-2 or a variant thereof; b) an antigen binding domain that binds PD-L1 and IL-7; c) an antigen binding domain that binds PD-L1 and IL-12 or a variant thereof; d) an antigen binding domain that binds PD-L1 and IL-15 or a variant thereof; e) an antigen binding domain that binds PD-1 and IL-15 or a variant thereof; or f) an antigen binding domain that binds PD-L1 and IL-21 or a variant thereof.

In a third aspect, the bifunctional molecule comprises an antigen binding domain that binds (and preferably antagonizes) PD-L2 and an immuno-stimulating cytokine selected from the group consisting of IL-2, IL-12, IL-15, IL-21 and variants thereof, and IL-7. Optionally, the bifunctional molecule comprises one of the following combinations: a) an antigen binding domain that binds PD-L2 and IL-2 or a variant thereof; b) an antigen binding domain that binds PD-L2 and IL-7; c) an antigen binding domain that binds PD-L2 and IL-12 or a variant thereof; d) an antigen binding domain that binds PD-L2 and IL-15 or a variant thereof; e) an antigen binding domain that binds PD-L2 and IL-15 or a variant thereof; or f) an antigen binding domain that binds PD-L2 and IL-21 or a variant thereof.

In a fourth aspect, the bifunctional molecule comprises an antigen binding domain that binds (and preferably antagonizes) CTLA-4 and an immuno-stimulating cytokine selected from the group consisting of IL-2, IL-12, IL-15, IL-21 and variants thereof, and IL-7. Optionally, the bifunctional molecule comprises one of the following combinations: a) an antigen binding domain that binds CTLA-4 and IL-2 or a variant thereof; b) an antigen binding domain that binds CTLA-4 and IL-7; c) an antigen binding domain that binds CTLA-4 and IL-12 or a variant thereof; d) an antigen binding domain that binds CTLA-4 and IL-15 or a variant thereof; e) an antigen binding domain that binds CTLA-4 and IL-15 or a variant thereof; or f) an antigen binding domain that binds CTLA-4 and IL-21 or a variant thereof.

In a fifth aspect, the bifunctional molecule comprises an antigen binding domain that binds (and preferably antagonizes) TIM3 and an immuno-stimulating cytokine selected from the group consisting of IL-2, IL-12, IL-15, IL-21 and variants thereof, and IL-7. Optionally, the bifunctional molecule comprises one of the following combinations: a) an antigen binding domain that binds TIM3 and IL-2 or a variant thereof; b) an antigen binding domain that binds TIM3 and IL-7; c) an antigen binding domain that binds TIM3 and IL-12 or a variant thereof; d) an antigen binding domain that binds TIM3 and IL-15 or a variant thereof; e) an antigen binding domain that binds PD-1 and IL-15 or a variant thereof; or f) an antigen binding domain that binds TIM3 and IL-21 or a variant thereof.

### Peptide Linker

In a particular aspect, the bifunctional molecule according to the invention further comprises a peptide linker connecting the antigen binding domain and the immuno-stimulating cytokine to the Fc chain. The peptide linker usually has a length and flexibility enough to ensure that the immuno-stimulating cytokine and the antigen binding domain connected with the linker in between have enough freedom in space to exert their functions.

In an aspect of the disclosure, the immuno-stimulating cytokine is preferably linked to the Fc chain through a peptide linker. In an aspect of the disclosure, the antigen binding domain can be linked to the Fc chain by the hinge naturally found in a heavy chain for connecting VH domain, especially CH1 domain to the CH2 domain of the Fc chain.

As used herein, the term “linker” refers to a sequence of at least one amino acid. Such a linker may be useful to prevent steric hindrances. The linker is usually 3-44 amino acid residues in length. Preferably, the linker has 3-30 amino acid residues. In some aspects, the linker has 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues.

The linker sequence may be a naturally occurring sequence or a non-naturally occurring sequence. If used for therapeutic purposes, the linker is preferably non-immunogenic in the subject to which the bifunctional molecule is administered. One useful group of linker sequences are linkers derived from the hinge region of heavy chain antibodies as described in WO 96/34103 and WO 94/04678. Other examples are poly-alanine linker sequences. Further preferred examples of linker sequences are Gly/Ser linkers of different length including (Gly4Ser)4, (Gly4Ser)3, (Gly4Ser)2, Gly4Ser, Gly3Ser, Gly3, Gly2ser and (Gly3Ser2)3, in particular (Gly4Ser)3. Preferably, the linker is selected from the group consisting of (Gly4Ser)4, (Gly4Ser)3, and (Gly3Ser2)3. Even more preferably, the linker is (GGGGS)3.

In one embodiment, the linker comprised in the bifunctional molecule is selected in the group consisting of (Gly4Ser)4, (Gly4Ser)3, (Gly4Ser)2, Gly4Ser, Gly3Ser, Gly3, Gly2ser and (Gly3Ser2)3, preferably is (Gly4Ser)3. Preferably, the linker is selected from the group consisting of (Gly4Ser)4, (Gly4Ser)3, and (Gly3Ser2)3.

In a particular embodiment, the linker the linker comprised in the bifunctional molecule is selected in the group consisting of linker having a sequence as set forth in SEQ ID NO: 67, 68, 69 or 70.

### Fc Domain

The Fc domain of the bifunctional molecule can be part of the antigen binding moiety, especially a heavy chain of an IgG immunoglobulin. Indeed, when the antigen binding domain is a Fab, the bifunctional molecule may comprise one heavy chain, including the variable heavy chain (VH), CH1, hinge, CH2 and CH3 domains. However, the bifunctional molecule may also have other structures such as scFv, or diabody. For instance, it may comprise an Fc domain linked to antibody derivative such as.

The Fc domain can be derived from a heavy chain constant domain of a human immunoglobulin heavy chain, for example, IgG1, IgG2, IgG3, IgG4, or other classes. Preferably, the bifunctional molecule comprises an IgG1 or an IgG4 heavy chain constant domain.

Preferably, the Fc domain comprises CH2 and CH3 domains. Optionally, it can include all or a portion of the hinge region, the CH2 domain and/or the CH3 domain. In some aspects, the CH2 and/or a CH3 domains are derived from a human IgG4 or IgG1 heavy chain. Preferably, the Fc domain includes all or a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4. More preferably, the hinge region is derived from a human or humanized IgG1 or IgG4 heavy chain.

The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG1. In some aspects, the first cysteine within the human IgG1 hinge region is mutated to another amino acid, preferably serine.

The hinge region of IgG4 is known to form interchain disulfide bonds inefficiently. However, a suitable hinge region for the present invention can be derived from the IgG4 hinge region, preferably containing a mutation that enhances correct formation of disulfide bonds between heavy chain-derived moieties (Angal S, et al. (1993) Mol. Immunol., 30:105-8). More preferably, the hinge region is derived from a human IgG4 heavy chain.

The bifunctional molecule comprises a dimeric Fc domain. Accordingly, two monomers comprise each one a Fc chain, the Fc chains being able to form a dimeric Fc domain. The dimeric Fc domain can be a homodimer, each Fc monomer being identical or essentially identical. Alternatively, the dimeric Fc domain can be a heterodimer, each Fc monomer being different and complementary in order to promote the formation of the heterodimeric Fc domain.

More specifically, the Fc domain is a heterodimeric Fc domain. Heterodimeric Fc domains are made by altering the amino acid sequence of each monomer. The heterodimeric Fc domains rely on amino acid variants in the constant regions that are different on each chain to promote heterodimeric formation and/or allow for ease of purification of heterodimers over the homodimers. There are a number of mechanisms that can be used to generate the heterodimers of the present invention. In addition, as will be appreciated by those in the art, these mechanisms can be combined to ensure high heterodimerization. Thus, amino acid variants that lead to the production of heterodimers are referred to as “heterodimerization variants”. Heterodimerization variants can include steric variants (e.g. the “knobs and holes” or “skew” variants described below and the “charge pairs” variants described below) as well as “pi variants”, which allows purification of homodimers away from heterodimers. WO2014/145806, hereby incorporated by reference in its entirety, discloses useful mechanisms for heterodimerization include “knobs and holes”, “electrostatic steering” or “charge pairs”, pi variants, and general additional Fc variants. See also, Ridgway et al., Protein Engineering 9(7):617 (1996); Atwell et al., J. Mol. Biol. 1997 270:26; U.S. Pat. No. 8,216,805, Merchant et al., Nature Biotech. 16:677 (1998), all of which are hereby incorporated by reference in their entirety. For “electrostatic steering” see Gunasekaran et al., J. Biol. Chem. 285(25): 19637 (2010), hereby incorporated by reference in its entirety. For pi variants, see US 2012/0149876 hereby incorporated by reference in its entirety.

Then, in a preferred aspect, the heterodimeric Fc domain comprises a first Fc chain and a complementary second Fc chain based on the “knobs and holes” technology. For instance, the first Fc chain is a “knob” or K chain, meaning that it comprises the substitution characterizing a knob chain, and the second Fc chain is a “hole” or H chain, meaning that it comprises the substitution characterizing a hole chain. And vice versa, the first Fc chain is a “hole” or H chain, meaning that it comprises the substitution characterizing a hole chain, and the second Fc chain is a “knob” or K chain, meaning that it comprises the substitution characterizing a knob chain. In a preferred aspect, the first Fc chain is a “hole” or H chain and the second Fc chain is a “knob” or K chain.

Optionally, the heterodimeric Fc domain may comprise one heterodimeric Fc chain which comprises the substitutions as shown in the following table F and the other heterodimeric Fc chain comprising the substitutions as shown in the following table F.

TABLE F
(the numbering being according to EU index)

| Fc chain having the following substitutions | The complementary Fc chain having the |
|---|---|
| (Hole chain or H chain) | following substitutions (Knob chain or K chain) |
| D221E/P228E/L368E | D221R/P228R/K409R |
| C220E/P228E/368E | C220R/E224R/P228R/K409R |
| S364K/E357Q | L368D/K370S |
| L368D/K370S | S364K |
| L368E/K370S | S364K |
| T411T/E360E/Q362E | D401K |
| L368D/K370S | S364K/E357L |
| K370S | S364K/E357Q |
| T366S/L368A/Y407V | T366W |
| T366S/L368A/Y407V/Y349C | T366W/S354C |
| F368D/K370S | S364K |
| F368D/K370S | S364K/E357F |
| F368D/K370S | S364K/E357Q |
| T411E/K360E/Q362E | D401K |
| F368E/K370S | S364K |
| K370S | S364K/E357Q |
| T366S/F368A/Y407V | T366W |
| T366S/L368A/Y407V/Y349C | T366W/S354C |

In a preferred aspect, the first Fc chain is a “hole” or H chain and comprises the substitutions T366S/L368A/Y407V/Y349C and the second Fc chain is a “knob” or K chain and comprises the substitutions T366W/S354C.

Optionally, the Fc chain may further comprise additional substitutions.

In particular, for bifunctional molecules that target cell-surface molecules, especially those on immune cells, abrogating effector functions may be required. Engineering Fc regions may also be desired to either reduce or increase the effector function of the bifunctional molecules.

In certain aspects, amino acid modifications may be introduced into the Fc region to generate an Fc region variant. In certain aspects, the Fc region variant possesses some, but not all, effector functions. Such bifunctional molecules may be useful, for example, in applications in which the half-life of the antibody in vivo is important, yet certain effector functions are unnecessary or deleterious. Numerous substitutions or substitutions or deletions with altered effector function are known in the art.

In one aspect, the constant region of the Fc domain contains a mutation that reduces affinity for an Fc receptor or reduces Fc effector function. For example, the constant region can contain a mutation that eliminates the glycosylation site within the constant region of an IgG heavy chain. Preferably, the CH2 domain contains a mutation that eliminates the glycosylation site within the CH2 domain.

In a particular aspect, the Fc domain is modified to increase the binding to FcRn, thereby increasing the half-life of the bifunctional molecule. In another aspect or additional aspect, the Fc domain is modified to decrease the binding to FcγR, thereby reducing ADCC or CDC, or to increase the binding to FcγR, thereby increasing ADCC or CDC.

The alteration of amino acids near the junction of the Fc portion and the non-Fc portion can dramatically increase the serum half-life of the Fc fusion protein as shown in WO 01/58957. Accordingly, the junction region of a protein or polypeptide of the present invention can contain alterations that, relative to the naturally-occurring sequences of an immunoglobulin heavy chain and erythropoietin, preferably lie within about 10 amino acids of the junction point. These amino acid changes can cause an increase in hydrophobicity. In one embodiment, the constant region is derived from an IgG sequence in which the C-terminal lysine residue is replaced. Preferably, the C-terminal lysine of an IgG sequence is replaced with a non-lysine amino acid, such as alanine or leucine, to further increase serum half-life.

In one embodiment, the constant region of the Fc domain has one of the mutations described in the Table G below, or any combination thereof.

TABLE G
Suitable human engineered Fc domain of an antibody.

| Engineered Fc | Isotype | Mutations | FcR/C1q Binding | Effector Function |
|---|---|---|---|---|
| hIgG1e1-Fc | IgG1 | T250Q/M428L | Increased | Increased half-life |
| | | | binding to FcRn | |
| hIgG1e2-Fc | IgG1 | M252Y/S254T/T256E + | Increased | Increased half-life |
| | | H433K/N434F | binding to FcRn | |
| hIgG1e3-Fc | IgG1 | E233P/L234V/L235A/G236A + | Reduced binding | Reduced ADCC and |
| | | A327G/A330S/P331S | to FcγRI | CDC |
| hIgG1e4-Fc | IgG1 | E333A | Increased | Increased ADCC and |
| | | | binding to | CDC |
| | | | FcγRIIIa | |
| hIgG1e5-Fc | IgG1 | S239D/A330L/I332E | Increased | Increased ADCC |
| | | | binding to | |
| | | | FcγRIIIa | |
| hIgG1e6-Fc | IgG1 | P257I/Q311 | Increased | Unchanged half-life |
| | | | binding to FcRn | |
| hIgG1e7-Fc | IgG1 | K326W/E333S | Increased | Increased CDC |
| | | | binding to C1q | |
| hIgG1e9-Fc | IgG1 | S239D/I332E/G236A | Increased | Increased |
| | | | FcγRIIa/FcγRIIb | macrophage |
| | | | ratio | phagocytosis |
| hIgG1e9-Fc | IgG1 | N297A | Reduced binding | Reduced ADCC and |
| | | | to FcγRI | CDC |
| hIgG1e9-Fc | IgG1 | LALA (L234A/L235A) | Reduced binding | Reduced ADCC and |
| | | | to FcγRI | CDC |
| hIgG1e10- | IgG1 | N297A + YTE | Reduced binding | Reduced ADCC and |
| Fc | | (N298A + | to FcγRI | CDC |
| | | M252Y/S254T/T256E) | Increased | Increased half-life |
| | | | binding to FcRn | |
| hIgG1e11- | IgG1 | K322A | Reduced binding | Reduced CDC |
| Fc | | | to C1q | |
| hIgG1e12- | IgG1 | N297A + YTE | | Reduced ADCC and |
| Fc | | (N298A + | | CDC |
| | | M252Y/S254T/T256E) + | | Increased half-life |
| | | K444A | | Abolish cleveage of |
| | | | | the C-terminal lysine |
| | | | | of the antbody |
| hIgG4e1-Fc | IgG4 | S228P | — | Reduced Fab-arm |
| | | | | exchange |
| hIgG4e1-Fc | IgG4 | LALA (L234A/L235A) | Increased | Increased half-life |
| | | | binding to FcRn | |
| hIgG4e2-Fc | IgG4 | S228P + YTE (S228P + | — | Reduced Fab-arm |
| | | M252Y/S254T/T256E) | Increased | exchange |
| | | | binding to FcRn | Increased half-life |
| hIgG4e3-Fc | IgG4 | N297A + YTE | | Reduced ADCC and |
| | | (N298A + | | CDC |
| | | M252Y/S254T/T256E) + | | Increased half-life |
| | | K444A | | Abolish cleveage of |
| | | | | the C-terminal lysine |
| | | | | of the antibody |

numbering of residues in the heavy chain constant region is according to EU numbering (Edelman, G. M. et al., Proc. Natl. Acad. USA, 63, 78-85 (1969); www.imgt.org/IMGTScientificChart/Numbering/Hu_IGHGnber.html#refs)

In a particular aspect, the bifunctional molecule comprises a human IgG1 heavy chain constant domain or an IgG1 Fc domain, optionally with a substitution or a combination of substitutions selected from the group consisting of T250Q/M428L; M252Y/S254T/T256E+H433K/N434F; E233P/L234V/L235A/G236A+A327G/A330S/P331S; E333A; S239D/A330L/I332E; P257I/Q311; K326W/E333S; S239D/1332E/G236A; N297A; L234A/L235A; P329G; N297A+M252Y/S254T/T256E; K322A and K444A, preferably selected from the group consisting of N297A optionally in combination with M252Y/S254T/T256E, and L234A/L235A optionally with P329G.

The bifunctional molecule comprising a human IgG1 heavy chain constant domain or an IgG1 Fc domain with the combination of substitutions L234A/L235A/P329G greatly reduces or altogether suppresses ADCC, ADCP and/or CDC caused by said bifunctional molecule, thus reducing nonspecific cytotoxicity.

In another aspect, the bifunctional molecule comprises a human IgG4 heavy chain constant domain or a human IgG4 Fc domain, optionally with a substitution or a combination of substitutions selected from the group consisting of S228P; L234A/L235A; L234A/L235A/P329G, P329G, S228P+M252Y/S254T/T256E, K444A K444E, K444D, K444G and K444A. Even more preferably, the bifunctional molecule, preferably the binding moiety, comprises an IgG4 Fc-region with a S228P that stabilizes the IgG4.

As mentioned herein the “/” and “+” refer to mutations that are cumulative. Thus, by the mutation S228P +M252Y/S254T/T256E, it is meant the following mutations: S228P, M252Y, S254T and T256E.

The bifunctional molecule comprising a human IgG4 heavy chain constant domain or an IgG4 Fc domain with the substitution P329G reduces ADCC and/or CDC caused by said bifunctional molecule, thus reducing nonspecific cytotoxicity.

All subclass of Human IgG carries a C-terminal lysine residue of the antibody heavy chain (K444) that are susceptible to be cleaved off in circulation. This cleavage in the blood may compromise or decrease the bioactivity of the bifunctional molecule by releasing the linked immuno-stimulating cytokine to the bifunctional molecule. To circumvent this issue, K444 amino acid in the IgG domain can be substituted by an alanine to reduce proteolytic cleavage, a mutation commonly used for antibodies. Then, in one aspect, the bifunctional molecule comprises at least one further amino acid substitution consisting of K444A, K444E, K444D, K444G or K444S, preferentially K444A.

Optionally, the bifunctional molecule comprises an additional cysteine residue at the C-terminal domain of the Fc domain to create an additional disulfide bond and potentially restrict the flexibility of the bifunctional molecule.

In one aspect, the bifunctional molecule comprises one heavy chain constant domain of SEQ ID NO: 39 or 52 and/or one light chain constant domain of SEQ ID NO: 40, particularly one heavy chain constant domain or Fc domain of SEQ ID NO: 39 or 52 and one light chain constant domain of SEQ ID NO: 40, particularly such as disclosed in Table H below.

TABLE H

| | |
|---|---|
| Heavy chain | ASTKGPSVFPLAPCSRSTSESTAALGCLVKDY |
| constant domain | FPEPVTVSWNSGALTSGVHTFPAVLQSSGLYS |
| (IgG4m-S228P) | LSSVVTVPSSSLGTKTYTCNVDHKPSNTKVDK |
| SEQ ID NO: 39 | RVESKYGPPCPPCPAPEFLGGPSVFLFPPKPK |
| | DTLMISRTPEVTCVVVDVSQEDPEVQFNWYVD |
| | GVEVHNAKTKPREEQFNSTYRVVSVLTVLHQD |
| | WLNGKEYKCKVSNKGLPSSIEKTISKAKGQPR |
| | EPQVYTLPPSQEEMTKNQVSLTCLVKGFYPSD |
| | IAVEWESNGQPENNYKTTPPVLDSDGSFFLYS |
| | RLTVDKSRWQEGNVFSCSVMHEALHNHYTQKS |
| | LSLSPGK |
| Light chain | RTVAAPSVFIFPPSDEQLKSGTASVVCLLNNF |
| constant domain | YPREAKVQWKVDNALQSGNSQESVTEQDSKDS |
| (CLkappa) | TYSLSSTLTLSKADYEKHKVYACEVTHQGLSS |
| SEQ ID NO: 40 | PVTKSFNRGEC |
| Heavy chain | ASTKGPSVFPLAPSSKSTSGGTAALGCLVKDY |
| constant domain | FPEPVTVSWNSGALTSGVHTFPAVLQSSGLYS |
| (IgG1m-N298A) | LSSVVTVPSSSLGTQTYICNVNHKPSNTKVDK |
| SEQ ID NO: 52 | KVEPKSCDKTHTCPPCPAPELLGGPSVFLFPP |
| | KPKDTLMISRTPEVTCVVVDVSHEDPEVKFNW |
| | YVDGVEVHNAKTKPREEQYASTYRVVSVLTVL |
| | HQDWLNGKEYKCKVSNKALPAPIEKTISKAKG |
| | QPREPQVYTLPPSRDELTKNQVSLTCLVKGFY |
| | PSDIAVEWESNGQPENNYKTTPPVLDSDGSFF |
| | LYSKLTVDKSRWQQGNVFSCSVMHEALHNHYT |
| | QKSLSLSPGK |

Example of a heavy chain constant domain and a light chain constant domain suitable for the bifunctional molecules according to the invention.

In one particular aspect, the bifunctional molecule according to the invention comprises a heterodimer of Fc domains that comprises the “knob into holes” modifications such as described above. Preferably, such Fc domains are IgG1 or IgG4 Fc domain such as described above, even more preferably an IgG1 Fc domain comprising the mutation N297A such as disclosed above.

For instance, the first Fc chain is a “hole” or H chain and comprises the substitutions T366S/L368A/Y407V/Y349C and optionally N297A and the second Fc chain is a “knob” or K chain and comprises the substitutions T366W/S354C and optionally N297A. Preferably, the first Fc chain is a “hole” or H chain and comprises the substitutions T366S/L368A/Y407V/Y349C and N297A and the second Fc chain is a “knob” or K chain and comprises the substitutions T366W/S354C and N297A. More particularly, the second Fc chain may comprise or consists in SEQ ID NO: 75 and/or the first Fc chain may comprise or consists in SEQ ID NO: 77.

More specifically, the immuno-stimulating cytokine according to the invention is linked to the knob-chain and/or the hole chain of the heterodimeric Fc domain. Thus, the bifunctional molecule according to the invention may comprises a single immuno-stimulating cytokine either linked to the hole-chain or to the knob-chain of the Fc domain. Preferably, the bifunctional molecule according to the invention comprises a single immuno-stimulating cytokine linked to the hole-chain of the Fc domain.

In a first aspect, the bifunctional molecule comprises an immuno-stimulating cytokine linked to the C-terminal of the knob-chain of the Fc domain, such knob-chain of the Fc domain being linked to an antigen binding domain.

In a second aspect, the bifunctional molecule comprises an immuno-stimulating cytokine immuno-stimulating cytokine linked to the C-terminal of the hole-chain of the Fc domain, such hole-chain of the Fc-domain being linked to an antigen binding domain at its N-terminal end.

Optionally, the bifunctional molecule comprises a single immuno-stimulating cytokine linked to the C-terminal of the hole-chain of the Fc domain, wherein the bifunctional molecule comprises only a single antigen binding domain linked in the N-terminal end of the hole chain of the Fc domain. In such aspect, the knob chain domain is devoid of immuno-stimulating cytokine and of an antigen binding domain.

Optionally, the bifunctional molecule comprises a single immuno-stimulating cytokine linked to the C-terminal of the knob-chain of the Fc domain, wherein the bifunctional molecule comprises only a single antigen binding domain linked in the N-terminal end of the knob chain of the Fc domain. In such aspect, the hole chain domain is devoid of immuno-stimulating cytokine and of an antigen binding domain.

Accordingly, an object of the present invention relates to a polypeptide comprising from the N-terminal to the C-terminal an antigen binding domain (or at least the part therefor corresponding to the heavy chain), a Fc chain (knob or hole Fc chain), preferably the hole-chain of the Fc domain, and an immuno-stimulating cytokine. The complementary chain comprises a complementary Fc chain devoid of immuno-stimulating cytokine and of antigen binding domain, preferably the knob-chain of the Fc domain.

In a very particular aspect, the bifunctional molecule targets PD-1 and comprises:
(a) a heavy chain comprising or consisting of an amino acid sequence selected from the group consisting of SEQ ID NO: 29, 30, 31, 32, 33, 34, 35 or 36, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 29, 30, 31, 32, 33, 34, 35 or 36, respectively, and the substitutions corresponding to the hole or knob chain, preferably the hole chain, more specifically as disclosed in Table F, in particular, in SEQ ID NO: 29, 30, 31, 32, 33, 34, 35 or 36, either T363S/L365A/Y4047V/Y346C or T363W/S351C, preferably T363S/L365A/Y4047V/Y346C, and optionally N294A in any of SEQ ID NO: 29, 30, 31, 32, 33, 34, 35 or 36;
(b) a light chain comprising or consisting of an amino acid sequence of SEQ ID NO: 37 or SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37 or SEQ ID NO: 38.

In another aspect, the bifunctional molecule comprises or consists in any of the following combinations of a heavy chain (CH) and a light chain (CL):

| CH (SEQ ID NO:), optionally with one, two or | |
|---|---|
| three modification(s) selected from | |
| substitution(s), addition(s), deletion(s) at any | CL (SEQ ID NO:), optionally with one, two or |
| position but positions 7, 16, 17, 20, 33, 38, 43, | three modification(s) selected from |
| 46, 62, 63, 65, 69, 73, 76, 78, 80, 84, 85, 88, 93, | substitution(s), addition(s), deletion(s) at any |
| 95, 96, 97, 98, 100, 101, 105, 106 and 112 of | position but positions 3, 4, 7, 14, 17, 18, 28, 29, |
| SEQ ID NO: 29, 30, 31, 32, 33, 34, 35 or 36 of | 33, 34, 39, 42, 44, 50, 81, 88, 94, 97, 99 and |
| SEQ ID NO: | 105of SEQ ID NO: |
| 29 | 37 |
| 29 | 38 |
| 30 | 37 |
| 30 | 38 |
| 31 | 37 |
| 31 | 38 |
| 32 | 37 |
| 32 | 38 |
| 33 | 37 |
| 33 | 38 |
| 34 | 37 |
| 34 | 38 |
| 35 | 37 |
| 35 | 38 |
| 36 | 37 |
| 36 | 38 |

with the heavy chain comprising the substitutions corresponding to the hole or knob chain, preferably the hole chain, more specifically as disclosed in Table F, in particular, in SEQ ID NO: 29, 30, 31, 32, 33, 34, 35 or 36, in particular either T366S/L368A/Y407V/Y349C or T366W/S354C, preferably T366S/L368A/Y407V/Y349C, and optionally N297A in any of SEQ ID NO: 29, 30, 31, 32, 33, 34, 35 or 36, the positions of the substitutions being defined according to EU numbering.

In a very particular aspect, the bifunctional molecule targets PD-1 and comprises a light chain comprising or consisting of SEQ ID NO: 37 or 38.

Optionally, the heavy chain may comprise a peptide signal, for example such as described under SEQ ID NO: 49. Preferably, such peptide signal is comprised at the N-terminal of the heavy chain.

Optionally, the light chain may comprise a peptide signal, for example such as described under SEQ ID NO: 50. Preferably, such peptide signal is comprised at the N-terminal of the light chain.

Accordingly, the bifunctional molecule may comprise one heavy chain comprising any of the SEQ ID NOs: 29, 30, 31, 32, 33, 34, 35 and 36, the Fc chain being optionally modified to promote a heterodimerization of the Fc chains for forming a heterodimeric Fc domain. More specifically, the heavy chain comprises the substitutions corresponding to the hole or knob chain, preferably the hole chain, more specifically as disclosed in Table F, particularly either T366S/L368A/Y407V/Y349C or T366W/S354C, preferably T366S/L368A/Y407V/Y349C, and optionally N297A in any of SEQ ID NO: 29, 30, 31, 32, 33, 34, 35 or 36, the positions of the substitutions being defined according to EU numbering. The heavy chain is linked, optionally via a linker, at its C terminal end to the immuno-stimulating cytokine.

In a very particular aspect, the bifunctional molecule comprises a light chain comprising or consisting of SEQ ID NO: 38 and one heavy chain comprising SEQ ID NO: 35, the Fc chain being optionally modified to promote a heterodimerization of the Fc chains for forming a heterodimeric Fc domain. In one aspect, the heavy chain is linked, optionally via a linker, at its C terminal end to the immuno-stimulating cytokine. In an alternative aspect, the light chain is linked, optionally via a linker, at its C terminal end to the immuno-stimulating cytokine.

In a very particular aspect, the bifunctional molecule may comprise a first monomer of SEQ ID NO: 75 and a second monomer comprising a Fc chain SEQ ID NO: 77, to which is linked at the N-terminal end, optionally by a linker, to an antigen binding domain (for instance of SEQ ID NO: 79). More preferably, the bifunctional molecule may comprise a first monomer of SEQ ID NO: 75 and a second monomer comprising a Fc chain SEQ ID NO: 77, to which is linked at the N-terminal end, optionally by a linker, to an antigen binding domain (for instance of SEQ ID NO: 79), and at the C-terminal end, optionally by a linker, to any immuno-stimulating cytokine as disclosed herein.

Optionally, the immuno-stimulating cytokine can be selected from the group consisting of IL-2 (SEQ ID NO: 87), IL-15 (SEQ ID NO: 88), IL-21 (SEQ ID NO: 89) or a variant thereof having at least 80, 85, 90, 91, 92, 93, 94, 95, 96, 97, 98 or 99% of identity therewith or having 1 to 10 modifications selected from the group consisting of addition, deletion, substitution and combinations thereof with respect to the wildtype protein. Optionally, the immuno-stimulating cytokine can be IL-7 (SEQ ID NO: 1).

Optionally, when the immuno-stimulating cytokine is IL-2, the bifunctional molecule may comprise a first monomer of SEQ ID NO: 75, a second monomer of SEQ ID NO: 84, and a third monomer of SEQ ID NO: 37, 38 or 80, preferably SEQ ID NO: 38 or 80.

Optionally, when the immuno-stimulating cytokine is IL-2, the bifunctional molecule may comprise a first monomer of SEQ ID NO: 75, a second monomer of SEQ ID NO: 90, and a third monomer of SEQ ID NO: 37, 38 or 80, preferably SEQ ID NO: 38 or 80, linked at its terminal end, optionally by a linker, to IL-2 of SEQ ID NO: 87 or a variant thereof.

Optionally, when the immuno-stimulating cytokine is IL-7, the bifunctional molecule may comprise a first monomer of SEQ ID NO: 75, a second monomer of SEQ ID NO: 93, and a third monomer of SEQ ID NO: 37, 38 or 80, preferably SEQ ID NO: 38 or 80.

Optionally, when the immuno-stimulating cytokine is IL-7, the bifunctional molecule may comprise a first monomer of SEQ ID NO: 75, a second monomer of SEQ ID NO: 90, and a third monomer of SEQ ID NO: 37, 38 or 80, preferably SEQ ID NO: 38 or 80, linked at its terminal end, optionally by a linker, to IL-7 of SEQ ID NO: 1.

Optionally, when the immuno-stimulating cytokine is IL-15, the bifunctional molecule may comprise a first monomer of SEQ ID NO: 75, a second monomer of SEQ ID NO: 85, and a third monomer of SEQ ID NO: 37, 38 or 80, preferably SEQ ID NO: 38 or 80.

Optionally, when the immuno-stimulating cytokine is IL-15, the bifunctional molecule may comprise a first monomer of SEQ ID NO: 75, a second monomer of SEQ ID NO: 90, and a third monomer of SEQ ID NO: 37, 38 or 80, preferably SEQ ID NO: 38 or 80, linked at its terminal end, optionally by a linker, to IL-15 of SEQ ID NO: 88 or a variant thereof.

Optionally, when the immuno-stimulating cytokine is IL-21, the bifunctional molecule may comprise a first monomer of SEQ ID NO: 75, a second monomer of SEQ ID NO: 86, and a third monomer of SEQ ID NO: 37, 38 or 80, preferably SEQ ID NO: 38 or 80.

Optionally, when the immuno-stimulating cytokine is IL-21, the bifunctional molecule may comprise a first monomer of SEQ ID NO: 75, a second monomer of SEQ ID NO: 90, and a third monomer of SEQ ID NO: 37, 38 or 80, preferably SEQ ID NO: 38 or 80, linked at its terminal end, optionally by a linker, to IL-21 of SEQ

ID NO: 89 or a variant thereof.

In another very particular aspect, the bifunctional molecule may comprise a first monomer of SEQ ID NO: 77 and a second monomer comprising a Fc chain SEQ ID NO: 75, to which is linked at the N-terminal end, optionally by a linker, to an antigen binding domain (for instance of SEQ ID NO: 79), and at the C-terminal end, optionally by a linker, to any immuno-stimulating cytokine as disclosed herein.

In another very particular aspect, the bifunctional molecule may comprise a first monomer of SEQ ID NO: 77, a second monomer comprising a Fc chain SEQ ID NO: 75, to which is linked at the N-terminal end, optionally by a linker, to an antigen binding domain (for instance of SEQ ID NO: 79), and a third monomer of SEQ ID NO: 37, 38 or 80, preferably SEQ ID NO: 38 or 80, linked at its terminal end, optionally by a linker, to any immuno-stimulating cytokine as disclosed herein.

Optionally, the immuno-stimulating cytokine can be selected from the group consisting of IL-2 (SEQ ID NO: 87), IL-15 (SEQ ID NO: 88), and IL-21 (SEQ ID NO: 89), or a variant thereof having at least 80, 85, 90, 91, 92, 93, 94, 95, 96, 97, 98 or 99% of identity therewith or having 1 to 10 modifications selected from the group consisting of addition, deletion, substitution and combinations thereof with respect to the wildtype protein. Optionally, the immuno-stimulating cytokine can be IL-7 (SEQ ID NO: 1).

### Preparation of Bifunctional Molecule—Nucleic Acid Molecules Encoding the Bifunctional Molecules of the Present Invention, Recombinant Expression Vectors and Host Cells Comprising Such

To produce a bifunctional molecule according to the invention, in particular by mammalian cells, nucleic acid sequences or group of nucleic acid sequences coding for the bifunctional molecule are subcloned into one or more expression vectors. Such vectors are generally used to transfect mammalian cells. General techniques for producing molecules comprising antibody sequences are described in Coligan et al. (eds.), Current protocols in immunology, at pp. 10.19.1-10.19.11 (Wiley Interscience 1992), the contents of which are hereby incorporated by reference and in “Antibody engineering: a practical guide” from W. H. Freeman and Company (1992), in which commentary relevant to production of molecules is dispersed throughout the respective texts.

Generally, such method comprises the following steps of:
(1) transfecting or transforming appropriate host cells with the polynucleotide(s) encoding the recombinant bifunctional molecule of the invention or the vector containing the polynucleotide(s);
(2) culturing the host cells in an appropriate medium; and
(3) optionally isolating or purifying the bifunctional molecule from the medium or host cells.

The invention further relates to a nucleic acid encoding a bifunctional molecule as disclosed above, a vector, preferably an expression vector, comprising the nucleic acid of the invention, a genetically engineered host cell transformed with the vector of the invention or directly with the sequence encoding the recombinant bifunctional molecule, and a method for producing the bifunctional molecule of the invention by recombinant techniques.

The nucleic acid, the vector and the host cells are more particularly described hereafter.

### Nucleic Acid Sequence

The invention also relates to a nucleic acid molecule encoding the bifunctional molecule as defined above or to a group of nucleic acid molecules encoding the bifunctional molecule as defined above. Nucleic acid encoding the bifunctional molecule disclosed herein can be amplified by any techniques known in the art, such as PCR. Such nucleic acid may be readily isolated and sequenced using conventional procedures. Particularly, the nucleic acid molecules encoding the bifunctional molecule as defined herein comprises:
a first nucleic acid molecule encoding the first monomer comprising an antigen-binding domain covalently linked to a first Fc chain optionally via a peptide linker, said first Fc chain being covalently linked to the immuno-stimulating cytokine, optionally via a peptide linker, and
a second nucleic acid molecule encoding the second monomer comprising a complementary second Fc chain,
optionally a third nucleic acid molecule encoding the light chain of the antigen-binding domain.

In an alternative aspect, the nucleic acid molecules encoding the bifunctional molecule as defined herein comprises:
a first nucleic acid molecule encoding the first monomer comprising an antigen-binding domain covalently linked to a first Fc chain optionally via a peptide linker,
a second nucleic acid molecule encoding the second monomer comprising a complementary second Fc chain, and
a third nucleic acid molecule encoding the light chain of the antigen-binding domain, said light chain being covalently linked to the immuno-stimulating cytokine, optionally via a peptide linker.

In one embodiment, the nucleic acid molecule is an isolated, particularly non-natural, nucleic acid molecule.

### Vectors

In another aspect, the invention relates to a vector comprising the nucleic acid molecule or the group of nucleic acid molecules as defined above.

As used herein, a “vector” is a nucleic acid molecule used as a vehicle to transfer genetic material into a cell. The term “vector” encompasses plasmids, viruses, cosmids and artificial chromosomes. In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The nucleic acid molecule encoding the bifunctional molecule can be cloned into a vector by those skilled in the art, and then transformed into host cells. These methods include in vitro recombinant DNA techniques, DNA synthesis techniques, in vivo recombinant techniques, etc. The methods known to the artisans in the art can be used to construct an expression vector containing the nucleic acid sequence of described herein and appropriate regulatory components for the bifunctional molecule transcription/translation.

Accordingly, the present invention also provides a recombinant vector, which comprises a nucleic acid molecule encoding the bifunctional molecule according to the present invention. In one preferred aspect, the expression vector further comprises a promoter and a nucleic acid sequence encoding a secretion signal peptide, and optionally at least one drug-resistance gene for screening. The expression vector may further comprise a ribosome -binding site for initiating the translation, transcription terminator and the like.

Suitable expression vectors typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence.

An expression vector can be introduced into host cells using a variety of techniques including calcium phosphate transfection, liposome-mediated transfection, electroporation, and the like. Preferably, transfected cells are selected and propagated wherein the expression vector is stably integrated in the host cell genome to produce stable transformants.

### Host Cells

In another aspect, the invention relates to a host cell comprising a vector or a nucleic acid molecule or group of nucleic acid molecules as defined above, for example for bifunctional molecule production purposes.

As used herein, the term “host cell” is intended to include any individual cell or cell culture that can be or has been recipient of vectors, exogenous nucleic acid molecules, and polynucleotides encoding the bifunctional molecule according to the present invention. The term “host cell” is also intended to include progeny or potential progeny of a single cell. Suitable host cells include prokaryotic or eukaryotic cells, and also include but are not limited to bacteria, yeast cells, fungi cells, plant cells, and animal cells such as insect cells and mammalian cells, e.g., murine, rat, rabbit, macaque or human.

Suitable hosts cells are especially eukaryotic hosts cells which provide suitable post-translational modifications such as glycosylation. Preferably, such suitable eukaryotic host cell may be fungi such as Pichia pastoris, Saccharomyces cerevisiae, Schizosaccharomyces pombe; insect cell such as Mythimna separate; plant cell such as tobacco, and mammalian cells such as BHK cells, 293 cells, CHO cells, NSO cells and COS cells.

Preferably, the host cell of the present invention is selected from the group consisting of CHO cell, COS cell, NSO cell, and HEK cell.

Then host cells stably or transiently express the bifunctional molecule according to the present invention. Such expression methods are known by the man skilled in the art.

A method of production of the bifunctional molecule is also provided herein. The method comprises culturing a host cell comprising a nucleic acid encoding the bifunctional molecule as provided above, under conditions suitable for its expression, and optionally recovering the bifunctional molecule from the host cell (or host cell culture medium). Particularly, for recombinant production of a bifunctional molecule, nucleic acid encoding a bifunctional molecule, e.g., as described above, is isolated and inserted into one or more vectors for further cloning and/or expression in a host cell. The bifunctional molecules are then isolated and/or purified by any methods known in the art. These methods include, but are not limited to, conventional renaturation treatment, treatment by protein precipitant (such as salt precipitation), centrifugation, cell lysis by osmosis, sonication, supercentrifugation, molecular sieve chromatography or gel chromatography, adsorption chromatography, ion exchange chromatography, HPLC, any other liquid chromatography, and the combination thereof. As described, for example, by Coligan, bifunctional molecule isolation techniques may particularly include affinity chromatography with Protein-A Sepharose, size-exclusion chromatography and ion exchange chromatography. Protein A preferably is used to isolate the bifunctional molecules of the invention.

### Pharmaceutical Composition and Method of Administration Thereof

The present invention also relates to a pharmaceutical composition comprising a bifunctional molecule described herein, the nucleic acid molecule, the group of nucleic acid molecules, the vector and/or the host cells as described hereabove, preferably as the active ingredient or compound. The formulations can be sterilized and, if desired, mixed with auxiliary agents such as pharmaceutically acceptable carriers, excipients, salts, anti-oxidant and/or stabilizers which do not deleteriously interact with the bifunctional molecule of the invention, nucleic acid, vector and/or host cell of the invention and does not impart any undesired toxicological effects. Optionally, the pharmaceutical composition may further comprise an additional therapeutic agent.

Particularly, the pharmaceutical composition according to the invention can be formulated for any conventional route of administration including a topical, enteral, oral, parenteral, intranasal, intravenous, intramuscular, subcutaneous or intraocular administration and the like. To facilitate administration, the bifunctional molecule as described herein can be made into a pharmaceutical composition for in vivo administration. The means of making such a composition have been described in the art (see, for instance, Remington: The Science and Practice of Pharmacy, Lippincott Williams & Wilkins, 21st edition (2005).

The pharmaceutical composition may be prepared by mixing a bifunctional molecule having the desired degree of purity with optional pharmaceutically acceptable carriers, excipients, anti-oxidant, and/or stabilizers in the form of lyophilized formulations or aqueous solutions. Such suitable carriers, excipients, anti-oxidant, and/or stabilizers are well known in the art and have been for example described in Remington's Pharmaceutical Sciences 16th edition, Osol, A. Ed. (1980).

To facilitate delivery, any of the bifunctional molecule or its encoding nucleic acids can be conjugated with a chaperon agent. The chaperon agent can be a naturally occurring substance, such as a protein (e.g., human serum albumin, low-density lipoprotein, or globulin), carbohydrate (e.g., a dextran, pullulan, chitin, chitosan, inulin, cyclodextrin or hyaluronic acid), or lipid. It can also be a recombinant or synthetic molecule, such as a synthetic polymer, e.g., a synthetic polypeptide.

Pharmaceutical compositions according to the invention may be formulated to release the active ingredients (e.g. the bifunctional molecule of the invention) substantially immediately upon administration or at any predetermined time or time period after administration. The pharmaceutical composition in some aspects can employ time-released, delayed release, and sustained release delivery systems such that the delivery of the composition occurs prior to, and with sufficient time to cause, sensitization of the site to be treated. Means known in the art can be used to prevent or minimize release and absorption of the composition until it reaches the target tissue or organ, or to ensure timed-release of the composition. Such systems can avoid repeated administrations of the composition, thereby increasing convenience to the subject and the physician.

It will be understood by one skilled in the art that the formulations of the invention may be isotonic with human blood that is the formulations of the invention have essentially the same osmotic pressure as human blood. Such isotonic formulations generally have an osmotic pressure from about 250 mOSm to about 350 mOSm. Isotonicity can be measured by, for example, a vapor pressure or ice-freezing type osmometer.

Pharmaceutical composition typically must be sterile and stable under the conditions of manufacture and storage. Prevention of presence of microorganisms may be ensured both by sterilization procedures (for example by microfiltration), and/or by the inclusion of various antibacterial and antifungal agents

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect.

### Subject, Regimen and Administration

The present invention relates to a bifunctional molecule as disclosed herein, a nucleic acid or a vector encoding such, a host cell or a pharmaceutical composition for use as a medicament or for use in the treatment of a disease or for administration in a subject or for use as a medicament. It also relates to a method for treating a disease or a disorder in a subject comprising administering a therapeutically effective amount of a pharmaceutical composition or a bifunctional molecule to a subject.

The subject to treat may be a human, particularly a human at the prenatal stage, a new-born, a child, an infant, an adolescent or an adult, in particular an adult of at least 30 years old, 40 years old, preferably an adult of at least 50 years old, still more preferably an adult of at least 60 years old, even more preferably an adult of at least 70 years old.

In a particular aspect, the subject can be immunosuppressed or immunocompromised.

Conventional methods, known to those of ordinary skill in the art of medicine, can be used to administer the bifunctional molecule or the pharmaceutical composition disclosed herein to a subject, depending upon the type of diseases to be treated or the site of the disease e.g., administered orally, parenterally, enterally, by inhalation spray, topically, rectally, nasally, buccally, vaginally or via an implanted reservoir. Preferably, the bifunctional molecule or the pharmaceutical composition is administered via subcutaneous, intra-cutaneous, intravenous, intramuscular, intra-articular, intra-arterial, intra-synovial, intra-tumoral, intra-sternal, intra-thecal, intra-lesion, and intracranial injection or infusion techniques.

The form of the pharmaceutical compositions, the route of administration and the dose of administration of the pharmaceutical composition or the bifunctional molecule according to the invention can be adjusted by the man skilled in the art according to the type and severity of the infection, and to the patient, in particular its age, weight, size, sex, and/or general physical condition. The compositions of the present invention may be administered in a number of ways depending upon whether local or systemic treatment is desired.

### Use in the Treatment of a Disease

The bifunctional molecules, nucleic acids, vectors, host cells, compositions and methods of the present invention have numerous in vitro and in vivo utilities and applications. Particularly, any of bifunctional molecules, nucleic acid molecules, group of nucleic acid molecules, vectors, host cells or pharmaceutical composition provided herein may be used in therapeutic methods and/or for therapeutic purposes.

The present invention also relates to a bifunctional molecule, a nucleic acid or a vector encoding such, or a pharmaceutical composition comprising such for use in the treatment of a disorder and/or disease in a subject and/or for use as a medicament or vaccine. It also relates to the use of a bifunctional molecule as described herein; a nucleic acid or a vector encoding such, or a pharmaceutical composition comprising such for treating a disease and/or disorder in a subject. Finally, it relates to a method for treating a disease or a disorder in a subject comprising administering a therapeutically effective amount of a pharmaceutical composition or a bifunctional molecule to the subject, or a nucleic acid or a vector encoding such.

In one aspect, the invention relates to a method of treatment of a disease and/or disorder selected from the group consisting of a cancer, an infectious disease and a chronic viral infection in a subject in need thereof comprising administering to said subject an effective amount of a bifunctional molecule or pharmaceutical composition as defined above. Examples of such diseases are more particularly described hereafter.

In one aspect, the treatment method comprises: (a) identifying a patient in need of treatment; and (b) administering to the patient a therapeutically effective amount of a bifunctional molecule, nucleic acid, vector or pharmaceutical composition as described herein.

A subject in need of a treatment may be a human having, at risk for, or suspected of having a disease. Such a patient can be identified by routine medical examination.

In another aspect, the bifunctional molecules disclosed herein can be administered to a subject, e.g., in vivo, to enhance immunity, preferably in order to treat a disorder and/or disease. Accordingly, in one aspect, the invention provides a method of modifying an immune response in a subject comprising administering to the subject a bifunctional molecule, nucleic acid, vector or pharmaceutical composition of the invention such that the immune response in the subject is modified. Preferably, the immune response is enhanced, increased, stimulated or up-regulated. The bifunctional molecule or pharmaceutical composition can be used to enhance immune responses such as T cell activation in a subject in need of a treatment. In a particular embodiment, the bifunctional molecule or pharmaceutical composition can be used to reduce T cells exhaustion or to reactivate exhausted T cells.

The invention particularly provides a method of enhancing an immune response in a subject, comprising administering to the subject a therapeutic effective amount of any of the bifunctional molecule, nucleic acid, vector or pharmaceutical composition comprising such described herein, such that an immune response in the subject is enhanced. In a particular embodiment, the bifunctional molecule or pharmaceutical composition can be used to reduce T cells exhaustion or to reactivate exhausted T cells.

Bifunctional molecules including IL-7 according to the invention target CD127+ immune cells, particularly CD127+ T cells. Such cells may be found in the following areas of particular interest: resident lymphoid cells in the lymph nodes (mainly within paracortex, with occasional cells in follicles), in tonsil (inter-follicular areas), spleen (mainly within the Peri-Arteriolar Lymphoid Sheaths (PALS) of the white pulp and some scattered cells in the red pulp), thymus (primarily in medulla; also in cortex), bone marrow (scattered distribution), in the GALT (Gut Associated-Lymphoid-Tissue, primarily in inter-follicular areas and lamina propria) throughout the digestive tract (stomach, duodenum, jejunum, ileum, cecum colon, rectum), in the MALT (Mucosa-Associated-Lymphoid-Tissue) of the gall bladder. Therefore, the bifunctional molecules of the invention are of particular interest for treating diseases located or involving these areas, in particular cancers.

Such a bifunctional molecule and a pharmaceutical composition comprising it can be for use in a patient, in particular a patient having a cancer, for increasing Tumor Infiltrating lymphocytes (TILs), protecting T lymphocytes from apoptosis, inducing/improving T memory response, abrogation of T-reg suppression and/or suppressive activity of Treg, restoring proliferation and/or maintaining fully exhausted T cells, especially exhausted Tumor Infiltrating lymphocytes.

Such a bifunctional molecule and a pharmaceutical composition comprising it can be used for the manufacture of a medicine for increasing Tumor Infiltrating lymphocytes (TILs), protecting T lymphocytes from apoptosis, inducing/improving T memory response, abrogation of T-reg suppression and/or suppressive activity of Treg, restoring proliferation and/or maintaining fully exhausted T cells, especially exhausted Tumor Infiltrating lymphocytes, in a patient, in particular a patient having a cancer.

The present invention also relates to a method for increasing Tumor Infiltrating lymphocytes (TILs), protecting T lymphocytes from cell death, inducing/improving T memory response, abrogation of T-reg suppression and/or suppressive activity of Treg, restoring proliferation and/or maintaining fully exhausted T cells, especially exhausted Tumor Infiltrating lymphocytes, in a patient, in particular a patient having a cancer, comprising administering to said patient a therapeutically effective amount of a bifunctional molecule including an IL-7, and an antigen binding domain that binds and antagonizes PD-1, in particular any of such a particular molecule disclosed herein.

### Cancer

In another aspect, the invention provides the use of a bifunctional molecule or pharmaceutical composition as disclosed herein in the manufacture of a medicament for treating a cancer, for instance for inhibiting growth of tumor cells in a subject.

The term “cancer” as used herein is defined as disease characterized by the rapid and uncontrolled growth of aberrant cells. Cancer cells can spread locally or through the bloodstream and lymphatic system to other parts of the body.

Accordingly, in one aspect, the invention provides a method of treating a cancer, for instance for inhibiting growth of tumor cells, in a subject, comprising administering to the subject a therapeutically effective amount of bifunctional molecule or pharmaceutical composition according to the invention. Particularly, the present invention relates to the treatment of a subject using a bifunctional molecule such that growth of cancerous cells is inhibited.

In an aspect of the disclosure, the cancer to be treated is associated with exhausted T cells.

Any suitable cancer may be treated with the provided herein can be hematopoietic cancer or solid cancer. Such cancers include carcinoma, cervical cancer, colorectal cancer, esophageal cancer, gastric cancer, gastrointestinal cancer, head and neck cancer, kidney cancer, liver cancer, lung cancer, lymphoma, glioma, mesothelioma, melanoma, stomach cancer, urethral cancer environmentally induced cancers and any combinations of said cancers. Additionally, the invention includes refractory or recurrent malignancies. Preferably, the cancer to be treated or prevented is selected from the group consisting of metastatic or not metastatic, Melanoma , malignant mesothelioma, Non-Small Cell Lung Cancer, Renal Cell Carcinoma, Hodgkin's Lymphoma, Head and Neck Cancer, Urothelial Carcinoma, Colorectal Cancer, Hepatocellular Carcinoma, Small Cell Lung Cancer Metastatic Merkel Cell Carcinoma, Gastric or Gastroesophageal cancers and Cervical Cancer.

In a particular aspect, the cancer is a hematologic malignancy or a solid tumor. Such a cancer can be selected from the group consisting of hematolymphoid neoplasms, angioimmunoblastic T cell lymphoma, myelodysplasic syndrome, acute myeloid leukemia.

In a particular aspect, the cancer is a cancer induced by virus or associated with immunodeficiency. Such a cancer can be selected from the group consisting of Kaposi sarcoma (e.g., associated with Kaposi sarcoma herpes virus); cervical, anal, penile and vulvar squamous cell cancer and oropharyndeal cancers (e.g., associated with human papilloma virus); B cell non-Hodgkin lymphomas (NHL) including diffuse large B-cell lymphoma, Burkitt lymphoma, plasmablastic lymphoma, primary central nervous system lymphoma, HHV-8 primary effusion lymphoma, classic Hodgkin lymphoma, and lymphoproliferative disorders (e.g., associated with Epstein-Barr virus (EBV) and/or Kaposi sarcoma herpes virus); hepatocellular carcinoma (e.g., associated with hepatitis B and/or C viruses); Merkel cell carcinoma (e.g., associated with Merkel cell polyoma virus (MPV)); and cancer associated with human immunodeficiency virus infection (HIV) infection.

Preferred cancers for treatment include cancers typically responsive to immunotherapy. Alternatively, preferred cancers for treatment are cancers non-responsive to immunotherapy.

### Infectious Disease

The bifunctional molecule, nucleic acid, group of nucleic acid, vector, host cells or pharmaceutical compositions of the invention can be used to treat patients that have been exposed to particular toxins or pathogens. Accordingly, an aspect of the invention provides a method of treating an infectious disease in a subject comprising administering to the subject a bifunctional molecule according to the present invention, or a pharmaceutical composition comprising such, preferably such that the subject is treated for the infectious disease.

Any suitable infection may be treated with a bifunctional molecule, nucleic acid, group of nucleic acid, vector, host cells or pharmaceutical composition as provided herein.

Some examples of pathogenic viruses causing infections treatable by methods of the invention include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Some examples of pathogenic bacteria causing infections treatable by methods of the invention include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lymes disease bacteria.

Some examples of pathogenic fungi causing infections treatable by methods of the invention include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Some examples of pathogenic parasites causing infections treatable by methods of the invention include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

### Combined Therapy

The bifunctional molecule according to the invention can be combined with some other potential strategies for overcoming immune evasion mechanisms with agents in clinical development or already on the market (see table 1 from Antonia et al. Immuno-oncology combinations: a review of clinical experience and future prospects. Clin. Cancer Res. Off. J. Am. Assoc. Cancer Res. 20, 6258-6268, 2014). Such combination with the bifunctional molecule according to the invention may be useful notably for:
1—Reversing the inhibition of adaptive immunity (blocking T-cell checkpoint pathways);
2—Switching on adaptive immunity (promoting T-cell costimulatory receptor signaling using agonist molecules, in particular antibodies),
3—Improving the function of innate immune cells;
4—Activating the immune system (potentiating immune-cell effector function), for example through vaccine-based strategies.

Accordingly, also provided herein are combined therapies with any of the bifunctional molecule or pharmaceutical composition comprising such, as described herein and a suitable second agent, for the treatment of a disease or disorder. In an aspect, the bifunctional molecule and the second agent can be present in a unique pharmaceutical composition as described above. Alternatively, the terms “combination therapy” or “combined therapy”, as used herein, embrace administration of these two agents (e.g., a bifunctional molecule as described herein and an additional or second suitable therapeutic agent) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route. The agents can be administered by the same route or by different routes. For example, a first agent (e.g., a bifunctional molecule) can be administered orally, and an additional therapeutic agent (e.g., an anti-cancer agent, an anti-infection agent; or an immune modulator) can be administered intravenously. Alternatively, an agent of the combination selected may be administered by intravenous injection while the other agents of the combination may be administered orally.

In an aspect, the additional therapeutic agent can be selected in the non-exhaustive list comprising alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

For instance, the additional therapeutic agent can be selected in the group consisting of chemotherapy, radiotherapy, targeted therapy, antiangiogenic agents, hypomethylating agents, cancer vaccines, epitopes or neoepitopes from tumor antigens, myeloid checkpoints inhibitors, other immunotherapies, and HDAC inhibitors.

In an embodiment, the invention relates to a combined therapy as defined above, wherein the second therapeutic agent is particularly selected from the group consisting of therapeutic vaccines, immune checkpoint blockers or activators, in particular of adaptive immune cells (T and B lymphocytes) and antibody-drug conjugates. Preferably, suitable agents for co-use with any of the anti-hPD-1 antibodies or fragment thereof or with the pharmaceutical composition according to the invention include an antibody binding to a co-stimulatory receptor (e.g., OX40, CD40, ICOS, CD27, HVEM or GITR), an agent that induces immunogenic cell death (e.g., a chemotherapeutic agent, a radio-therapeutic agent, an anti-angiogenic agent, or an agent for targeted therapies), an agent that inhibits a checkpoint molecule (e.g., CTLA4, LAG3, TIM3, B7H3, B7H4, BTLA, or TIGIT), a cancer vaccine, an agent that modifies an immunosuppressive enzyme (e.g., IDO1 or iNOS), an agent that targets Treg cells, an agent for adoptive cell therapy, or an agent that modulates myeloid cells.

In an embodiment, the invention relates to a combined therapy as defined above, wherein the second therapeutic agent is an immune checkpoint blocker or activator of adaptive immune cells (T and B lymphocytes) selected from the group consisting of anti-CTLA4, anti-CD2, anti-CD28, anti-CD40, anti-HVEM, anti-BTLA, anti-CD160, anti-TIGIT, anti-TIM-1/3, anti-LAG-3, anti-2B4, and anti-OX40, anti-CD40 agonist, CD40-L, TLR agonists, anti-ICOS, ICOS-L and B-cell receptor agonists.

The present invention also relates to a method for treating a disease in a subject comprising administering to said subject a therapeutically effective amount of the bifunctional molecule or the pharmaceutical composition described herein and a therapeutically effective amount of an additional or second therapeutic agent.

Specific examples of additional or second therapeutic agents are provided in WO 2018/053106, pages 36-43.

In a preferred embodiment, the second therapeutic agent is selected from the group consisting of chemotherapeutic agents, radiotherapy agents, immunotherapeutic agents, cell therapy agents (such as CAR-T cells), antibiotics and probiotics.

Combination therapy could also rely on the combination of the administration of bifunctional molecule with surgery.

### EXAMPLES

### Example 1: Anti PD-1 IL-7 Molecules with One IL-7 W142H Cytokine and One Anti PD-1 Arm Demonstrated a High Efficacy to Promote cis Activity into PD-1+ IL-7R+ Cells

The inventors designed and compared the biological activity of multiple structures of bifunctional molecules comprising one or two anti PD-1 binding domains and one or two IL7 W142H mutants as described in FIG. 1. W142H substitution corresponds to a substitution of the amino acid at position 142 in the sequence as set forth in SEQ ID No: 1.

Construction 1 comprises two anti PD-1 antigen binding domains and two IL-7 W142H variants (construction 1 is also called anti PD-1*2 IL-7 W142H*2). This molecule is also called BICKI-IL-7 W142H. In the examples, a control molecule called BICKI-IL-7 WT corresponds to construction 1 but with wild type IL-7.

Construction 2 comprises two anti PD-1 antigen binding domains and a single IL-7 W142H variant (construction 2 is also called anti PD-1*2 IL-7 W142H*1).

Construction 3 comprises a single anti PD-1 antigen binding domain and a single IL-7 W142H variant (construction 3 is also called anti PD-1*1 IL-7 W142H*1). A control construction called anti-PD-1*1 is similar than construction 3 but devoid of IL-7 variant.

Construction 4 comprises a single anti PD-1 antigen binding domain and two IL-7 W142H variants (construction 4 is also called anti PD-1*1 IL- W142H*2).

Constructions 2, 3 and 4 were engineered with an IgG1 N298A isotype and amino acid sequences were mutated in the Fc portion in order to create a knob on the CH2 and CH3 of the Heavy chains A and a hole on the CH2 and CH3 of the Heavy chains B.

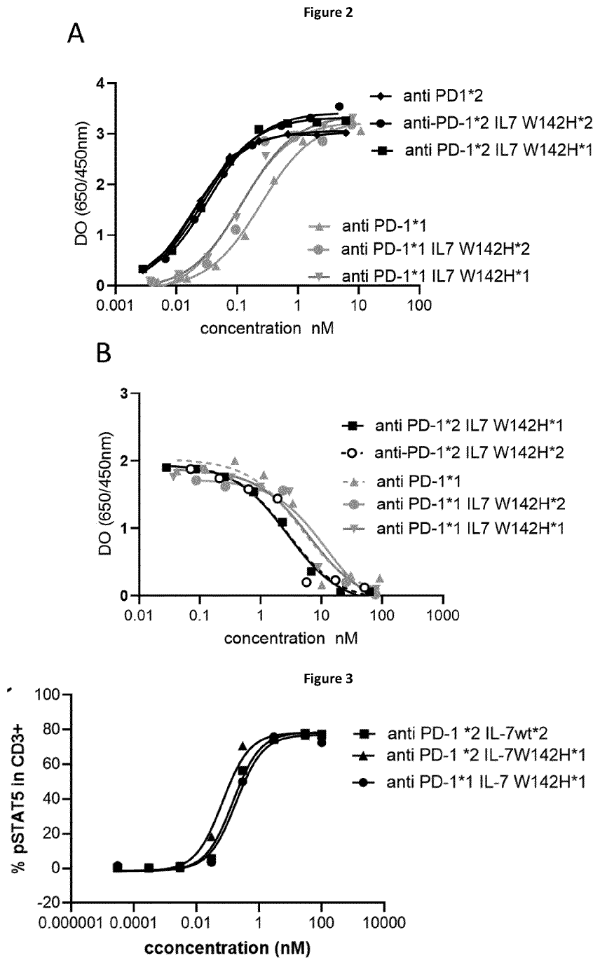

All anti PD-1 IL7 constructions possess a high affinity to PD-1 receptor as demonstrated by ELISA assay (FIG. 2A and Table 1). Anti PD-1 IL-7 molecules having 2 anti PD-1 arms (anti-PD-1*2) have the same binding efficacy (equal EC50) compared to anti PD-1*2 without IL-7. Similarly, anti PD-1 IL-7 molecules having 1 anti PD-1 arm (Anti PD-1*1 IL7 W142H*1 and anti PD-1*1 IL7 W142H*2) demonstrated the same binding efficacy compared to the anti PD-1*1 without IL-7, with an EC50 equal to 0.086 and 0.111 nM for anti PD-1 IL7 versus 0.238 nM for the anti PD-1. These data show that fusion of IL-7 does not interfere with the PD-1 binding regardless of the construction tested.

TABLE 1
ED50 determination from FIG. 2A refers to the concentration
required to reach 50% of the PD1 binding signal as measured
by ELISA for each anti PD-1 IL-7 molecule.

| Samples | EC50 (nM) |
|---|---|
| anti PD-1*2 | 0.021 |
| anti-PD-1*2 IL7 W142H*1 | 0.026 |
| anti-PD-1*2 IL7 W142H*2 | 0.034 |
| anti-PD-1*1 | 0.238 |
| anti-PD-1*1 IL7 W142H*1 | 0.111 |
| anti-PD-1*1 IL7 W142H*2 | 0.086 |

Moreover, PD-L1/PD-1 antagonist bioassay (FIG. 2B) demonstrates that anti PD-1 IL7 molecules having 1 or 2 anti PD-1 arms display high efficiency to block the binding of PD-L1 to the PD-1 receptor. Although one arm of anti PD-1 was removed from the constructions 3 and 4, all the anti PD-1*1 IL7 construction demonstrates high antagonist properties. Only a 2.5-fold decreased activity compared to anti PD-1*2 IL7 constructions was calculated with EC50 (Table 2) for the constructions 3 and 4.

TABLE 2
ED50 determination from FIG. 2B refers to the concentration
required to reach 50% of the PD1/PDL1 antagonist activity
as measured by ELISA for each anti PD-1 IL-7 molecule.

| Samples | EC50 (nM) |
|---|---|
| anti PD-1*2 IL7 W142H*1 | 2.168 |
| anti PD-1*2 IL7 W142H*2 | 2.792 |
| anti PD-1*1 | 5.014 |
| anti PD-1*1 IL7 W142H *1 | 5.839 |
| anti PD-1*1 IL7 W142H *2 | 7.235 |

The inventors next assessed the affinity of the different constructions to CD127 receptor using Biacore assay and ELISA assay. Since one IL-7 molecule was removed from construction 2 and 3, a lower binding capacity to CD127 receptor and a lower pSTAT5 activation was expected for these molecules in comparison to the IL-7 heterodimeric constructions. However, the inventors observed that the anti PD-1*2 IL-7 W142H*1 molecule has similar affinity to CD127 receptor compared to the anti PD-1*2 IL-7 W142H*2 (BICKI-IL-7 W142H) and as expected a lower affinity compared to the anti PD-1 IL7 bifunctional molecules comprising IL-7 wild type form (Table 3). Surprisingly, the anti PD-1*2 IL7 W142H *1 and the anti PD-1*1 IL7 W142H *1 molecules demonstrated a high pSTAT5 activity similar to the PD-1 IL7 bifunctional molecules comprising IL-7 wild type form (FIG. 3). Based on these observations, the monomeric form of IL-7 combined with W142H IL-7 mutation seems to allow an optimal conformation of the IL-7 molecule to promote IL-7 signaling into human T cells. Even with only one IL7, the molecule with W142H IL-7 mutation has an activation effect (pSTAT5) as good as a molecule with IL7 wt with two cytokines. This result is surprising in the context of an IL-7 variant having a lower affinity for its receptor than the wild type IL-7.

TABLE 3
Binding of anti PD1 IL7 wildtype or anti PD1
IL7 W142H mutant constructed with 1 or 2 IL7.

| | KD CD127 (M) |
|---|---|
| anti PD-1*2 IL7 wild type*2 | 8.7E−10 |
| anti PD-1*2 IL7 W142H*2 | 3.73E−8 |
| anti PD-1*2 IL7 W142H*1 | 4.52E−8 |

CD127 was immobilized to the sensor chip and anti PD-1 IL-7 bifunctional molecules were added at escalating doses to measure affinity.

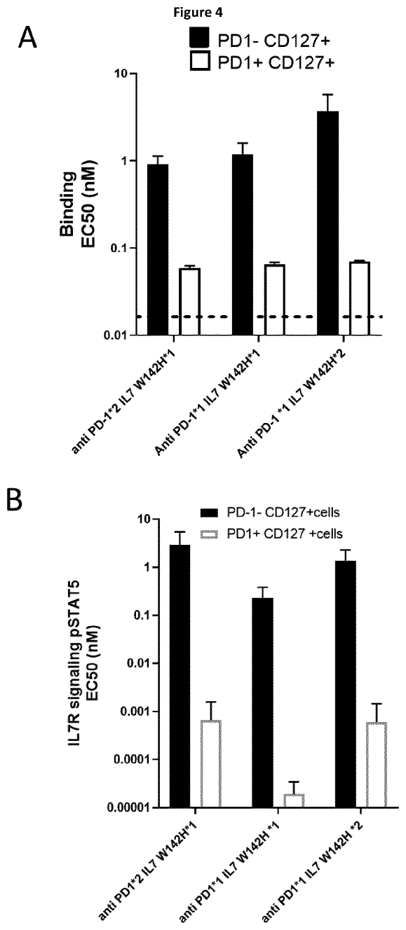

Finally, the specific cis-targeting and cis-activity of the different anti PD-1 IL-7 constructions were analyzed in a co-culture assay. U937 PD-1+ CD127+ cells were mixed with PD-1− CD127+ cells (ratio 1:1), then incubated with the different constructions at escalating doses. The binding and the IL-7R signaling (pSTAT5) was quantified by flow cytometry. EC50 (nM) of the binding and the pSTAT5 activation was determined for each construction and for each PD-1+ and PD-1− cell population (FIG. 4A and B). The inventors validated that a diversity of anti PD-1 IL-7 mutated molecules (anti PD-1*2 IL7 W142H*1, anti PD-1*1 IL7 W142H*1 anti PD-1*1 IL7 W142H*2) substantially preferentially bind IL-7R into PD-1+ cells, with a huge activation of IL7R signaling pSTAT5 into PD-1+ cells. Importantly, the construction PD-1*1 IL7 W142H*1 demonstrated the highest activity to stimulate the pSTAT5 signaling into PD-1+ cells compared to the other constructions (anti PD-1*2 IL7 W142H*1, and anti PD-1*1 IL7 W142H*2). These data suggest that the bifunctional molecule constructed with one anti PD-1 arm and one IL-7 has an optimal conformation and activity to allow the preferential activation of the IL-7R into PD-1+ activated T cells in the context of cancer.

### Example 2: Anti PD-1 IL-7 Molecules Constructed with 1 or 2 Arms of Anti PD-1 and 1 or 2 IL7 W142H Cytokines Have a Good Pharmacokinetic Profile In Vivo

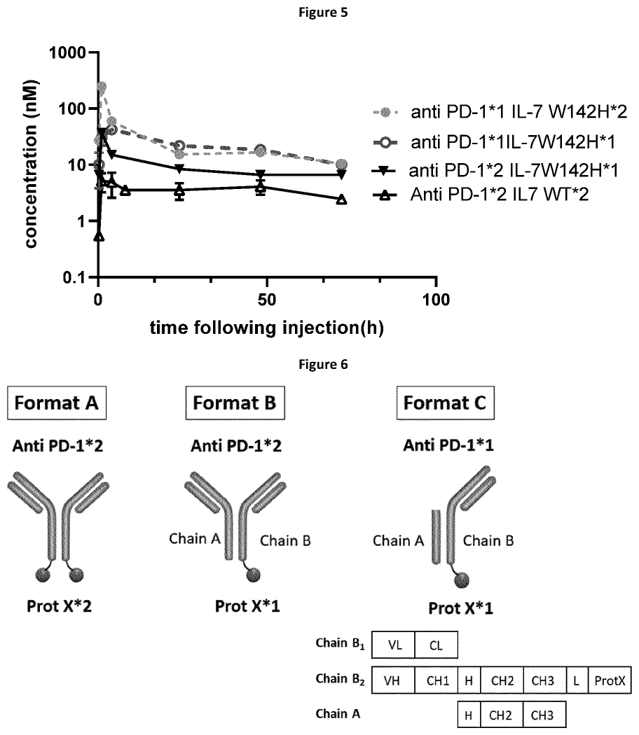

Pharmacokinetics study of the anti PD-1 IL-7 bifunctional molecules constructions 2, 3 and 4 such as described in FIG. 1 was assessed. Humanized PD1 KI Mice were intraperitoneally injected with one dose of anti PD-1 IL-7 molecules (34.4 nM/kg). Plasma drug concentration was analyzed by ELISA specific for human IgG (FIG. 5). Area under the curve was also calculated (see Table 4) and represents the total drug exposure across time for each construction. The anti PD-1*2IL-7 W142H*1, anti PD-1* 1 IL-7 W142H*1 and anti PD-1*1 IL-7 W142H*2 constructions demonstrated a very advantageously enhanced PK profile compared to the anti PD-1*2 IL7WT*1. A Cmax 2,8 to 19 fold higher was observed compared to the anti PD-1*1 IL7WT *2. Importantly, a high drug concentration (11-15 nM) which corresponds to a satisfying PK value in vivo, is maintained for at least 96 hours with the anti PD-1*1 IL7 W142H*1 anti PD-1*1 IL7 W142H*2 molecules whereas only 2 nM of anti PD-1*2 IL7WT*2 molecule is detected in the plasma. A residual drug concentration with the anti PD-1*2 IL-7 W142H*1 is 2,5-fold higher than the anti PD-1*2 IL7WT*2 concentration. Plasma drug exposure is often correlated with efficacy in vivo. Here, the inventors demonstrate that all anti PD-1 IL-7 W142H molecules constructed with one arm of anti PD-1 allows a long-term drug exposure following a single injection. Although the anti PD-1*2IL-7 W142H*1, anti PD-1* 1 IL-7 W142H*1 and anti PD-1*1 IL-7 W142H*2 demonstrated similar advantageous PK profile in vivo, the FIG. 4B demonstrate that the construction anti PD-1*1 IL-7 W142H*1 possess higher capacity to activate PD-1+ cells.

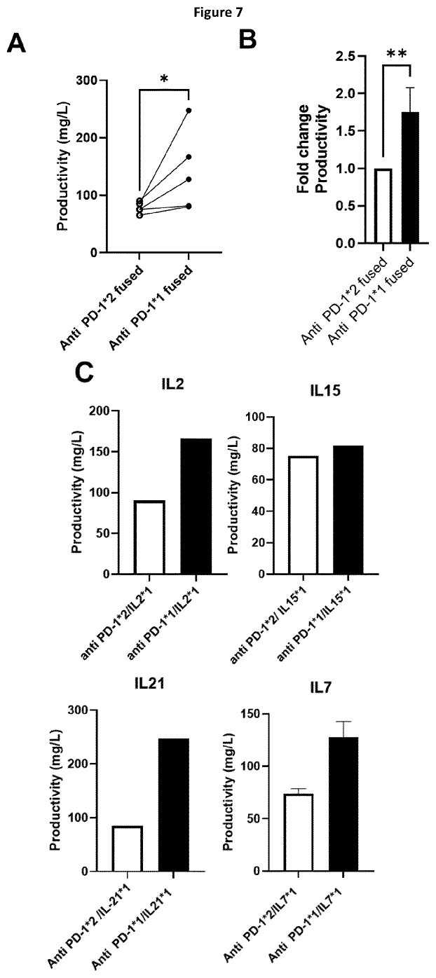

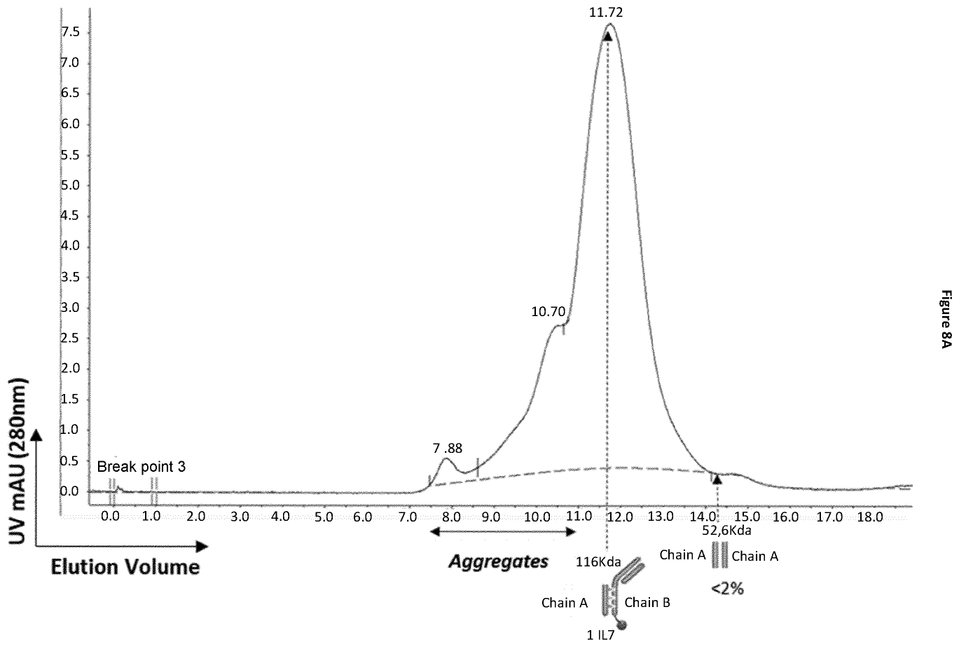

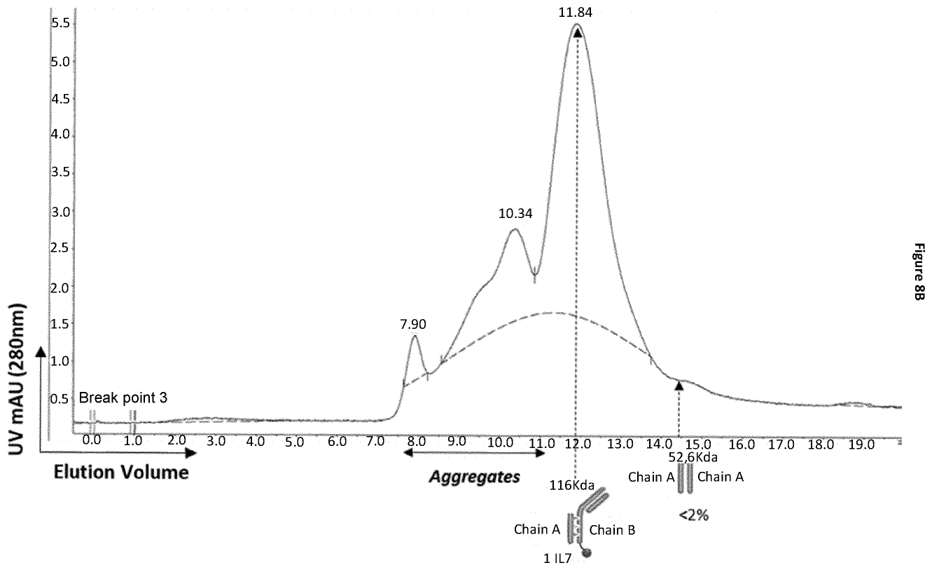

TABLE 4
Area under the curve determination from FIG. 7. AUC was
calculated from 0 to 96 hours following intraperitoneal
injection of one dose of anti PD-1 IL-7 (34 nM/kg).

| | AUC | Cmax (nM) |
|---|---|---|
| anti PD1*1 IL7W142H*1 | 1597 | 42.4 |
| antiPD-1*1 IL7W142H*2 | 2024 | 248.6 |

### Example 3: Description of the Constructions Used in the Examples 4 to 9

Different constructions of bifunctional antibodies were tested and compared. The FIG. 6 illustrates the different formats: (1) Format A (anti PD-1*2/prot X*2), (2) Format B (Anti PD-1*2/Prot X*1) (3) Format C (anti PD-1*1/protX*1 fused to the heavy chain). For the Format C, the Fc domain contains the CH1 CH2 and Hinge parts. All constructions were engineered with an IgG1 N298A isotype and amino acid sequences were mutated in the Fc portion to create a knob on the CH2 and CH3 of the Heavy chains A and a hole on the CH2 and CH3 of the Heavy chains B. All constructions comprise an GGGGSGGGGSGGGGS linker (SEQ ID NO: 70) between the Fc domain and the X protein fused.

### Example 4. Bifunctional Antibodies Constructed with One Anti PD-1 Valency and One Fused Protein X Demonstrated Higher Productivity by Mammalian Cells Compared to Bifunctional Antibody Constructed with Two Anti PD-1 Valencies and One Fusion Used Protein X

The productivity of the format B and format C of the bifunctional antibodies by mammalian cells was assessed and compared. Full Heavy chain with a Fc fused to cytokine (IL,7, IL-2, IL-15 or IL-21) were transiently co-transfected with the light chains into CHO suspension cells. Quantity of antibody obtained after production and purification was quantified using a sandwich ELISA (immobilized donkey anti human Fc antibody for detection and revelation with a mouse anti human kappa+a peroxidase conjugated goat anti mouse antibody). Concentration was determined with human IvIgG standard. Productivity was calculated as the quantity of purified antibody per liter of collected culture supernatant.

Results: Bifunctional antibody, anti PD-1*2/protX*1 (Format B) and antibody PD-1*1 ProtX*1 (Format C) were produced in CHO mammalian and the results presented in FIG. 7. In a surprising manner, the anti PD-1*1/prot X*1 construction (Format C) has a significant better productive yield (mg/L) than the anti PD-1*2/prot X*1 (Format B) (FIG. 7A). A significant 1.7-fold (+/−0.7; n=5) higher productivity was obtained (FIG. 7B) for all tested fused protein X, i.e., cytokines (IL7, IL2, IL21 and IL-15). Those results indicate that the anti PD-1*1/prot X*1 (Format C) presents a very good manufacturability which is very important for the next steps of the clinical development and therapeutic applications.

Especially, for bifunctional antibody that comprises two different arms, one major problem is the mispairing of the chains and the incorrect association of the Chain A (Knob chain) with the chain B (Hole chain). Indeed, undesired homodimer formation (Chain A+Chain A or Chain B+Chain B) generally occurs. This would normally lead to a low yield and purity of heterodimeric bifunctional antibody (Formats B and C) compared to the production of the homodimeric bifunctional antibody (Format A), which is a significant disadvantage. A key challenge remains how to produce uniform bifunctional antibody with high quality and limited or negligible side products and impurities.

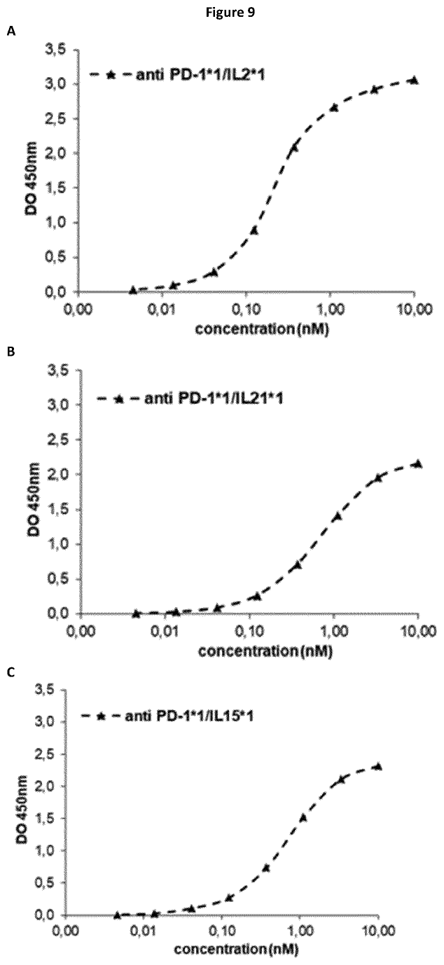

However, the inventors demonstrated that, by using the optimized strategy design of the Format C, a higher production of the bifunctional antibody surprisingly induces compared to the homodimeric format B (FIG. 9A). Moreover, the productivity yield of the anti PD-1*1 ProtX*1 is in a similar range than an anti PD-1 alone (anti PD-1*1 or anti PD-1*2), the productivity of which is equal to 45 mg/L (n=5) in similar conditions of production.

The other major issue of the production of a heterodimeric antibody is the purity. Although the knob-into hole strategy favors heterodimeric production (Chain A+Chain B) and reduces the homodimer chain A or the homodimer chain B production, this strategy is not 100% effective and an additional purification is required to isolate the heterodimer construction (Wang et al, 2019, Antibodies, 8, 43).

However, the inventors observed that a high yield of heterodimer is obtained after production of the anti PD-1*1/Prot X*1 Format C. FIG. 8 shows the size exclusion chromatography of the anti PD-1*1/IL-7wt*1 (FIG. 8A) and the anti PD-1*1/IL-7v*1 (FIG. 8B) after protein A purification. One major peak corresponding to the heterodimeric form chain A and the chain B, whereas the homodimeric form Fc/Fc (Chain A+chain A) was not detected and the homodimeric form (Chain A+chain A) was very minor (less than 2%). These data suggest that the present invention anti PD-1*1/protX*1 is optimized for productivity and prevents the mispairing. In comparison to the prior art, a yield of heterodimer around 70 to 75% of heterodimer is obtained with other bispecific antibody backbones using the same KIHs-s strategy.

To obtain such purity, the inventors optimized the design of the molecule. In fact, they observed that this high purity was only obtained if the chain A is the Fc domain and the chain B is the anti PD-1*1 IL-7*1. The co-transfection of the VL+the sole chain B (anti PD-1*1/IL-7v*1) containing the hole mutation into CHO mammalian cells does not induce the production of homodimer of chain B (0 mg/L). At the contrary, if the anti PD-1*1/IL-7v*1 is the chain A comprising the knob mutation, a high production of homodimer Chain A is obtained (88 mg/L) after cotransfection of the VL+the sole chain A (anti PD-1*1/IL-7v*1). According to these data, the inventors selected to design the molecule with the Fc as chain A and the anti PD-1*1/protX*1 as chain B to avoid the production of homodimer of Chain A.

Altogether, these data shows that the format C according to the present invention Anti PD-1*1/ProtX*1 with a chain A (Fc domain with the knob mutation) and chain B (Anti PD-1*1/IL-7* with the hole mutation) is the best construction for high productivity and purity of the product. This facilitates the development as therapeutic agent for large scale productivity.

### Example 5: Competitive Assays to Measure the Activity of the Anti-PD1 Bifunctional Antibody and Their Capacity to Antagonize PD-1/PD-L1 Interaction

The binding of bifunctional antibody to bind to human PD-1 receptor and to antagonize PD-L1/PD-1 interaction was measured by ELISA assays. Recombinant hPD1 (Sino Biologicals, Beijing, China; reference 10377-H08H) was immobilized on plastic at 0.5 μg/ml in carbonate buffer (pH 9.2) and purified antibody was added to measure binding. After incubation and washing, peroxidase-labeled donkey anti-human IgG (Jackson Immunoresearch; USA; reference 709-035-149) was added and revealed by conventional methods.

To measure antagonist activity of the bifunctional antibody, a competitive ELISA assay was performed by PD-1:PD-L1 Inhibitor Screening ELISA Assay Pair (AcroBiosystems; USA; reference EP-101). In this assay, recombinant hPDL1 was immobilized on plastic at 2 μg/ml in PBS pH 7.4 buffer. Purified antibodies (at different concentrations) were mixed with 0.66 μg/ml final (fix concentration) of biotinylated Human PD1 (AcroBiosystems; USA; reference EP-101) to measure competitive binding for 2 h at 37° C. After incubation and washing, peroxidase-labeled streptavidin (Vector laboratoring; USA; reference SA-5004) was added to detect the c binding and revealed by conventional methods.

Results: FIG. 9 and table 5 show that all bifunctional anti PD-1*1/protX*1 molecules demonstrate an efficient PD-1 binding. These data show that all molecules with every type of fused proteins, even with one anti PD-1 valency, conserve a good binding to PD-1 antigen. Moreover, the antagonist activity is also preserved as shown in Table 6.

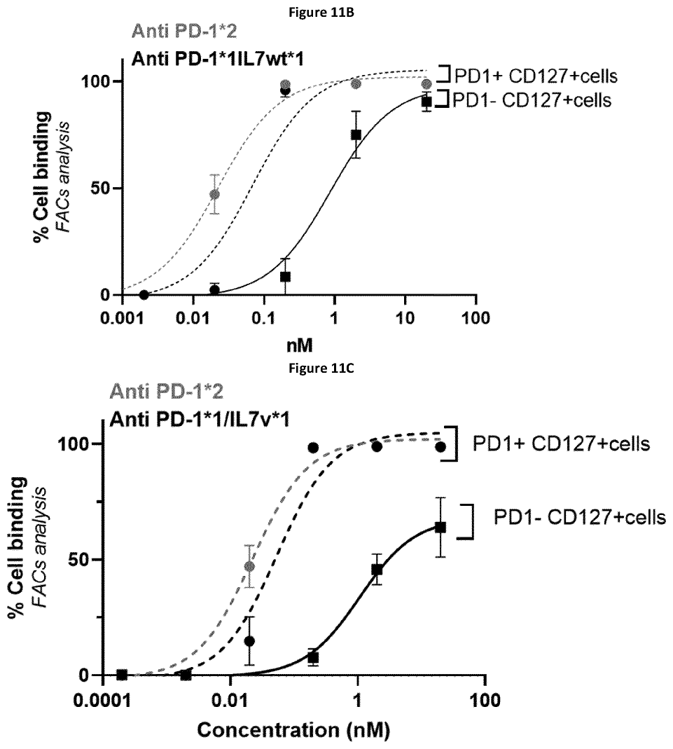

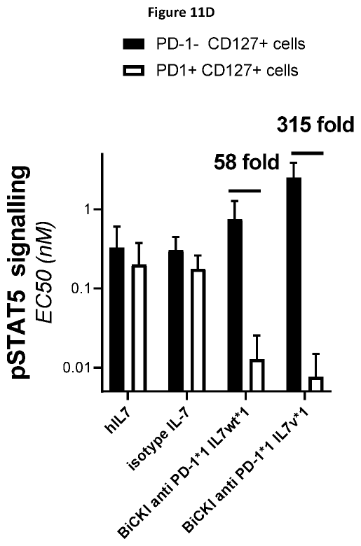

TABLE 5
EC50 (nM) of the PD-1 Binding from the FIG. 11.

| PD-1 binding | EC50 (nM) |
|---|---|
| anti PD-1*1/IL2*1 | 0.20 |
| anti PD-1*1/IL21*1 | 0.58 |
| anti PD-1*1/IL15*1 | 0.51 |

TABLE 6
EC50 (nM) Competition PD-1/PD-L1 ELISA assay.

| PD-1/PD-L1 antagonist | IC50 (nM) |
|---|---|
| anti PD-1*1/IL2*1 | 2.5 |
| anti PD-1*1/IL21*1 | 20.3 |
| anti PD-1*1/IL15*1 | 7.1 |

### Example 6: Anti PD-1/Cytokine Bifunctional Antibody Can Activate pSTAT5 Signaling into Primary Human T Cells

The inventor next assessed the biological activity of the protein fused to the anti PD-1 antibody and tested the capacity of all anti PD-1/cytokine bifunctional molecules to activated primary T cells. For this purpose, human peripheral blood T cells were treated 15 minutes at 37° C. with different concentrations of the anti PD-1*1/IL-7wt*1, the anti PD-1*1/IL-7v*1, the anti PD-1*1/IL-15*1, the anti PD-1*1/IL-21*1 constructions. After incubation, cells were fixed, permeabilized and stainined with an anti pSTAT5 antibody.

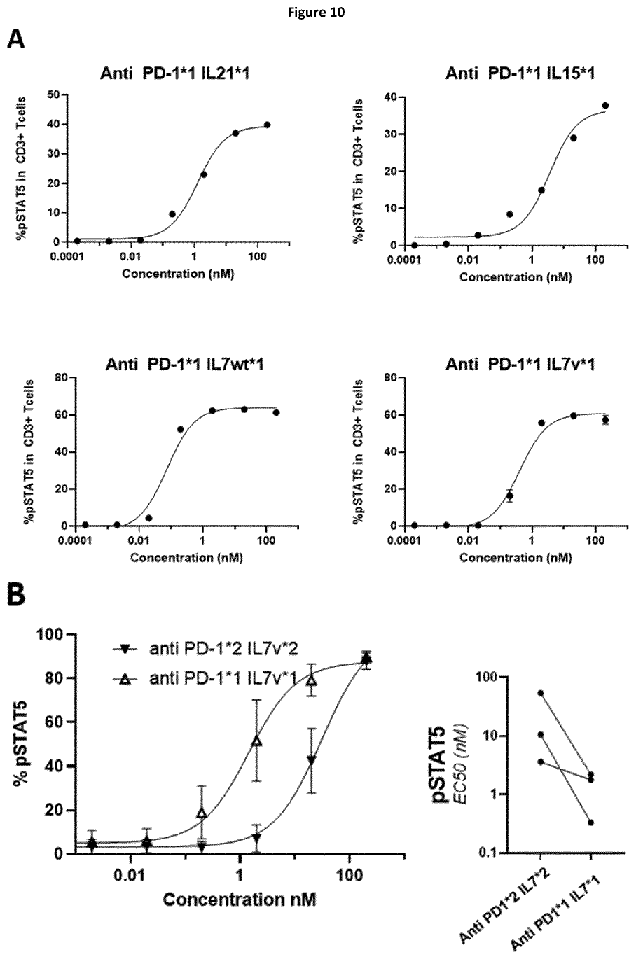

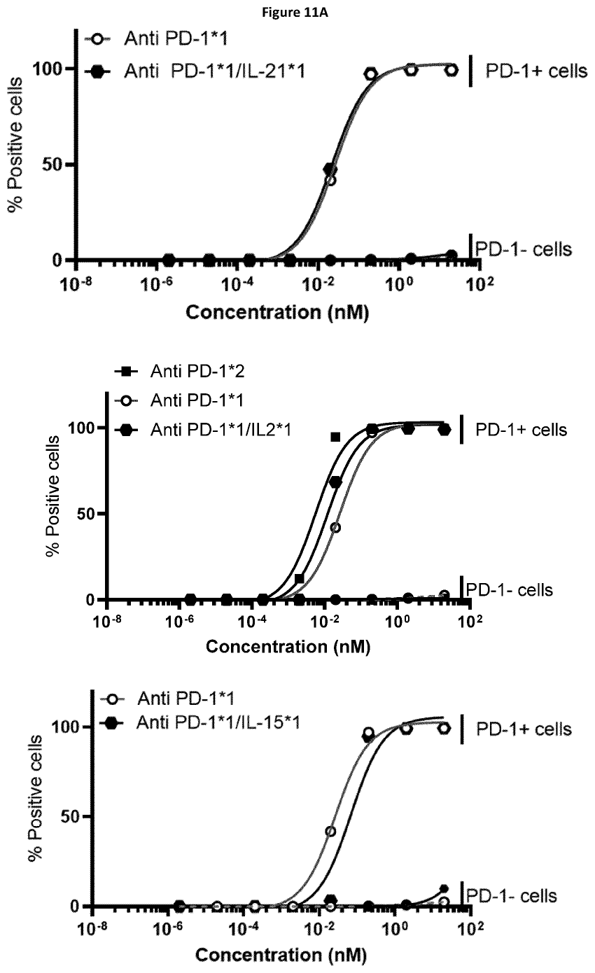

Results: FIG. 10A shows that the anti PD-1*1/IL-7wt*1, the anti PD-1*1/IL-7v*1, the anti PD-1*1/IL-15*1 and the the anti PD-1*1/IL-21*1 efficiently induce pSTAT5 signalling into primary T cells (CD3+ T cells), suggesting that the cytokine fused to the Fc domain of anti-PD-1 molecules of Format C conserves its capacity to stimulate human T cells. The inventors next compared the efficiency of the anti PD-1/IL-7 Format A (Anti PD-1*2/IL7v*2) versus the anti PD-1*1/IL-7v*1 Format C to activate pSTAT5 signaling. Since the construction anti PD-1*1/IL-7v*1 comprises only one IL-7v cytokine, a lower pSTAT5 activation was expected for this molecule in comparison to Format A. However, as shown on FIG. 10B, a higher pSTAT5 activation was surprisingly observed with the anti PD-1*1/IL-7v*1 (Format C) versus anti PD-1*2/IL-7v*2 construction (Format A), suggesting that the present invention anti PD-1/ProtX*1 construction (Format C) allows an optimal conformation of the IL-7 molecule to promote activation of IL-7 signaling into primary T cells.

### Example 7: The Anti PD-1 Bifunctional Molecules Allow Preferential Binding on PD-1+ Over PD-1− Cells and the Anti PD-1/IL7 Molecules Allows a Synergistic Activation of TCR Signaling into PD-1+ T Cells

The inventors assessed the capacity of the anti PD-1 bifunctional molecules to target PD-1+ T cells and to allow a preferential delivery and a cis-binding of the cytokine or protein fused to PD-1 + cells. U937 PD-1− cells and U937 PD1+ CD127+ cells were cocultured (ratio 1:1) and incubated with anti PD1/ProtX molecules at escalating doses. The binding and the IL-7R signaling (pSTAT5) was quantified by flow cytometry. EC50 (nM) of the binding and the pSTAT5 activation was determined for each construction and for each PD-1+ and PD-1− cell populations. In parallel, the binding of the bifunctional antibody was detected with an anti IgG-PE (Biolegend, clone HP6017) and analyzed by flow cytometry.

Results: FIG. 11A shows that all anti PD-1/protX molecules, namely the anti PD-1*1/IL-2*1, the Anti PD-1*1/IL-21*1, the anti PD-1*1/IL15*1 molecules, bind with high efficacy to PD-1+ cells over PD-1− cells, with similar efficacy compared to an anti PD-1*1 and anti PD-1*2 antibody, confirming the high binding efficacy of the molecule constructed with one anti PD-1 valency versus 2 anti PD-1 valency. FIG. 11B and 11C show the binding of the anti PD-1*1/IL-7wt*1 and the anti PD-1*1/IL7v*1 molecules on cells expressing CD127+ only or coexpressing CD127 and PD-1 receptors. Data show that both molecules preferentially bind to PD-1+CD127+ cells over PD-1−CD127+ cells, with comparable efficacy to an anti PD-1 alone (Anti PD-1*2). In parallel, the activation of PSTAT5 signaling into PD-1+ cells versus PD-1− cells was also assessed as detailed on FIG. 11D. A strong activation of IL7R signaling pSTAT5 into PD-1+CD127+ cells versus PD-1−CD127+ cells after treatment with the anti PD-1*1/IL-7wt or IL7v*1 antibody (58 to 315 fold higher activation) whereas the isotype/IL7 antibody has similar efficacy in PD-1+ and PD-1− cells confirming that the anti PD-1 domain of the anti PD-1*1/IL-7*1 molecule allows the preferential binding of IL-7 on PD-1+ cells, i.e., targeting of the drug and activation on the same cell. This aspect has an interest for the biological activity of the drug in vivo, as the anti PD-1 IL-7 will concentrate the IL-7 or other molecules fused in the bifunctional molecule on PD-1+ tumor specific T cells into the tumor microenvironment over PD-1 negative naïve T cells. Altogether, this data shows that only one arms of anti PD-1 is sufficient to allow the selective delivery of the fused cytokine on PD-1+ cells.

Next, the inventors assessed the biological impact of the cis-targeting of the anti PD-1 bifunctional molecule on PD-1+ T cells. A Promega PD-1/PD-L1 kit (Reference J1250) assay was used. Briefly, two cell lines are used (1) Effector T cells (Jurkat stably expressing PD-1, NFAT-induced luciferase) and (2) activating target cells (CHO K1 cells stably expressing PD-L1 and surface protein designed to stimulate cognate TCRs in an antigen-independent manner). When cells are cocultured, PD-L1 /PD-1 interaction inhibits TCR mediated activation thereby blocking NFAT activation and luciferase activity. The addition of an anti-PD-1 antibody blocks the PD-1 mediated inhibitory signal and restores TCR mediated signaling leading to NFAT activation and luciferase synthesis and emission of bioluminescence signal.

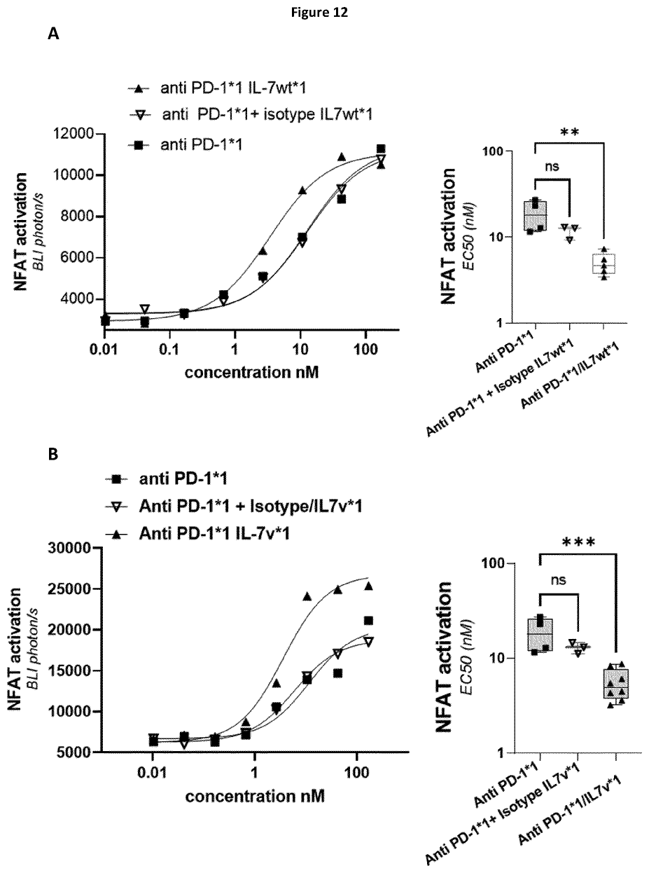

Results of the bioassay are presented on FIG. 12A and show that the bifunctional anti PD-1*1/IL7wt*1 molecule is better than an anti-PD1*1 or an anti-PD1*1+ non targeted isotype-IL7 (as separate compounds) to activate TCR mediated signaling (NFAT), demonstrating a synergistic effect of the bifunctional molecule on PD1+ T cells. The anti PD-1*1/IL-7v*1 molecule including an IL-7 mutant also showed a significant synergistic effect to reactivate NFAT signaling onto T cells (FIG. 12B). These data show that the fusion of the one IL-7 cytokine to one anti PD-1*1 advantageously induce a higher activation of TCR signaling whereas combinatory strategy as two sperate compounds do not induce such efficacy.

### Example 8. Bifunctional Molecules with One Anti PD-1 Valency Demonstrated a Better Pharmacokinetics In Vivo

Pharmacokinetics and Pharmacodynamics of the product were assessed in mice following a single injection. C57bl6JRj mice (female 6-9 weeks) were intravenously or intraperitoneally injected with a single dose (34 nmol/kg) of anti PD-1 or bifunctional antibodies. Plasma drug concentration was determined by ELISA using an immobilized anti-human light chain antibody (clone NaM76-5F3), then serum-containing antibodies were added. Detection was performed with a peroxidase-labeled donkey anti-human IgG (Jackson Immunoresearch; USA; reference 709-035-149) was added and revealed by conventional methods. Area under the Curve corresponding to the drug exposure was calculated for each construction.

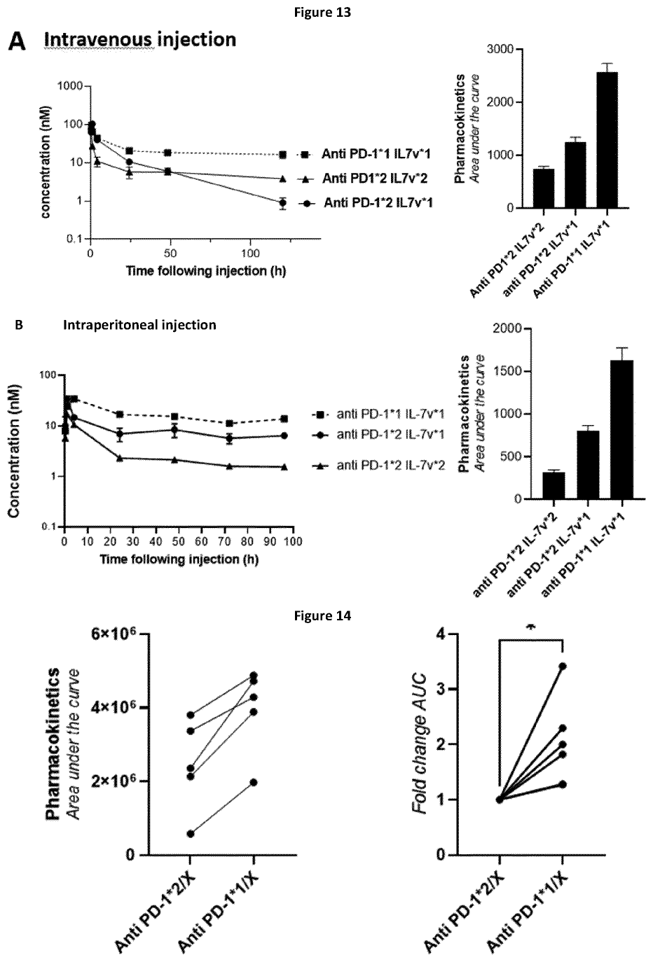

Results: The inventors first compared the pharmacokinetics of all different bifunctional formats described in the FIG. 6 (Formats A, B and C). FIG. 13 shows the pharmacokinetics profile of the anti PD-1/IL-7v constructed with 1 or two IL-7v cytokines and 1 or 2 anti PD-1 valencies after a single intravenous (FIG. 13A) or intraperitoneal (FIG. 13B) injection. The anti PD-1*1/IL-7v*1 (Format C) demonstrated the best pharmacokinetics profile in comparison to the anti PD-1*2/IL-7v*2 (Format A) and the anti PD-1*2/IL-7v*1 (Format B). Both intravenous and intraperitoneal injections demonstrated that the anti PD-1*1/IL-7*1 (Format C) is an optimal construction to enhance pharmacokinetic of the bifunctional molecule.

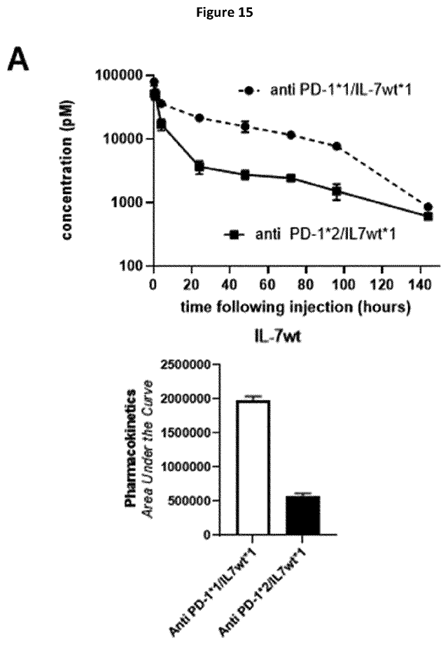

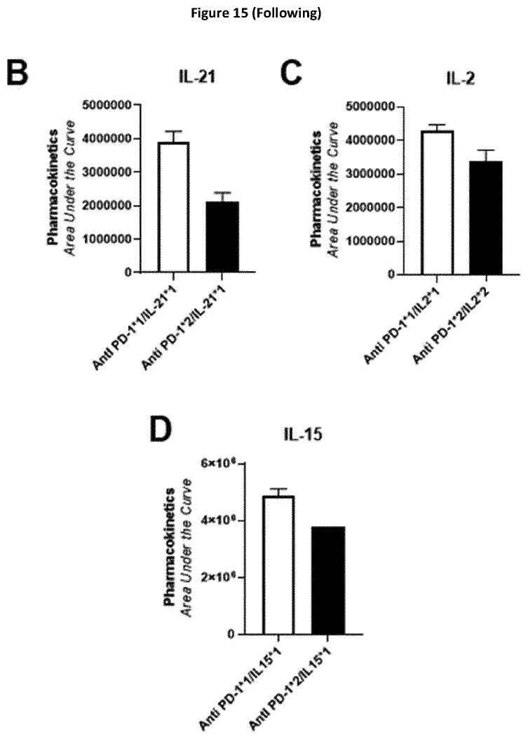

Next, the inventors tested whether bifunctional molecules with 1 anti PD-1 arm (Anti PD-1*1) and other cytokines (IL-7wt, IL-21, IL-2, IL-15) demonstrated better in vivo pharmacokinetic profile compared to the same bifunctional molecule constructed with 2 anti PD-1 arms. FIG. 14 shows the pool of all proteins tested (AUC and fold change AUC) and FIG. 15 shows the result for individual constructions. Data show that all bifunctional molecules anti PD-1*1/protX (Format C) demonstrated a significant better Pharmacokinetics profile compared to the corresponding bifunctional molecules anti-PD-1*2/ProtX*1(Format B).

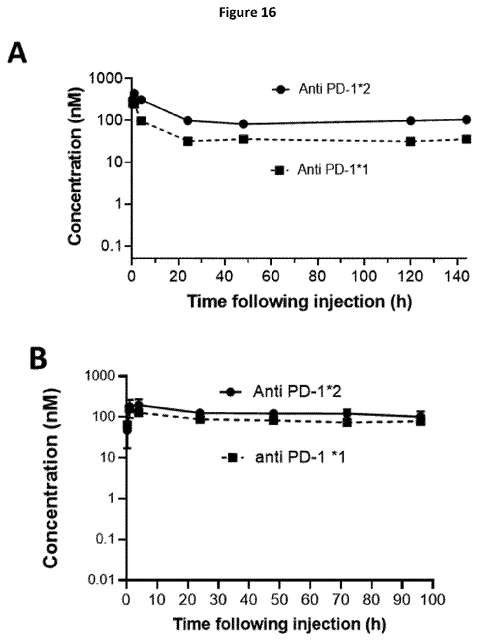

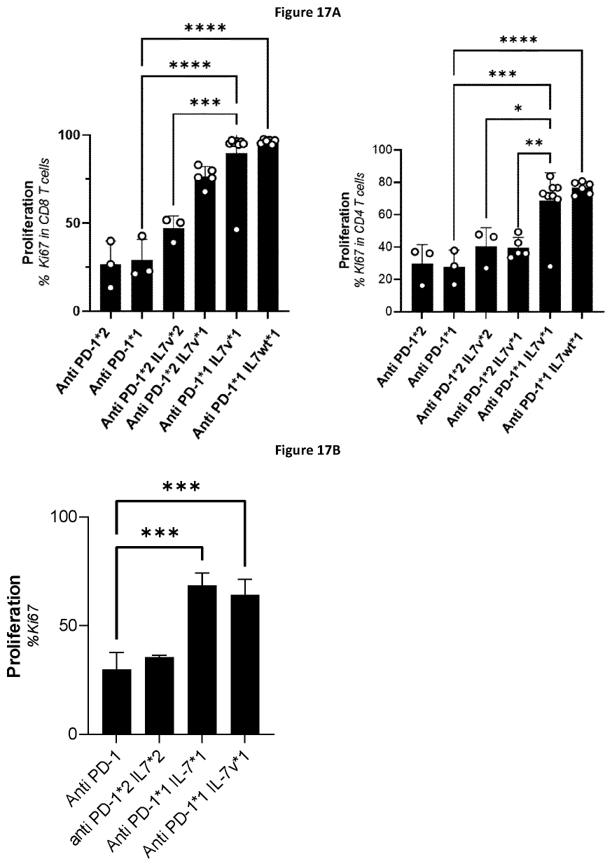

In another experiments, pharmacokinetics of anti PD-1*2 or anti PD-1*1 antibody alone was also assessed to understand whether the anti PD-1 construction alone allows a better pharmacokinetics profile or whether this observation is only applicable to bifunctional molecules. FIG. 16 shows that both anti PD-1*2 and anti PD-1*1 have similar profile after intravenous (FIG. 16A) or intraperitoneal (FIG. 16B) injection, suggesting that, surprisingly, the anti PD-1*1 construction induces a better pharmacokinetics profile only for bifunctional molecules.

The poor pharmacokinetics profile is a well-known challenge for bifunctional antibodies. Bifunctional antibodies are rapidly eliminated and present a short half-life in vivo limiting their use in clinic. A good drug concentration (20 and 100 nM) which corresponds to a satisfying PK value in vivo, is maintained for at least 48-72 hours with the anti PD-1*1/ProtX*1 constructions whereas only 2 nM of anti PD-1*2 IL7*2 molecule is detected in the plasma. The format C anti PD-1*1/protX*1 of the present invention allows to improve pharmacokinetics profile in vivo, with longer term exposure compared to other format of bifunctional antibodies (format B and C).

### Example 9 The Anti PD-*1/IL-7*1 Bifunctional Molecule Promotes In Vivo Proliferation of T Cells and Induces Significant Anti-Tumor Efficacy Compared to the Anti PD-1*2/ IL*7*2 or Anti PD-1*2/ IL*7*1 Constructs

In vivo proliferation of T cells was assessed after a single dose of bifunctional molecules (34 nM/kg) intraperitoneally injected into bearing a subcutaneous MC38 tumor. On Day 4 following treatment, blood and tumor was collected, and T cells were stained with an anti Ki67 antibody to quantify proliferation by flow cytometry.

In vivo efficacy was assessed in 2 different orthotopic syngeneic models, an hepatocarcinoma model and a mesothelioma orthotopic models. Immunocompetent mice genetically modified to express human PD-1 (exon 2) were used for these experiments. For mesothelioma model, AK7 mesothelial cells were intraperitoneally injected (3*10⁶ cell/mouse). For the Hepa 1.6 model, 2.5*10⁶ cells were injected in the portal vein. In the experiment 1, mice treated with PBS, anti PD-1 control (anti PD-1*2), anti PD-1*1/IL*7v*1 at similar drug exposure concentrations. In the experiment 2, mice were treated with PBS, anti PD-1 control (anti PD-1*2), anti PD-1*1/IL*7wt*1at similar drug exposure concentration. AK7 cells stably express luciferase allowing the quantification of tumor burden in vivo after D-luciferin injection Data were analyzed in photon per second per cm2 per steradian and represent the mean of the dorsal and ventral signal.

Results: FIG. 17A shows that anti PD-1*1/IL7wt*1 or IL7v*1 bifunctional molecules (Format C) promote significant proliferation of CD4 and CD8 T cells to higher extent than anti-PD-1 antibody (anti PD-1*1 or anti PD-1*2). A significant superior CD4 T cells of these 2 constructions was also observed compared to the anti PD-1*2/IL7*2 (Format A) and the anti PD-1*2/IL7*1 (Format B) constructions. Similarly, a higher proliferation, was observed after treatment with anti PD-1*1/IL7wt*1 or anti PD-1*1/IL-7v*1 compared to the anti PD-1*2/IL7*2 (Format A) and the anti PD-1*2/IL7*1 (Format B) constructions. These data corroborate with the efficiency of the different constructions to activate pSTAT5 signaling into T cells (FIG. 10A) where the anti PD-1*1/IL7*1 constructions induced a higher pSTAT5 signaling compared to the anti PD-1*2/IL7*2 constructions.

Interestingly, the inventors observed that the anti PD-1*1/IL7wt*1 or IL7v*1 significantly induces proliferation of Stem-like effector memory CD8 T cells into the tumor, to significant higher extent to the anti PD-1*2 IL-7*2 and the anti PD-1*2 molecules (FIG. 17B). The capacity of the anti PD-1*1 IL-7*1 to boost the TCF1+stem like CD8 T cell population is particularly interesting since are critical for immune control of cancer. These cells are capable of se renewal to generate a pool of tumor specific T cells with high effector functions.

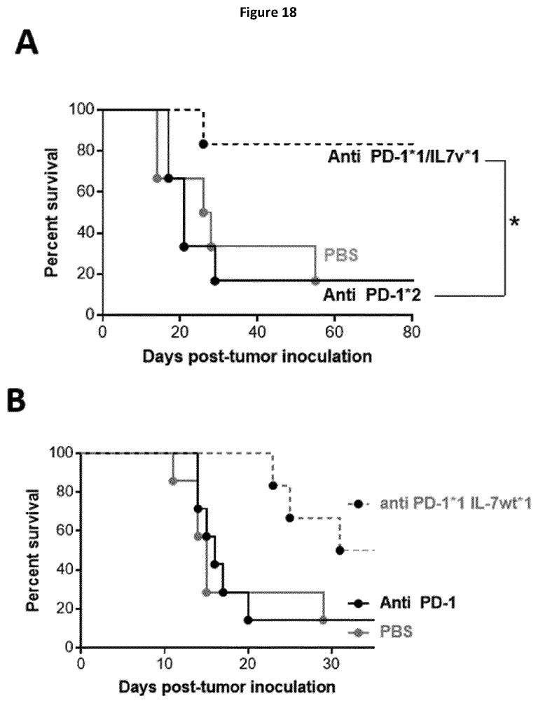

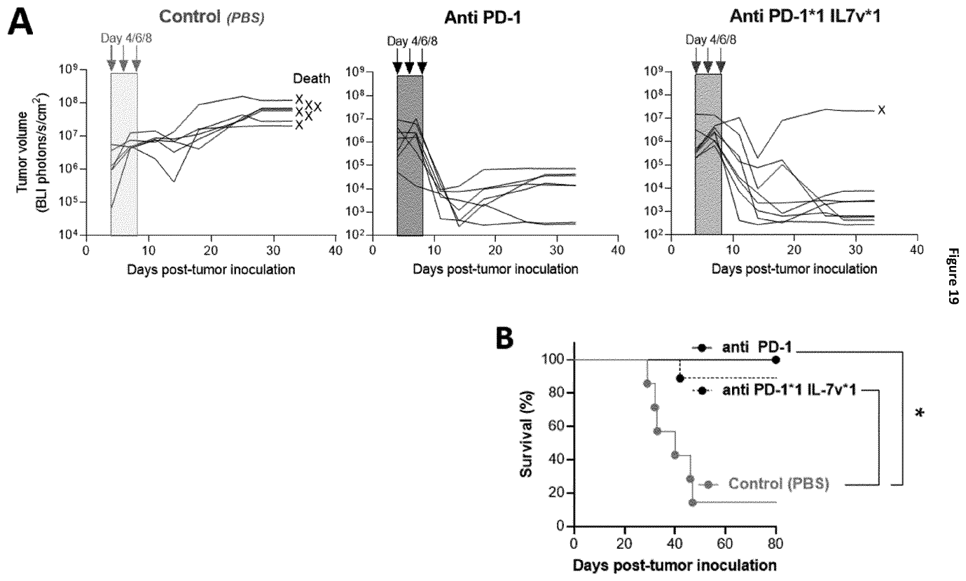

FIGS. 18A and 18B show the in vivo efficacy of the anti PD-1*1 IL-7wt*1 and the anti PD-1*1 IL-7v*1 in the orthotopic hepatocarcinoma model. In two separate experiments, the Anti PD-1*1/IL*7*1 (both wt and variant) (Format C) demonstrated a significant superior efficacy compared to the anti PD-1*2 antibody. 85% of Complete tumor eradication (complete response) were obtained after treatment with anti PD-1*1/IL7v*whereas only 16% of mice treated with anti PD-1*2 developed a complete tumor response.

In comparison, the inventors also tested the construction anti PD-1*2/IL7v*2, and low anti-tumor efficacy was observed with the anti PD-1*2/IL-7*2 construction in the same hepatocarcinoma model, underlying the superior in vivo activity of the anti PD-1*1/IL7*1 construction versus the anti PD-1*2/IL-7*2.

In the Mesothelioma orthotopic model (FIGS. 19A and B), anti PD-1*1/IL7v*1 demonstrated high anti-tumor efficacy with >85% of complete response similar to anti PD-1*2 antibody treatment. These data show that, in an anti PD-1 sensitive model, the anti PD-1*1/IL7v*1 is highly effective, suggesting that, even if the Formats C comprises only one anti PD-1 arm (Anti PD-1*1), the drug shows similar efficacy to an anti PD-1 with 2 valencies.

Altogether, these data underline that the design of the bifunctional antibody is crucial to obtain an anti-tumor efficacy in vivo. The fusion of one cytokine or protein to anti PD-1*1 (Format C) shows the best anti-tumor efficacy and T cell in vivo proliferation whereas bifunctional molecules constructed with 2 anti PD-1 arms and one or 2 cytokines or proteins did not induce efficient proliferation of T cells in vivo nor anti-tumor efficacy.

### Example 10: Anti PD-1*1 IL-7v*1 Construction Abrogates Suppressive Functions of Treg In Vitro to Higher Extent than IL-7 Cytokine and Anti PD-1*1 IL7WT*1 Bifunctional Antibody

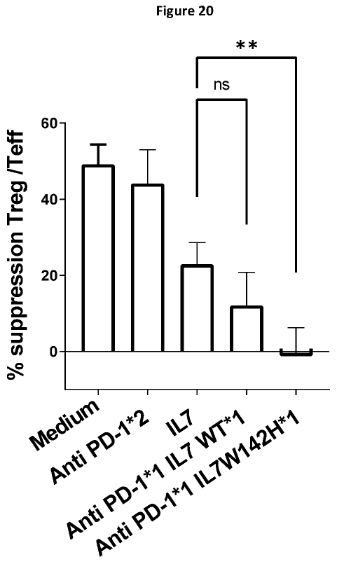

Although anti-PD1 therapy stimulates T cell effector functions, immunosuppressive molecules (TGFB, IDO, IL-10 . . . ) and regulatory cells (Treg, MDSCs, M2 macrophages) create a hostile microenvironment that limits full potential of the therapy. Treg cells express a low level of IL-7R (CD127), but they are still able to stimulate pSTAT5 following IL-7 treatment and IL7 is known to disarm Treg suppressive functions (Allgäuer A, et al. J. Immunol. 2015, 195, 31393148; Liu W, et al. J Exp Med. 2006, 203, 1701-1711; Seddiki N, et al. J exp Med 2006, 203, 1693-1700; Codarri L, et al. J exp Med 2007, 204, 1533-1541; Heninger A K , et al. J immunol 2012, 189, 5649-5658). To assess efficacy of anti PD-1/IL7 constructions to disarm Treg function compared to IL-7, a suppressive assay by coculturing Treg and T effector cells was performed. The inventors observed FIG. 20 that IL-7 or anti-PD1-IL7 treatments block Treg mediated inhibitory effect allowing proliferation of Teff cells even in the presence of Treg cells. The anti-PD1 antibody is not able to inhibit Treg suppressive activity on T effector cells.

Surprisingly, Anti PD-1*1 IL7 W142H*1 demonstrated the highest efficacy to suppress Treg functions compared to IL-7 cytokine (**p<0.05) but also compared to anti PD-1*1 IL7WT*1 construction. These data highlight the advantage of using Anti PD-1*IL7W142H*1 construction over naked IL-7 cytokine or non-mutated version of anti PD-1*1 IL7*1 bifunctional antibody. It was unexpected that the monovalent variant impact both Treg abrogation and simultaneously strong T cell proliferation, a double effect since the selected IL7 variant W142H presents lower affinity to IL7R compared to IL-7 cytokine wild type form.

Methods: Assessment of suppressive activity of Treg in vitro on CD8 effector T cell proliferation. CD8+ effector T cells and autologous CD4+ CD25high CD127low Treg were sorted from peripheral blood of healthy donor, stained with cell proliferation dye (CPDe450 for CD8+ T cells). Treg/CD8+Teff were then co-cultured at ratio 1:1 on OKT3 coated plate (2 μg/mL) for 5 days and proliferation of Teff cells was quantified by Flow cytometry with the loss of CPD marker.

### Example 11: Anti PD-1*1 IL-7v*1 Demonstrated Superior Efficacy In Vivo Compared to Anti PD-1*1 IL7WT*1 Construction in 2 Different Tumor Models

In vivo efficacy was assessed in 2 different orthotopic syngeneic models, an hepatocarcinoma model and a mesothelioma orthotopic models. Immunocompetent mice genetically modified to express human PD-1 (exon 2) were used for these experiments. For mesothelioma model, AK7 mesothelial cells were intraperitoneally injected (3*10⁶ cell/mouse). For the Hepa 1.6 model, 2.5*10⁶ cells were injected in the portal vein. In the experiment 1, mice treated with PBS, anti PD-1 control (anti PD-1*2), anti PD-1*1/IL7v*1 (Anti PD-1*1 IL7W142H*1) or anti PD-1*1 IL7wt*1 at similar drug exposure concentrations. AK7 cells and Hepa1.6 stably express luciferase allowing the quantification of tumor burden in vivo after D-luciferin injection. Data were analyzed in photon per second per cm2 per steradian and represent the mean of the dorsal and ventral signal.

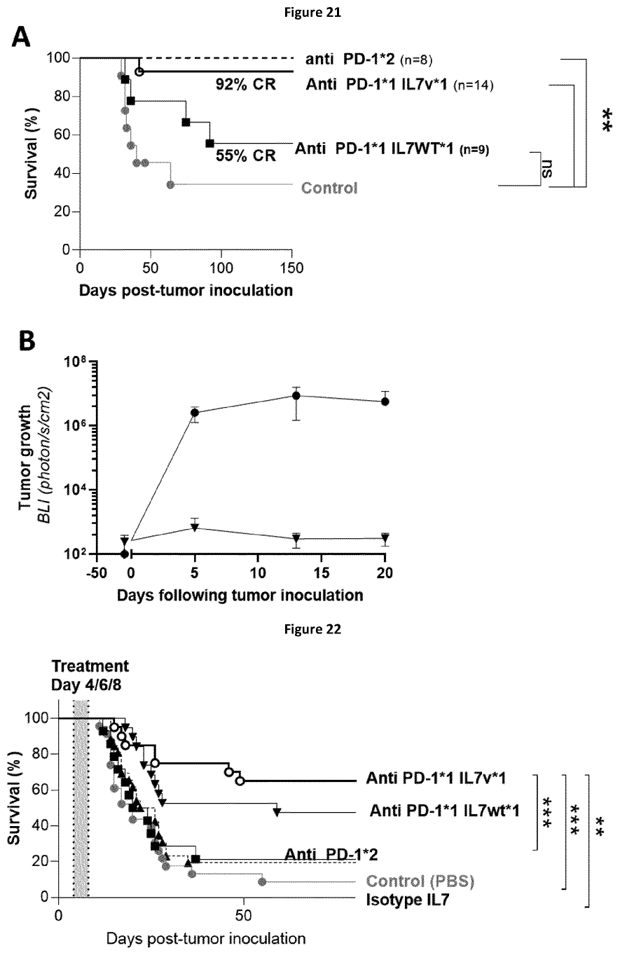

AK7 intraperitoneal model is highly sensitive to PD-1 antibody treatment associated with high CD4+ and CD8+ T cell infiltration expressing PD-1 was observed into the tumor microenvironment allowing a good response of anti PD-1 antibody as demonstrated in FIG. 19. In the same experiment, efficacy of anti PD-1*1 IL7v*1 was compared to the efficacy of its wild-type IL7 homolog construction (Anti PD-1*1 IL7wt*1). Anti PD-1*1 IL7v*1 construction induced 92% of complete response (n=1 death/14 mice) and superior efficacy compared to anti PD-1*1 IL7wt*1 construction that induced a moderate anti-tumor efficacy (62% of complete response) (FIG. 21A). Tumor bioluminescence analysis confirmed that anti PD-1*1 IL7v*1 induced tumor clearance within 11 to 18 days following treatment, whereas in the anti PD-1*1 IL7wt*1 group, tumors shrank after treatment then eventually relapse (data not shown) indicating that the efficacy may be transient with IL-7 wild type construction compared to bifunctional antibody constructed with low affinity IL-7 (IL7W142H).

To assess memory response induced by anti PD-1*1 IL7v*1 treatment, all cured mice treated with anti PD-1*1 IL7v*1 were rechallenged with second injection of AK7 mesothelioma cells. As shown in FIG. 21B, no tumor bioluminescence was detected after tumor rechallenge whereas a high bioluminescence signal was detected in Naïve challenged mice at multiple time points. These data demonstrated that anti PD-1*1 IL7v*1 induced a robust and long-term specific memory anti-tumor response in the absence of any new treatment.

Although Anti PD-1*1 IL7v*1 has lower affinity for IL-7R, this construction demonstrated unexpected higher efficiency than the anti PD-1*IL-7wt*1 construction and conserves its antagonist anti PD-1 activity like anti PD-1 *2 antibody in a PD-1 sensitive tumor model. These data emphasize that the anti PD-1*1 IL7v*1 is a preferred construction to maintain blocking activity of PD-1 inhibitory receptor in vivo. Inventors suppose that the mutation of IL-7 will balance the affinity of the bifunctional antibody toward PD-1+ tumor specific T cells over PD-1− CD127+ non tumor specific T cells (as described in FIG. 11D) resulting in better efficacy of the drug in vivo.

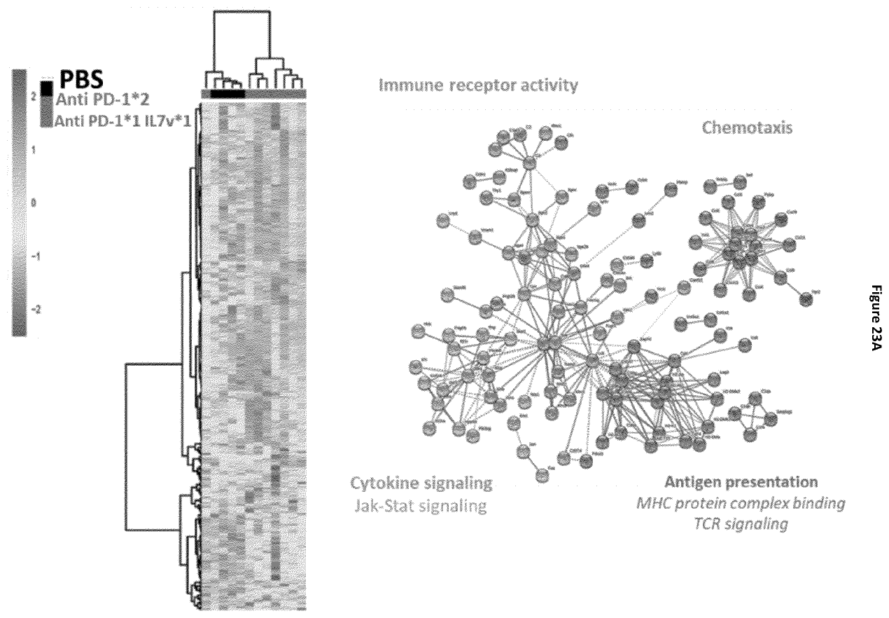

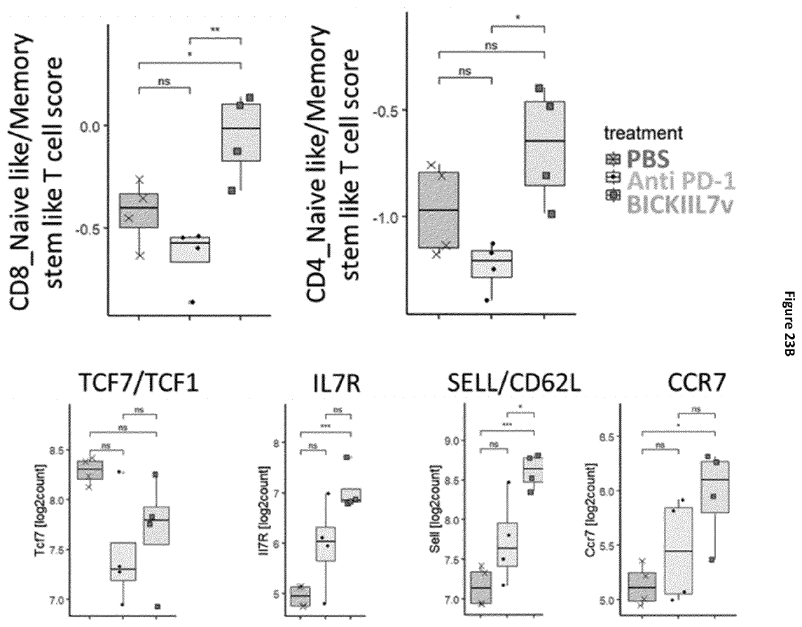

To evaluate efficacy in anti PD(L)1 refractory model to mimic primary resistance in cancer patients, a murine model of hepatocellular carcinoma Hepa1.6 was selected. It is an orthotopic syngeneic model implemented in immunocompetent mice (expressing Human PD-1). This model is of particular interest due to tumor T cell exclusion from the tumor described (Gauttier V et al. 2020, Clin Invest, 130, 6109-6123). Efficacy of Anti PD-1*1 IL7v*1 having low affinity for IL7R versus anti PD-1*1 IL7wt*1 having high affinity for IL7R was compared side by side in the same experiment. In separate groups, mice were treated with PBS (control), anti PD-1*2 or isotype*1 IL7*v*1 (homolog construction of the bispecific antibody targeting an anti-viral protein envelope and used as isotype control for the experiment) at same drug exposure concentration. Anti PD-1*1 IL7v*1 achieved complete tumoral responses at 60% clearly superior to the anti PD-1*1 IL7wt*1 constructed with high affinity wild type IL7 (47% of Complete Responses only) as shown on FIG. 22. In this model, anti PD1 antibody has, as expected, no efficacy. Moreover, Isotype*1 IL7v*1 has neither efficacy in this model demonstrating that combining anti PD-1 and IL-7 treatment using anti PD-1/IL7 construction is a good therapeutic strategy to enhance T cell activation and anti-tumor response in a PD-1 refractory model.

The memory response induced by anti PD-1*1 IL7v*1 treatment has also been tested in this model and the same absence of tumor has been observed.

Altogether these data confirm the superior efficacy of an anti PD-1/IL-7 construction with one anti PD-1 valency and one IL-7 cytokine mutated (W142H) having lower affinity for its CD127 receptor.

### Example 12: Anti PD-1*1 IL-7v*1 Demonstrated In Vivo Efficacy in Anti PD-1 Refractory Model is Correlated with Strong Transcriptional Activity of Anti PD-1 Receptor and an Intratumoral Proliferation of Stem Like Memory CD8 T Cell Subpopulation (TCF1+Tox− Cells)

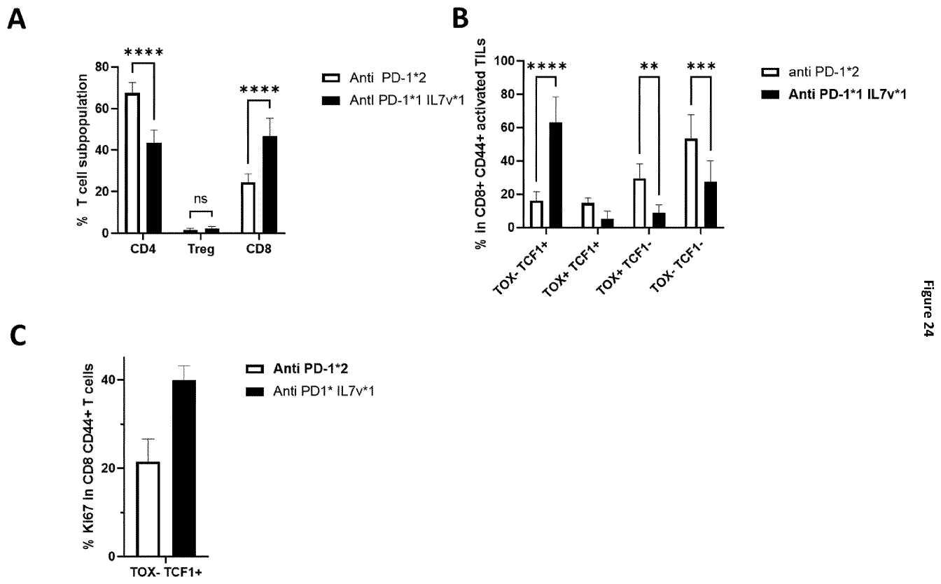

Transcriptomic analysis of the whole tumor was also performed to better understand the effect of anti PD-1*1 IL7v*1 into the tumor microenvironment. Gene expression was detected and quantified using Nanostring technology (nCounter® PanCancer Immune profiling panel). Data are normalized to multiple references genes included in panel with a background thresholding to the geometric mean of negatives controls. The differential expression of the genes (DEG) was analyzed using R package. Unsupervised hierarchical clustering heatmap of the DEG from the DESeq2 analysis of the FIG. 23 shows that transcriptional expression pattern is highly similar between Anti PD-1 and anti PD-1*1 IL7W14H*1 group and significantly different from PBS group, suggesting that the anti PD-1 domain of anti PD-1*1 IL7v*1 construction conserved its antagonist bioactivity in vivo despite its one anti PD-1 valency. A Protein-Protein Interaction Networks Functional Enrichment Analysis with STRING of the genes upregulated after treatment with anti PD-1*2 or anti PD-1*1 IL7W142H*1 compared to the PBS condition, identified several genes cluster involved in chemotaxis immune receptor activity, Jak-STAT cytokine signaling and antigen presentation (MHC protein complex binding and TCR signaling). Amongst gene that are differentially expressed between anti PD-1*2 and anti PD-1*1 IL7W142H*1, the inventors observed a significant upregulation of CD8 or CD4 T early activated/memory stem like T cell signature associated with expression of TCF7, CCR7, SELL, IL7R genes in anti PD-1*1 IL7W142H*1 group compared anti-PD-1 group and PBS group (FIG. 23B) using Single sample GSEA (ssGSEA) signature algorithm from the R package GSVA. On the opposite, an upregulation of exhausted CD8 T cell genes (LAG3, PRF1, CD8A, HAVRC2, GZMB, CD8B1, KLRD1, TNFRSF9, TIGIT, CTSW, CCL4, CD63, IFNG, CXCR6, FASL, CSF1) is observed in anti PD-1 treated group as expected. Gene signatures of Exhausted T cells and Naïve like/Stem like memory T cells signature was adapted from (Andreatta et al;, Nature comm 2021), which define different T cell subsets in cancer using Single cell transcriptomic analysis.

Phenotyping analysis by flow cytometry of the CD8 T cell infiltrating lymphocytes was also performed ex vivo to further characterized the population induced by anti PD-1*1 IL7v*1 treatment. Despite the T cell exclusion from the Tumor described initially in this resistant model, the tumor infiltrating lymphocytes (TILs) composition is strongly increased after Anti PD-1*1 IL7W142H*1 treatment and the product modified dramatically the T cell subsets (FIGS. 23 B and C, 24 C). Flow cytometry analysis demonstrated that anti PD-1*1 IL7 W142H*1 modify the composition of the tumor microenvironment, and favors accumulation of CD8 T cells over CD4 while sparing Treg (FIG. 24A). A high increase in the percentage of CD8+ CD44+ activated T cells with a phenotype of stem-like memory T cells (CD3+CD8+CD44+TCF1+TOX−) is observed following treatment (FIG. 24B) which also express ki67 proliferating marker (FIG. 24C). Anti PD-1 treatment induces accumulation of TOX−TCF1- or TOX+ TCF1-associated exhausted phenotype into the tumor (Utzschneider et al., Immunity 2016, 45, 415-427; Mann et al., 2019 Nature immunology, 20, 1092-1094). These data corroborate with transcriptomic analysis and further determine that the T cell activated by anti PD-1*1 IL7W142H*1 molecules express CD44 activation marker suggesting that this T cell subset is not a naïve T cell subset but rather an early activated steam like memory T cell subset (TCF1+TOX−). These data also confirm the example 9 describing the efficacy of anti PD-1*1 IL7W142H*1 to promote accumulation and proliferation of stem like memory T cells in vivo in another tumor model.

### Example 13: Anti PD-1*1 IL7v*1 Maintains Survival of Chronically Stimulated Human T Cells and Induce Proliferation of TCF1+ T Cells

To confirm effect of anti PD-1*1IL7v*1 on human T cells, inventors have tested the effect of anti PD-1/IL7 construction in chronic antigen stimulation model in vitro. Human PBMCs were repeatedly stimulated on CD3 CD28 coated plate (3 μg/mL of OKT3 and 3 μg/mL, CD28.2 antibody) every 3 days. At each stimulation, anti PD-1*1 IL7v*1 (Anti PD-1*1 IL7W142H*1) construction, isotype control or anti PD-1*1 antibody was added in the culture. Twenty-four hours following the fifth stimulation, T cell viability and phenotype were assessed by flow cytometry.

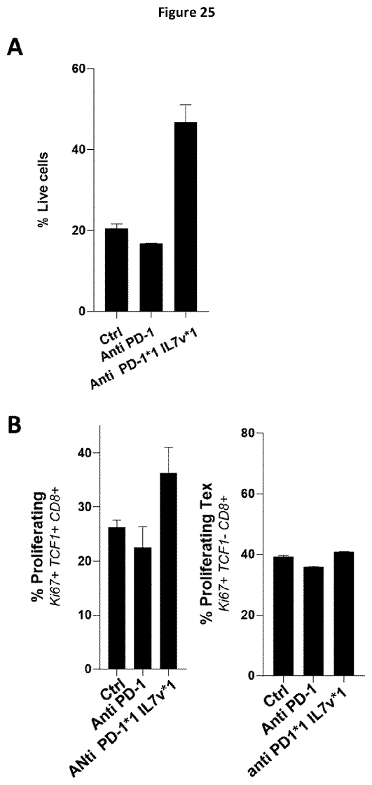

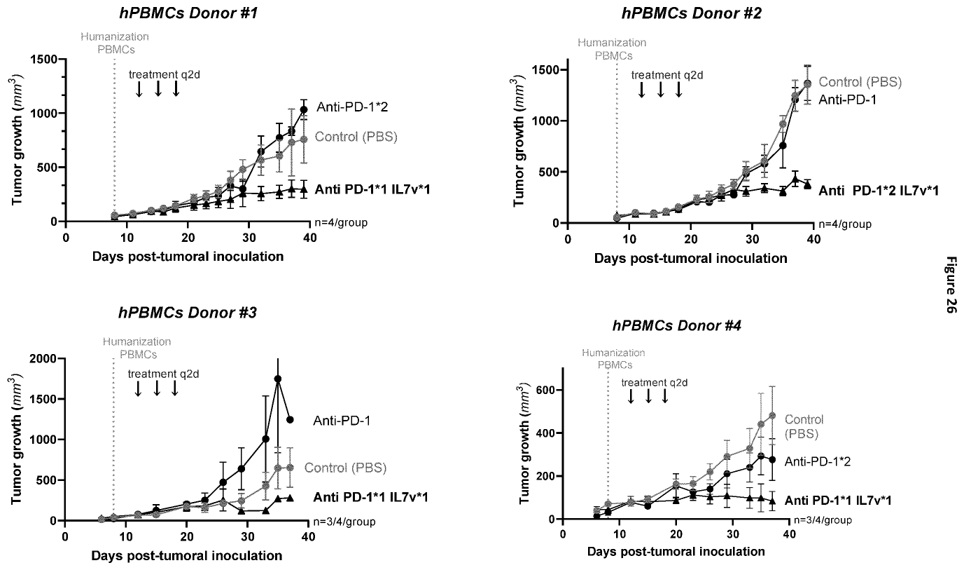

FIG. 25A shows that Anti PD-1*1 IL7W142H*1 maintains survival of chronically exhausted T cells compared to anti PD-1 treatment. Phenotypic analysis (FIG. 25B) of the T cells demonstrated that anti PD-1*1 IL7v*1 promotes specific proliferation and maintenance of TCF1+ CD8 T cell subset. TCF1+ T cell population is described as stem-like T cell population capable of self-renewal and long-term effective response. These results allow to anticipate long term effects in solid tumors by reinvigorating TILs with Anti PD-1*1 IL7v*1 in early stage of cancer (adjuvant or neo adjuvant situations) preventing exhausted T cell proliferation in primary or secondary resistance to Immune oncology treatment or other cancer treatment, and in various immune tumor escape situation.

### Example 14: Anti PD-1*1 IL-7v*1 Demonstrated In Vivo Monotherapy Efficacy in Different Humanized Model Resistant to PD-1 Therapy

In the Triple Negative Breast cancer (TNBC) model (immunodeficient mice subcutaneously implanted with Breast cancer cells MDA-MB231), mice were humanized with human Peripheral blood mononuclear cell (PBMC) from 4 different donors then treated with PBS, anti PD-1*2 or anti PD-1*1 IL7W142H*1 bifunctional antibody. In all PBMCs donor tested, anti PD-1*1 IL7v*1 reduced tumor growth whereas anti PD-1*2 has no effect alone (FIG. 26).

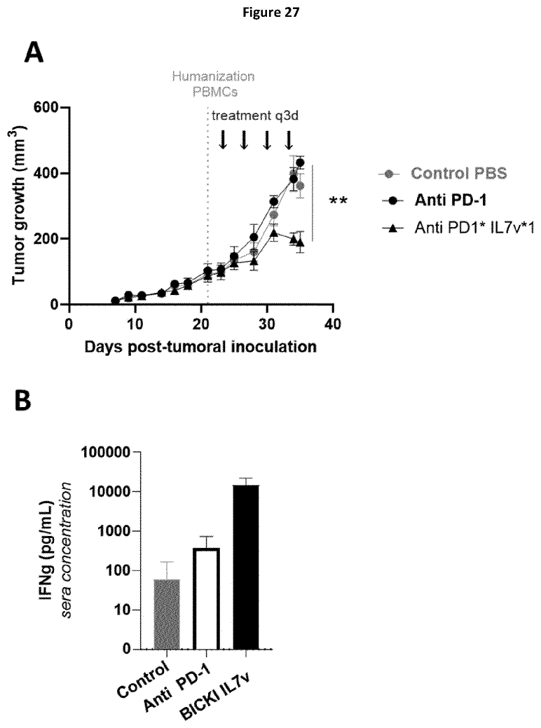

In another humanized mouse model, a Lung cancer model (A549), efficacy of anti PD-1*1 IL7v*1 was confirmed versus anti PD-1*1 associated with increased in IFNg secretion in the sera of that anti PD-1 *1 treated mice (Day 34) (FIG. 27). Both of this model demonstrate that anti PD-1*1IL7v can also modulate human immune-mediated antitumor response in vivo to higher extent than anti PD-1*2.

### Example 15: Anti PD-1*1 IL7v *1 Demonstrated Better Pharmacokinetic Profile Compared to Anti PD-1*1 IL7wt*1 Molecule in Cynomolgus Monkeys

Cynomolgus monkeys were intravenously injected with one dose of anti PD-1*1 IL7wt*1 (0.8 mg/kg, 4.01 mg/kg) or one dose of anti PD-1*1 IL7v*1 (Anti PD-1*1 IL7 W142H*1) 0.8 mg/kg, 4.01 mg/kg or 25 mg/kg). After injection, sera were collected at multiple time points to quantify anti PD-1 IL7 constructions by ELISA immunoassay using MSD technology. Briefly, human PD1 protein was immobilized and sera anti PD-1*1 IL7*1 antibody was added. ELISA were revealed with a sulfo-tagged anti-human kappa light chain monoclonal antibody.

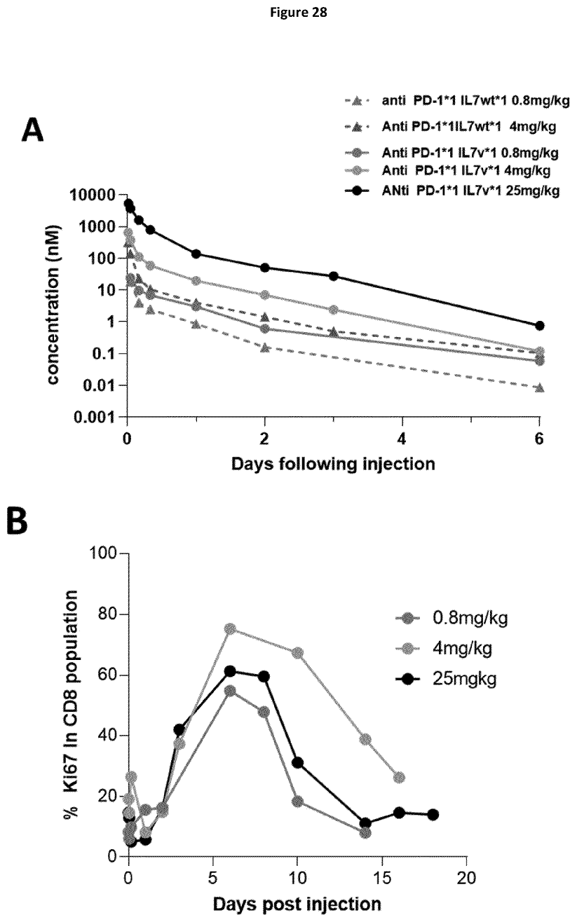

Pharmacokinetic data were linear, and dose related for both constructions. However, with anti PD-1*1 IL7v*1 construction, a better pharmacokinetic profile is observed compared to the anti PD-1*1 IL7wt*1 construction (FIG. 28) (Area under the curve 29.6 vs 108, IL7wt vs IL7v at the dose 4.01 mg/kg). Interestingly, Anti PD-1*1 IL7v*1 with low affinity to the IL7 receptor induced proliferation of CD8 T cells in vivo until day 10-14, showing that the biological effect of the drug is extended beyond pharmacokinetics exposure. These data allow a new pharmacodynamic model measuring a long-term effect on T cell subsets in Non-Human Primate and applicable to human situation: namely CD8+ T cell proliferation after only one injection of anti PD-1*1 IL7v*1 bifunctional antibody.

### Example 16: Anti PD-1*1 IL7v *1 Constructed with an IgG1 N297A Isotype or with a LALA PG IgG1 Isotype Has the Same Potency to Activate pSTAT5 Signaling into Human T Cells

In the examples 1 to 15, the format IgG1 N297A was used for the Anti PD-1*1/cytokines construction. Inventors have tested another Fc silent format with LALA PG additional mutation, these mutations were described to fully abrogate ADCC, ADCP and CDC activity since the LALA PG mutation impairs binding to FcR receptors.

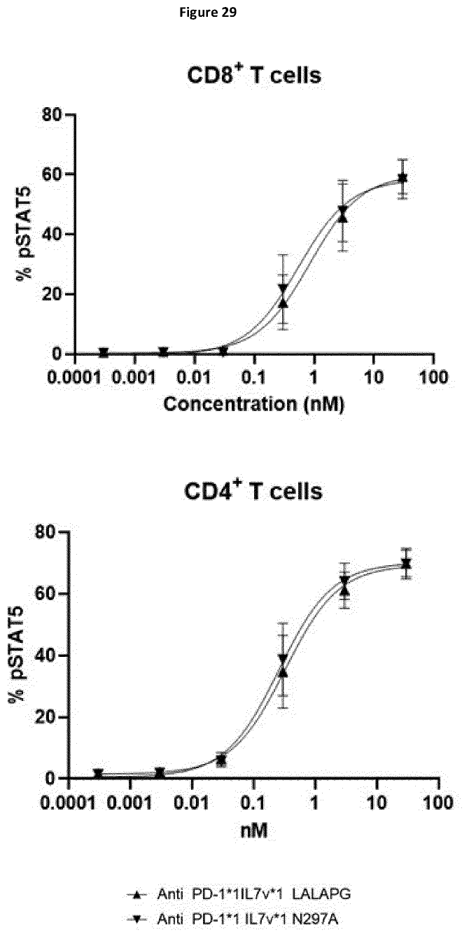

IL-7R Activity of the Anti PD-1*1 IL7W142H*1 as assessed by pSTAT5 activity (FIG. 29). No difference in term of activity on CD4 and CD8 human T cells was noticed between the 2 constructions indicating that the present invention can be constructed with different Fc silent isotypes.

### Example 17. Anti PD-1*1/protX*1 Demonstrated Absence of In Vivo Toxicity

In vivo toxicity was assessed after the intraperitoneally injection of either three doses at 5 or 20 mg/kg, or single high dose of 50 mg/kg or 100 mg/kg of bifunctional molecules into C57BL/6 mice. Weight was controlled twice before treatment (one and four days before), on the day of treatment and every day following treatment. PBS was used as control.

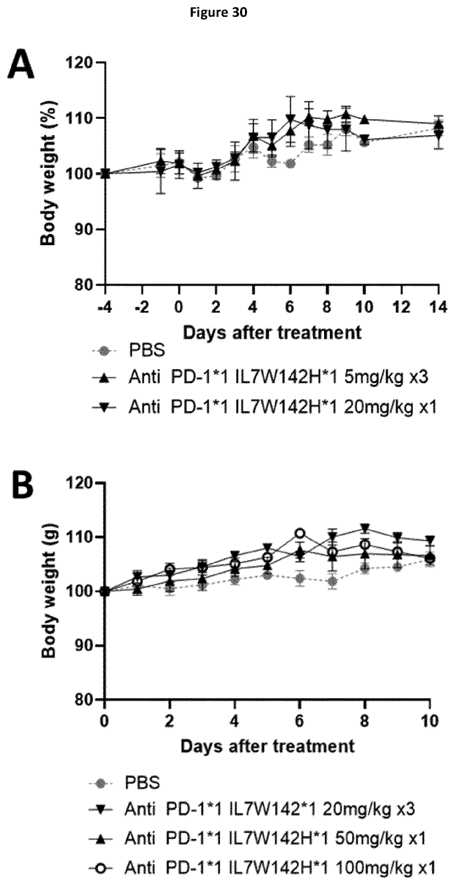

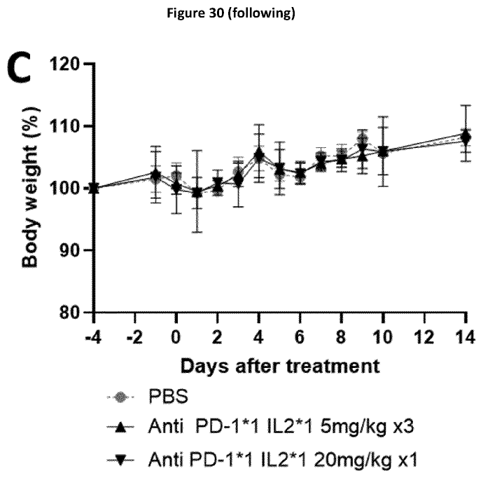

Results: FIG. 30 shows that mice treated with Anti PD-1*1/protX*1 manifested a similar weight than PBS control injected mice, suggesting the injection of Anti PD-1*1/protx*1 (IL7 or IL2), either chronically or acutely, does not induce toxicity even at high dose. Additionally, mice did not exhibit outer sign of distress.

### Material and Methods

### ELISA Binding PD1

For activity ELISA assay, recombinant hPD1 (Sino Biologicals, Beijing, China; reference 10377-H08H) was immobilized on plastic at 0.5 μg/ml in carbonate buffer (pH9.2) and purified antibody were added to measure binding. After incubation and washing, peroxidase-labeled donkey anti-human IgG (Jackson Immunoresearch; USA; reference 709-035-149) was added and revealed by conventional methods.

### ELISA Antagonist: Competition between PDL1 and Humanized Anti-PD1

Competitive ELISA assay was performed by PD-1:PD-L1 Inhibitor Screening ELISA Assay Pair (Acro Biosystems; USA; reference EP-101). In this assay, recombinant hPDL1 was immobilized on plastic at 2 μg/ml in PBS pH7.4 buffer. Purified antibody (at different concentrations) were mixed with 0.66 μg/ml final (fix concentration) of biotinylated Human PD1 (AcroBiosystems; USA; reference EP-101) to measure competitive binding for 2 h at 37° C. After incubation and washing, peroxidase-labeled streptavidin (Vector laboratoring; USA; reference SA-5004) was added to detect Biotin-PD-1Fc binding and revealed by conventional methods.

### pSTAT5 Analysis

PBMCs isolated from peripheral blood of human healthy volunteers were incubated 15 minutes with anti PD-1/IL-7 molecule at 37° C.

To determine cis activity, U937 transduced with CD127 and PD-1 were mixed with U937 transduced with CD127+ only. Cells were stained with cell proliferation dye (CPDe450 or CPDe670, thermofisher) mixed at a ratio 1:1 and treated with the tested molecule during 15 minutes at 37° C. Each cell subset was labeled with Cell proliferation dye (CPDe450 or CPDe670) prior to coculture. Cells were then fixed, permeabilized and stained with an AF647 labeled anti-pSTAT5 (clone 47/Stat5(pY694), BD Bioscience). For human PBMCs, pSTAT5 activation was evaluated into CD3+ T cell population. For the U937 assay, pSTAT5 activation was evaluated into U937 PD1+ CD127+ cells and U937 CD127+ cells.

### Cellular Binding Analysis

U937 transduced with CD127 and PD-1 were mixed with U937 transduced with CD127+ only. Cells were stained with cell proliferation dye (CPDe450 or CPDe670, thermofisher) mixed at a ratio 1:1. Cells were stained with Yellow/live dead fixable staining (Thermofisher), then stained with human Fc Block diluted in PBS 2% human serum (BD Bioscience). Cells were then stained with serial concentrations of the tested molecules and revelation of the antibodies was performed with an anti-human IgG-PE antibody (Biolegend, clone HP6017) and analyzed by flow cytometry

### Pharmacokinetics of the Anti PD-1/protX In Vivo

To analyze the pharmacokinetics, a single dose of the molecule was intra-orbitally or intraperitoneally or intravenously (retroorbital) injected into C57bl6JrJ mice (female 6-9 weeks) Drug concentration in the plasma was determined by ELISA using an immobilized anti-human light chain antibody (clone NaM76-5F3) diluted serum containing IgG fused 1167. Detection was performed with a peroxidase-labeled donkey anti-human IgG (Jackson Immunoresearch; USA; reference 709-035-149) and revealed by conventional methods.

### T Cell Activation Assay Using Promega Cell-Based Bioassay

The capacity of anti-PD-1 antibodies restore T cell activation was tested using Promega PD-1/PD-L1 kit (Reference J1250). Two cell lines are used (1) Effector T cells (Jurkat stably expressing PD-1, NFAT-induced luciferase) and (2) activating target cells (CHO K1 cells stably expressing PDL1 and surface protein designed to stimulate cognate TCRs in an antigen-independent manner. When cells are cocultured, PD-L1 /PD-1 interaction inhibits TCR mediated activation thereby blocking NFAT activation and luciferase activity. The addition of an anti- PD-1 antibody blocks the PD-1 mediated inhibitory signal leading to NFAT activation and luciferase synthesis and emission of bioluminescence signal. Experiment was performed as per as manufacturer recommendations. Serial dilutions of the tested molecules were tested. Four hours following coculture of PD-L1+ target cells, PD-1 effector cells and tested molecules, BioGlo™ luciferin substrate was added to the wells and plates were read using Tecan™ luminometer.

### In Vivo Proliferation

A single dose of bifunctional molecules (34 nM/kg) was intraperitoneally injected to C57bl6JrJ mice (female 6-9 weeks) bearing a subcutaneous MC38 tumor. Mice were treated with one dose (34 nM/kg) via intraperitoneal injection. On Day 4 following treatment, Blood was collected, and T cells were stained with an anti CD45, CD3, anti CD8, anti CD4 antibody and an anti ki67 antibody to quantify proliferation by flow cytometry.

### In Vivo Humanized PD1 Knock In Mouse Model

Efficacy of the anti PD-1/IL-7 molecules was assessed in vivo in syngeneic immunocompetent mouse model genetically modified to express human PD-1 (exon 2). For the orthotopic mesothelioma model, AK7 mesothelial cells were intraperitoneally injected (3e6 cell/mouse) then treated at Day 4/6/8 at equivalent drug exposure dose [anti PD-1*2 (1 mg/kg), anti PD-1*1/IL-7*1 at 4 mg/kg]. Injected AK7 cells stably express luciferase allowing generation of in vivo bioluminescence signal following intraperitoneal injection of D-luciferin (3 μg/mouse, GoldBio, Saint Louis MO, USA, Reference 115144-35-9). Ten minutes following luciferin injection, bioluminescence signal was measured by Biospace Imager on the dorsal side and ventral side of the mouse for 1 minute. Data were analyzed in photon per second per cm2 per steradian and represent the mean of the dorsal and ventral signal. Each group represents mean +/− SEM of 5 to 7 mice per group. For the Hepatocarcinoma model, Hepa1.6 hepatocarcinoma cells were subcutaneously injected with2.5e6 cells in the portal vein. Mice were treated then treated at Day 4/6/8 at equivalent drug exposure dose [anti PD-1*2 (1 mg/kg), anti PD-1*1/IL-7*1 at 4 mg/kg].

### Antibodies and Bifunctional Molecules

The following antibodies and bifunctional molecules have been used in the different experiments disclosed herein.

| | SEQ ID NO | SEQ ID NO | SEQ ID NO |
|---|---|---|---|
| Molecule | (if relevant, Chain A) | (if relevant, Chain B) | Light Chain |
| antiPD1*2 | 35 | — | 80 |
| antiPD1*1 | 75 | 90 | 80 |
| antiPD-1*2 IL-7 W142H*2 or | 92 | — | 80 |
| anti PD-1*2/IL-7v*2 | | | |
| antiPD-1*2 IL-7 W142H*1 or | 81 | 83 | 80 |
| anti PD-1*2/IL-7v*1 | | | |
| antiPD-1*1 IL-7 W142H*1 or | 75 | 83 | 80 |
| anti PD-1*1/IL-7v*1 | | | |
| antiPD-1*1 IL-7 W142H*2 or | 76 | 83 | 80 |
| anti PD-1*1/IL-7v*2 | | | |
| antiPD-1*2 IL-7 WT*2 | 91 | — | 80 |
| antiPD-1*2 IL-7 WT*1 | 81 | 93 | 80 |
| antiPD-1*1 IL-7 WT*1 | 75 | 93 | 80 |
| antiPD-1*2 IL-2*1 | 81 | 84 | 80 |
| antiPD-1*1 IL-2*1 | 75 | 84 | 80 |
| antiPD-1*2 IL-15*1 | 81 | 85 | 80 |
| antiPD-1*1 IL-15*1 | 75 | 85 | 80 |
| antiPD-1*2 IL-21*1 | 81 | 86 | 80 |
| antiPD-1*1 IL-21*1 | 75 | 86 | 80 |

1-16. (canceled)

17. A bifunctional molecule comprising a single antigen binding domain that binds to a target specifically expressed on immune cells surface and a single immuno-stimulating cytokine,
wherein the molecule comprises a first monomer comprising an antigen-binding domain covalently linked via C-terminal end to N-terminal end of a first Fc chain, optionally via a peptide linker, and a second monomer comprising a complementary second Fc chain devoid of antigen-binding domain and of the immuno-stimulating cytokine;
wherein either i) the immuno-stimulating cytokine is covalently linked to the C-terminal end of said first Fc chain, optionally via a peptide linker; or ii) the single antigen binding domain comprises a heavy variable chain and a light variable chain and the immuno-stimulating cytokine is covalently linked to the C-terminal end of the light chain;
wherein the target specifically expressed on immune cells surface is selected from the group consisting of PD-1, CD28, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CD3, PDL2, and PDL1; and
wherein the immuno-stimulating cytokine is selected from the group consisting of IL-2 (IL being interleukin), IL-4, IL-5, IL-6, IL-12A, IL-12B, IL-13; IL-15, IL-18, IL-21, IL-23, IL-24; IFNα, IFNβ, BAFF, LTα, and LTβ, or a variant thereof having at least 80% of identity with the wildtype protein.

18. The bifunctional molecule of claim 17, wherein the immuno-stimulating cytokine is linked at the C-terminal end of first Fc chain.

19. The bifunctional molecule of claim 17, wherein the first Fc chain and the second Fc chain form a heterodimeric Fc domain.

20. The bifunctional molecule of claim 17, wherein the immuno-stimulating cytokine is selected from the group consisting of IL-2, IL-15, and IL-21, or a variant thereof having at least 80% of identity with the wildtype protein.

21. The bifunctional molecule of claim 17, wherein the immuno-stimulating cytokine is IL-2 or a variant thereof having at least 90% of identity with SEQ ID NO: 87, or is an IL-2 variant comprising one of the following substitution combinations relative to SEQ ID NO: 87: R38E and F42A; R38D and F42A; F42A and E62Q; R38A and F42K; R38E, F42A, and N88S; R38E, F42A, and N88A; R38E, F42A, and V91E; R38E, F42A, and D84H; H16D, R38E and F42A; H16E, R38E and F42A; R38E, F42A and Q126S; R38D, F42A and N88S; R38D, F42A and N88A; R38D, F42A and V91E; R38D, F42A, and D84H; H16D, R38D and F42A; H16E, R38D and F42A; R38D, F42A and Q126S; R38A, F42K, and N88S; R38A, F42K, and N88A; R38A, F42K, and V91E; R38A, F42K, and D84H; H16D, R38A, and F42K; H16E, R38A, and F42K; R38A, F42K, and Q126S; F42A, E62Q, and N88S; F42A, E62Q, and N88A; F42A, E62Q, and V91E; F42A, E62Q, and D84H; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, V91E, and C125A; R38E, F42A, D84H, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, V91E, and C125A; R38D, F42A, D84H, and C125A; H16D, R38D, F42A, and C125A; H16E, R38D, F42A, and C125A; R38D, F42A, C125A, and Q126S; R38A, F42K, N88S, and C125A; R38A, F42K, N88A, and C125A; R38A, F42K, V91E, and C125A; R38A, F42K, D84H, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, V91E, and C125A; F42A, E62Q, and D84H, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, V91E, and C125A; F42A, D84H, and C125A; H16D, F42A, and C125A; H16E, F42A, and C125A; F42A, C125A and Q126S; F42A, Y45A and L72G; and T3A, F42A, Y45A, L72G and C125A.

22. The bifunctional molecule of claim 17, wherein the immuno-stimulating cytokine is IL-15 or a variant thereof having at least 90% of identity with SEQ ID NO: 88, or an IL-15 variant comprising one of the following substitutions relative to SEQ ID NO: 88: N1D, V3I, V3M, V3R, N4D, D8N, D8A, K11L, K11M, K11R, D30N, D61N, E64Q, N65D, N71D, N71S, N72D, N72A, N72R, N72Y, S73I, N77A, N79D, N79E, N79S, Q108E, N112D, N112H, N112M, N112Y, N4D/N65D, D30N/N65D, D30N/E64Q, D30N/E64Q/N65D, N1D/D61N, N1D/E64Q, N4D/E64Q, D8N/D61N, D8N/E64Q, D61N/E64Q, N1D/D30N, E64Q/Q108E, N1D/N4D/D8N, D61N/E64Q/N65Q, N1D/D61N/E64Q/Q108E, N4D/D61N, N4D/D61N/E64Q/Q108E, N1D/N65D, N1D/Q108E, N4D/D30N, D30N/Q108E, N65D/Q108E, D30N/Q180E, E64Q/N65D, D61N/E64Q/N65D, N1D/N4D/N65D, N71S/N72A/N77A, and N4D/D61N/N65D.

23. The bifunctional molecule of claim 17, wherein the immuno-stimulating cytokine is IL-21 or a variant thereof having at least 90% of identity with SEQ ID NO: 89 or an IL-21 variant comprising one of the following substitutions relative to SEQ ID NO: 89: R5A, R5D, R5E, R5G, R5H, R5I, R5K, R5L, R5M, R5N, R5Q, R5S, R5T, R5V, R5Y, I8A, I8D, I8E, I8G, I8N, I8S, R9A, R9D, R9E, R9G, R9H, R9I, R9K, R9L, R9M, R9N, R9Q, R9S, R9T, R9V, R9Y, R11D, R11S, Q12A, Q12D, Q12E, Q12N, Q12S, Q12T, Q12V, L13D, I14A, I14D, I14S, D15A, D15E, D15I, D15M, D15N, D15Q, D15S, D15T, D15V, I16D, I16E, Q19D, Y23D, R65D, R65G, R65P, I66D, I66G, I66P, N68Q, V69D, V69G, V69P, S70E, S70G, S70P, S70Y, S70T, K72D, K72G, K72P, K72A, K73A, K73D, K73E, K73G, K73H, K73I, K73N, K73P, K73Q, K73S, K73V, K75D, K75G, K75P, R76A, R76D, R76E, R76G, R76H, R76I, R76K, R76L, R76M, R76N, R76P, R76Q, R76S, R76T, R76V, R76Y, K77D, K77G, K77P, P78D, P79D, S80G, S80P, E109K, R110D, K112D, S113K, Q116A, Q116D, Q116E, Q116I, Q116K, Q116L, Q116M, Q116N, Q116S, Q116T, Q116V, K117D, I119A, I119D, I119E, I119M, I119N, I119Q, I119S, I119T, H120D and L123D, R5E and R76E, R5E and R76A, R5A and R76A, R5Q and R76A, R5A and R76E, R5Q and R76E, R9E and R76E, R9A and R76E, R9E and R76A, R9A and R76A, D15N and S70T, D15N and I71L, D15N and K72A, D15N and K73A, S70T and K73Q, S70T and R76A, S70T and R76D, S70T and R76E, I71L and K73Q, I71L and R76A, I71L and R76D, I71L and R76E, K72A and K73Q, K72A and R76A, K72A and R76D, K72A and R76E, K73A and R76A, K73A and R76D, or K73A and R76E.

24. The bifunctional molecule of claim 17, wherein the antigen-binding domain is a Fab domain, a Fab′, a single-chain variable fragment (scFV) or a single domain antibody (sdAb).

25. The bifunctional molecule of claim 17, wherein the target specifically expressed on immune cells surface is selected from the group consisting of PD-1, CTLA-4, BTLA, TIGIT, LAG3 and TIM3.

26. The bifunctional molecule of claim 17, wherein the antigen binding domain binds to PD-1.

27. The bifunctional molecule of claim 17, wherein the antigen binding domain comprises: (i) a heavy chain comprising a CDR1 of SEQ ID NO: 51, a CDR2 of SEQ ID NO: 53 and a CDR3 of SEQ ID NO: 55, 56, 57, 58, 59, 60, 61 or 62; and (ii) a light chain comprising a CDR1 of SEQ ID NO: 64 or 65, a CDR2 of SEQ ID NO: 66 and a CDR3 of SEQ ID NO: 16.

28. An isolated nucleic acid sequence or a group of isolated nucleic acid molecules encoding the bifunctional molecule according to claim 17.

29. A host cell comprising the isolated nucleic acid according to claim 28.

30. A pharmaceutical composition comprising the bifunctional molecule according to claim 17 and a pharmaceutically acceptable carrier.

31. A method of treating cancer or an infectious disease comprising administering a bifunctional molecule according to claim 17 to a subject having cancer or an infectious disease.

32. The method according to claim 31, said method stimulating effector memory stem like T cells.
